(19) United States
(12) Patent Application Publication
(10) Pub. No.: US 2011/0159500 A1
(43) Pub. Date: Jun. 30, 2011

(54) COMPOSITIONS INCLUDING GINGER FOR THE AMELIORATION OR PREVENTION OF INFLAMMATORY CONDITIONS

(75) Inventors: Christina Khoo, Duxbury, MA (US); Inke Paetau-Robinson, Auburn, KS (US); Nolan Zebulon Frantz, Meadville, PA (US)

(73) Assignee: Hill's Pet Nutrition, Inc., Topeka, KS (US)

(21) Appl. No.: 12/977,718

(22) Filed: Dec. 23, 2010

Related U.S. Application Data
(60) Provisional application No. 61/290,788, filed on Dec. 29, 2009.

(51) Int. Cl. C12Q 1/68; G01N 33/53; C12Q 1/37; C12Q 1/26; C40B 30/04; G01N 33/559; G01N 27/447

(52) U.S. Cl. 435/6.12; 435/7.4; 435/7.92; 435/7.94; 435/23; 435/25; 506/9; 204/461; 204/450

(57) ABSTRACT

The invention encompasses a method for diagnosing an arthritic condition and a gastrointestinal inflammatory disorder in a companion animal. The invention also encompasses a method for identifying ingredients for a pet food composition that are suitable for preventing, ameliorating the symptoms of, or treating an arthritic condition or gastrointestinal inflammatory disorder.

This application claims priority of U.S. Provisional Patent Application No. 61/290,788 filed on Dec. 29, 2009, which is incorporated herein by reference.

### BACKGROUND OF THE INVENTION

Ginger has long been a culinary ingredient, enjoyed particularly for the flavor it provides. Native to southern Asia, ginger is a 2- to 4-foot perennial that produces grass-like leaves up to a foot long and almost an inch wide. Ginger root, as it is called in the grocery store, consists of the underground stem of the plant, with its bark-like outer covering scraped off. Ginger has long been used as a health supplement for humans.

Ginger's modern use dates back to the early 1880s, when it was noticed that ginger-filled capsules reduced nausea during flu. Ginger was then approved as a treatment for indigestion and motion sickness. Ginger has since become widely accepted as a treatment for human nausea. Even some conventional medical texts suggest ginger for the treatment of nausea and vomiting of pregnancy, although others are more cautious.

Accordingly, the inventors have developed pet food compositions including ginger, which are useful in treating inflammatory conditions, including gastrointestinal disorders and arthritis, and for improvement of the well-being of companion animals, for example, cats and dogs.

It is generally accepted in the scientific community that genes play a role in animal development and that the regulation of gene expression plays a key role in the development of some diseases or conditions that affect an animal's health and well being. Similarly, the differential expression of genes is one factor in the development of such diseases and conditions and the evaluation of gene expression patterns has become widely recognized as crucial to understanding the development and control of such diseases and conditions at the molecular level. To advance the understanding of genes and their relationship to disease, a number of methods have been developed for studying differential gene expression, e.g., DNA microarrays, expressed tag sequencing (EST), serial analysis of gene expression (SAGE), subtractive hybridization, subtractive cloning and differential display (DD) for mRNA, RNA-arbitrarily primed PCR (RAP-PCR), Representational Difference Analysis (RDA), two-dimensional gel electrophoresis, mass spectrometry, and protein microarray based antibody-binding for proteins.

Arthritis is a musculoskeletal disorder. Osteoarthritis is the most common type of arthritis in animals and humans. Osteoarthritis is a degenerative joint disease commonly occurring in humans and companion animals and the disease is characterized by degenerative changes in the articular cartilage, with loss of proteoglycan and collagen and proliferation of new bone formation at articular margins. These changes are accompanied by a variable inflammatory response within the synovial membrane. A principal defect in hyaline cartilage at the articular surface of a joint is the alteration in the ratio of glycosaminoglycans to the collagen fiber content of the matrix. Bones directly underlying cartilage in the joints are called subchondral bones. These subchondral bones nourish the overlying cartilage which itself is devoid of blood vessels, nerves or lymphatic tissue.

A natural erosion of cartilage occurs with age, but may also result from excessive loads placed on joints, obesity, heredity, trauma, decreased circulation, poor bone alignment and repetitive stress may exacerbate the condition of the joint. It is postulated that free radical damage which may contribute to the development of osteoarthritis.

Cells of hyaline cartilage known as chondrocytes produce and maintain the surrounding extracellular matrix. Maintenance of homeostatis of the cartilage matrix depends upon catabolism of matrix proteins such as type II collagen and aggrecan. These proteins are digested and replaced by new proteins synthesized by chondrocytes. Catabolism is in part carried out by proteolytic enzymes such as matrix metalloproteinase (MMP) and aggrecanase proteins. In a normal animal, a balance is achieved between synthesis and degradation, thereby maintaining healthy cartilage. When the balance shifts to degradation, pathogenesis ensues and may result in joint inflammation and osteoarthritis.

A homeostatic condition in cartilage is dependent upon regulation through intercellular signaling between chondrocytes. Chondrocytes thus produce and respond to signaling molecules. Such signaling molecules may comprise cytokines and growth factors which may directly influence cellular metabolism. Intercellular signaling is complex and has not been fully characterized. Growth factor molecules such as TGF-beta are involved and believed to promote type II collagen production and to inhibit collagen cleavage. Cytokines, such as TNF-alpha and IL-1-beta, also play a role. These cytokines are believed to promote production of proteases that may degrade cartilage. Numerous other complex interactions are believed to be occurring as a result of intercellular signaling.

Due to the complexity of the intercellular signaling process, it is highly desirable to understand at a genetic level the interactions that are taking place. Detection of dysregulated genes in a pre-arthritic or an arthritic condition is helpful in understanding the biology of abnormal musculoskeletal joint disorders, especially on a genome-wide basis. A more detailed understanding of the biological pathways involved through gene expression profiling will aid in the development of salutary pharmaceutical, nutraceutical and nutritional (dietary) interventions in the disease pathways. These approaches may enable prevention, early detection and treatment of the underlying abnormal musculoskeletal joint conditions as well as in monitoring the prognosis of such abnormal musculoskeletal joint disorders, especially in osteoarthritis. Dysregulated genes involved in the pathology of such disorders may serve as important biomarker to optimize selection of appropriate pharmaceutical, nutraceutical and nutritional (dietary) intervention.

There is yet to be identified a drug that reverses the course of osteoarthritis. Currently available therapeutic agents are employed to reduce inflammation and/or to relieve pain. Current therapy employs a class of drugs known as non-steroidal anti-inflammatory drugs (NSAIDs) to treat musculoskeletal joint disorders such as osteoarthritis, but these therapies have a variety of drawbacks, including, in particular, gastrointestinal disorders, and may also inhibit cartilage formation.

Large dogs may develop arthritis as they age. Large dog breeds are more susceptible to arthritis due to their increased mass and/or genetic disposition. Large dogs are not the only animals at risk of arthritis and other cartilage conditions. Arthritis and other degenerative joint diseases have been commonly recognized in dogs and such conditions have been shown to be prevalent in cats. Feline osteoarthritis is a disease primarily affecting aged felines ten years of age or older. Animals at risk of developing cartilage-affecting abnormal musculoskeletal joint disorders include, but are not limited to, mammals such as canine, feline, equine, hircine, ovine, porcine, bovine, human and non-human primate species, and birds including turkeys and chickens.

Irritable bowel disorders (“IBD”) are also characterized by inflammatory infiltrates within the lamina propria of the gastrointestinal tract. IBD encompasses segmental granulomatous enterocolitis, lymphoplasmacytic enteritis, eoisinophilic gastroenterocolitis, lymphocytic gastroenterocolitis, suppurative enterocolitis and histiocytic colitis. These specific types of irritable bowel disorders are characterized based on the type of inflammatory infiltrate found in the lamina propria. The inflammatory infiltrates can be quite variable in terms of severity and cell types, with lymphocytes and plasma cells being the most common cell types. Inflammatory infiltrates may involve the stomach, small bowel and colon. In the cat, for example, the stomach and small bowel are affected most often. In many cases, multiple segments of the bowel are involved and clinical signs may be mixed, reflecting the broad distribution of mucosal lesions. The severity of irritable bowel disorders varies from mild clinical signs to life-threatening protein-losing enteropathies.

Irritable bowel disorders may be marked by any of the following symptoms: abdominal pain, vomiting, diarrhea, hematochezia (bright red blood in stools), weight loss, and various associated complaints or diseases like arthritis, pyoderma gangrenosum, and primary sclerosing cholangitis. Irritable bowel disorders may also be a result of the following conditions: Crohn's disease, ulcerative colitis, overactive immune system, collagenous colitis, lymphocytic colitis, ischemic colitis, diversion colitis, Behcet's syndrome, infective colitis, and indeterminate colitis.

Mucosal inflammatory infiltrates are responsible for the clinical manifestations of IBD. Mucosal inflammation disrupts normal absorptive processes. Such disruption results in malabsorption and osmotic diarrhea. Altered gut permeability can result in leakage of fluid, protein and blood into the gut lumen. Malabsorbed fats, carbohydrates, and bile acids result in secretory diarrhea. Inflammatory mediators may also directly trigger intestinal secretion and mucus production by goblet cells. Mucosal inflammatory infiltrates may alter intestinal and colonic motility patterns, a mechanism attributed to the influence of prostaglandins and leukotrienes on smooth muscle. Inflammation of the stomach and small bowel stimulates receptors that trigger vomiting. In cats, for example, the most common clinical signs of irritable bowel disorders are chronic vomiting, diarrhea and weight loss.

Diet plays an important role in disease causation and progression because it is fundamentally involved in metabolism. Disease regulated genes are at some level regulated by nutritional factors. Thus, dietary components present in foods as nutrients may regulate gene expression at the transcriptional and translational level, as well as in certain post-translational modifications. They may similarly be involved in degradation and enzymatic activities. Nutrient levels may influence the equilibrium of metabolic pathways. Metabolic pathways are frequently complex and may involve many redundancies and interrelationships among different metabolic pathways. Altering the concentration of a single enzyme, growth factor, cytokine or metabolite may impact a number of metabolic pathways involved in disease-related physiology. Hormones and other cell signaling molecules are well-understood to be regulated by diet and are also known to be implicated in the development and progression of disease.

The same disease phenotype may result from disturbances in different metabolic pathways, and the genetic make-up of each animal differs, thereby causing variation in responses to the same factors, including nutritional and environmental factors. The interplay of genetic, nutritional and environmental factors is important in understanding the etiology, prevention, treatment and progression of diseases in animals. Finding gene expression responses to nutrients associated with various diseases and disorders permits formulation of diets for animals susceptible to disease such as abnormal arthritic disorders and abnormal inflammatory gastrointestinal disorders, and further permits diagnosis, treatment and monitoring the prognosis of the underlying disease or disorder.

Nutritional components influence gene expression, including mRNA production (transcription), mRNA processing, protein production (translation) and post-translational modifications, thereby influencing the overall metabolic status of an animal. As a result, the use of biomarker for early detection and monitoring of disease progression and/or genotype-based diets may enable prevention or treatment of diseases as well as new therapies to be developed for animals, particularly for companion animals. Diet is arguably the most important environmental factor affecting the phenotype of an animal, including susceptibility to disease.

Gene expression may be regulated through unstable processes that are controlled by activators and repressors of gene function. Nutritional status may cause significant changes in gene transcription rates. Macronutrients such as glucose, fatty acids and amino acids and micronutrients such as iron, zinc and vitamins can regulate gene expression. Various bioactive food components such as carotenoids, flavonoids, monoterpenes and phenolic acids may act as transcription factors affecting gene expression. These substances tend to have direct effects on gene expression. In other situations, substances like dietary fiber, which is fermented in the gut by bacteria, may lead to the production of nutrients such as short chain fatty acids. Such substances may act as indirect activators or repressors of gene expression.

Identification of nutrient-related changes upon transcription and translation may be detected in experiments of the type described in this specification. In view of the extensive array of genes profiled in the examples of this specification, alterations in gene expression and quantification are readily detected by the methods taught in this specification. Thus, dietary and metabolic gene expression signatures may be readily ascertained using the techniques taught in the Examples of this specification. Biomarkers of the invention are proteins and/or nucleic acids that are differentially expressed in animals. Biomarker expression can be assessed at the protein or nucleic acid level using various methods known to the skilled artisan.

Only very limited work has been done to date in screening the canine and feline genomes for gene expression profiles in response to nutritional components in the diet of these companion animals.

Studies in healthy populations of animals versus populations having a disease such as the abnormal arthritic and gastrointestinal inflammatory disorders described in this specification have not been extensively conducted. Little data is available with respect to the canine genome and far less with respect to the feline genome. Gene expression data contained in this specification identifies genes associated with cartilage degradation in dogs and cats. Such gene expression data enables identification of nutritional compositions capable of modulating expression of such genes in a favorable manner. This is also the case with respect to genes generally associated with inflammation. Analogous data in felines is additionally set forth in the specification, figures and examples of this specification.

Gene expression data contained in the specification and examples enables a variety of desirable inventions based on the gene expression profiles described herein. The data permits identification and quantification of gene expression products as biomarker of nutrition as well as disease prevention, identification and treatment of the underlying abnormal arthritic and gastrointestinal inflammatory disorders. Gene expression data elicited as a result of the practice of the methods of the invention also permits monitoring the progression of such abnormal musculoskeletal joint disorders. These inventions further include genetic testing to identify susceptible subpopulations of animals likely to be afflicted with such abnormal arthritic and gastrointestinal inflammatory disorders, to identify optimal diets for the prevention or treatment of such disorders, to identify pharmaceutical, nutraceutical and nutritional (dietary) interventions based on the findings set forth on this specification in order to treat the underlying diseases and inflammation. The inventions also include biomarker for early disease detection, targeted therapeutics, diagnostic reagents and kits for the analysis of tissue and blood samples from animals susceptible to or having such abnormal arthritic and gastrointestinal inflammatory disorders.

In designing foods for animals, for example, companion animals such as cats and dogs, optimal animal health or wellness through good nutrition is an important goal. However, even the most nutritious animal food is of little value if the animal rejects or refuses to eat the food, or if the animal's intake of the food is restricted because the animal finds the food unpalatable. Thus, the inventions set forth in this specification further comprise nutritional compositions capable of promoting the health and wellness of animals susceptible to or having such abnormal arthritic and gastrointestinal inflammatory disorders. The invention thus encompasses edible food compositions for companion animals, which have therapeutic and prophylactic efficacy and are palatable as compared with currently marketed companion animal food products.

### SUMMARY OF THE INVENTION

The invention generally encompasses pet food compositions for a companion animal that include an effective amount of ginger to prevent, to ameliorate the symptoms of, or to treat, inflammation or an inflammatory disorders in the companion animal, where the inflammation or inflammatory disorder is an arthritic condition or a gastrointestinal inflammatory disorder. The arthritic condition may be osteoarthritis, rheumatoid arthritis, psoriatic arthritis, ankylosing spondylitis or a local inflammatory joint condition. Gastrointestinal inflammatory disorders may include irritable bowel disorders and chronic diarrhea.

The pet food compositions of the invention containing ginger may also meet the nutritional requirements for a companion animal (e.g., an amount of protein, an amount of fat, an amount of carbohydrate, and/or an amount of fiber).

The present inventors have examined the effects of ginger in the diet of an animal. The animal may be a companion animal or livestock. The animal may be a dog. The animal may be a cat. Preferred companion animals of the invention are dogs and cats.

The animal may have a condition, disorder or symptom. The condition or disorder may be inflammatory. The condition may be arthritis. The disorder may be inflammatory bowel disorder. The condition or disorder may be a factor of age in the animal. The symptom may be diarrhea. The symptom may be swelling. The swelling may be at a joint in the animal.

In certain embodiments the invention encompasses pet food compositions for a companion animal that are nutritionally complete food compositions or diets for such animal For example, food compositions meeting the guidelines published in the Association of American Feed Control Officials Inc. (AAFCO) guidelines for nutritionally complete diets for canines or felines are encompassed by the invention.

In certain embodiments, the invention encompasses pet food compositions for a companion animal including at least about 0.5% by weight ginger. In other embodiments, the ginger may be present at about 1% by weight. In other embodiments, the ginger may be present at 1.5% by weight or may be present at about 2% by weight.

In other embodiments, the invention encompasses methods for improving the quality of life of a companion animal by feeding the animal a pet food composition comprising an amount of protein, an amount of fat, an amount of carbohydrate, an amount of fiber and an effective amount of ginger. In certain embodiments, the composition includes at least about 0.5% by weight ginger. In certain embodiments, the invention encompasses pet food compositions for a companion animal that include at least about 1% by weight ginger. In other embodiments, the ginger may be present at about 1.5% by weight or may be present at about 2% by weight.

In certain embodiments, the method encompasses feeding an animal in need thereof an amount of a composition including ginger, which is effective to enhance the animal's quality of life, wherein enhanced quality of life is evidenced by reduction in inflammatory arthritic conditions or gastrointestinal inflammatory disorders, or chronic diarrhea.

In other embodiments, the invention encompasses methods for preventing, ameliorating the symptoms of, or treating, an arthritic condition or a gastrointestinal inflammatory disorder or chronic diarrhea in a companion animal in need thereof comprising administering to the animal a pet food composition for a companion animal comprising an amount of protein, an amount of fat, an amount of carbohydrate, an amount of fiber and an effective amount of ginger to prevent, ameliorate the symptoms of, or treat, the arthritic condition or gastrointestinal inflammatory disorder or chronic diarrhea in the companion animal.

In other embodiments, the invention encompasses methods for preventing, ameliorating the symptoms of, or treating, an arthritic condition or a gastrointestinal inflammatory disorder or chronic diarrhea in a companion animal in need thereof, comprising administering to the animal a nutritionally complete diet for a companion animal comprising an amount of protein, an amount of fat, an amount of carbohydrate, an amount of fiber and an effective amount of ginger to prevent, ameliorate the symptoms of, or to treat the arthritic condition or a gastrointestinal inflammatory disorder or chronic diarrhea in the companion animal.

In certain embodiments, the arthritic condition may be osteoarthritis, rheumatoid arthritis, psoriatic arthritis, ankylosing spondylitis or a local inflammatory joint condition.

In another embodiment the gastrointestinal inflammatory disorder may be an inflammatory bowel disorder

In another embodiment the condition may be chronic diarrhea.

In a preferred embodiment the pet food composition is a nutritionally complete pet food diet for a canine or a feline.

In another embodiment, the invention encompasses a method for identifying one or more disease- or disorder-related genes in a companion animal, which one or more genes is differentially expressed by administering a pet food composition including ginger, comprising the steps of: (a) obtaining a sample of at least one RNA transcript or its translation product from the companion animal and comparing the number of RNA transcripts or translation products encoded by a particular gene or gene segment to the level found in a control companion animal. In certain embodiments, the gene may be any one included in Tables 10, 11, and 12 of the specification. In certain embodiments, the control companion animal may have the same disorder as the animal being tested or may not have such disorder.

In a particular embodiment, the invention encompasses detecting a change in an expression level of one or more genes or gene families associated with inflammation.

The invention also encompasses methods of screening for an agent comparable to ginger that modulates the differentiation of genes, comprising: preparing a first gene or gene family expression profile and/or assaying for an activity of a protein encoded by a gene or member of a gene family in a cell population; contacting the cell population with ginger; preparing a second gene or gene family expression profile and/or assaying for an activity of a protein encoded by a gene or member of a gene family of the cell population after being contacted with the agent; and comparing the first and second expression profiles and/or activities.

In another embodiment, the invention encompasses methods of monitoring the treatment of an animal on a diet, which includes compositions including ginger comprising administering a diet, which includes compositions including ginger to the animal, preparing a gene or gene family expression profile and/or assaying for an activity of a protein encoded by a gene or member of a gene family from a cell or tissue sample from the animal and comparing the expression profile and/or activity to an expression profile and/or activity from a sample collected prior to treatment or earlier in the treatment program.

Other and further objects, features, and advantages of the present invention will be readily apparent to those skilled in the art.

### BRIEF DESCRIPTION OF THE FIGURES

FIG. 1 illustrates the effects on inflammation and interleukin 1-beta of a control pet food composition and an illustrative pet food composition including ginger.

FIG. 2 illustrates the effects on inflammation and interleukin 6 of a control pet food composition and an illustrative pet food composition including ginger.

FIG. 3 illustrates the effects on collagen synthesis of a control pet food composition and an illustrative pet food composition including ginger.

FIG. 4 illustrates the percent change in synthesis: degradation ratio of collagen for a control pet food composition and an illustrative pet food composition including ginger.

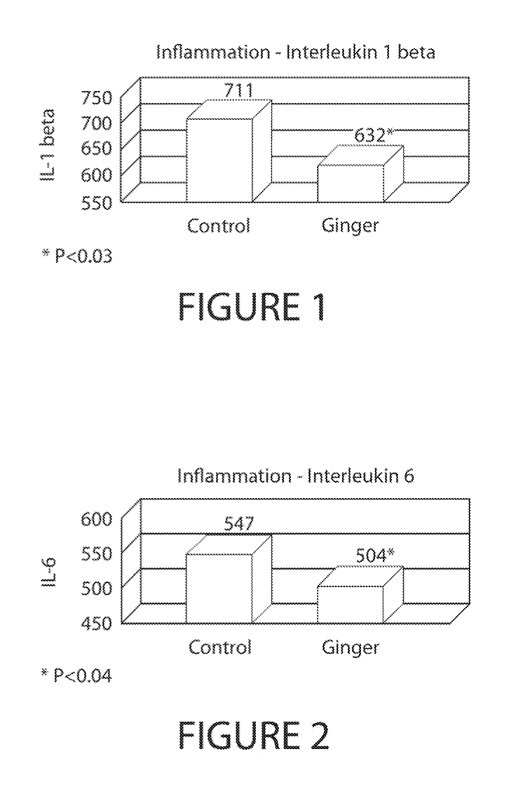

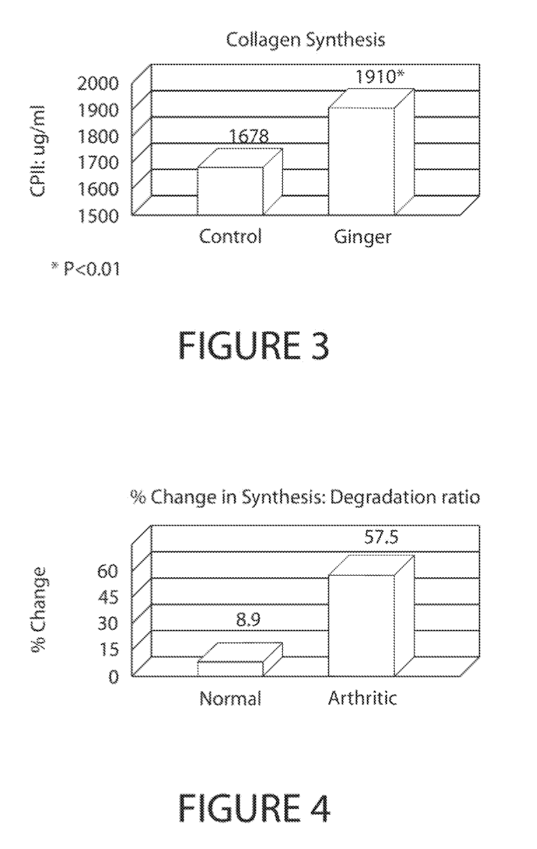

### DETAILED DESCRIPTION

It is contemplated that the invention described herein is not limited to the particular methodology, protocols, ingredients and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication, which might be used in connection with the invention.

As used herein and in the appended claims, the singular forms and “the” include plural reference unless the context clearly dictates otherwise.

The term “antibody” means any immunoglobulin that binds to a specific antigen, including IgG, IgM, IgA, IgD, and IgE antibodies. The term includes polyclonal, monoclonal, monovalent, humanized, heteroconjugate, antibody compositions with polyepitopic specificity, chimeric, bispecific antibodies, diabodies, single-chain antibodies and antibody fragments such as Fab, Fab′, F(ab′)₂, and Fv, or other antigen-binding fragments.

The term “antioxidant” means a substance that is capable of reacting with free radicals and neutralizing them. Illustrative examples of such substances include beta-carotene, selenium, coenzyme Q10 (ubiquinone), luetin, tocotrienols, soy isoflavones, S-adenosylmethionine, glutathione, taurine, N-acetylcysteine, vitamin E, vitamin C, lipoic acid and L-carnitine. Examples of foods containing useful levels of one or more antioxidants include but are not limited to ginkgo bilboa, green tea, broccoli, citrus pulp, grape pomace, tomato pomace, carrot spinach, and a wide variety of fruit meals and vegetable meals. It will be understood by one of skill in the art that while units of antioxidants may be provided herein as “ppm”, appropriate amounts of antioxidants may also be provided as “IU/kg” where appropriate and customary for a given antioxidant such as, e.g., Vitamin E.

The term “apoptosis” refers to one of the main types of programmed cell death and involves a series of biochemical events leading to a characteristic cell morphology and death, in more specific terms, a series of biochemical events that lead to a variety of morphological changes, including blebbing, changes to the cell membrane such as loss of membrane asymmetry and attachment, cell shrinkage, nuclear fragmentation, chromatin condensation, and chromosomal DNA fragmentation.

The term “array” means an ordered arrangement of at least two probes on a substrate. At least one of the probes is a control or standard and at least one of the probes is a diagnostic probe. The arrangement of from about two to about 40,000 probes on a substrate assures that the size and signal intensity of each labeled complex formed between a probe and a sample polynucleotide or polypeptide is individually distinguishable. The collection of molecules deposited on the array may be prepared either synthetically or biosynthetically. The array may take a variety of forms including libraries of soluble molecules, libraries of compounds tethered to resin beads, silica chips or other solid supports. The nucleic acid array may include libraries of nucleic acids which can be prepared by spotting nucleic acids in essentially any length (for example, from 1 to about 1,000 nucleotides in length) onto a substrate. A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, nylon, polystyrene, polycarbonate, plastics, glass, ceramic, stainless steel, or the like. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded.

The terms “beneficial change” in gene expression, or gene expression may be “beneficially altered” and like terms refer to a modification in gene expression (e.g., up or down regulation of mRNA levels) such that levels of proteins encoded by the genes may be correspondingly modified such that an associated biological pathway may be more likely to function normally and with less tendency to reflect pathological changes in the pathway that, e.g., may be typical of a senior animal. Generally, beneficial changes in gene expression relate to improved health and/or reduced propensity for disease in an animal. As used herein, measuring differences in gene expression “levels” and like terms refer to, e.g., characterizing whether expression of a gene is up or down regulated in an animal compared to a control level.

The term “biomarker” refers to genes and gene products encoded by a gene of the invention, wherein the gene has been determined to have been differentially expressed as a result of a disease, condition, disorder, or the administration of a substance, drug, nutrient or dietary component or combinations thereof, and wherein such genes and gene products of the invention are identified in Tables 10, 11 and 12. A biomarker may be a polynucleotide, polypeptide, protein, RNA, including an RNA transcript or its translation product, DNA, a metabolite of one or more of the foregoing molecules, or a useful variant of any one of the foregoing molecules, the differential expression of which is associated with an arthritic condition, a gastrointestinal inflammatory disorder or chronic diarrhea, and wherein the correlation of such differential expression in a sample taken from a test animal to a sample taken from a control animal can be used in the diagnosis, prognosis, monitoring or treatment of such arthritic condition, gastrointestinal inflammatory disorder or chronic diarrhea in an animal in need thereof. In addition, a biomarker can be generally used to refer to any portion or segment of such gene or protein that can identify or correlate with the full-length gene or protein, for example, in an assay or other method of the invention. Biomarker expression can also be identified by detection of biomarker translation (i.e., detection of biomarker protein in a sample). Methods suitable for the detection of biomarker protein include any suitable method for detecting and/or measuring proteins from a cell or cell extract. Such methods include but are not limited to, immunoblot (e.g., Western blot), enzyme-linked immunosorbent assay (ELISA), radioimmunoassay (RIA), immunoprecipitation, immunohistochemistry and immunofluorescence. Particularly preferred methods for detection of proteins include any single-cell assay, including immunohistochemistry and immunofluorescence assays. Such methods are well known in the art. Furthermore, antibodies against certain of the biomarker described herein are known in the art and are described in the public literature, and methods for their preparation are well known to the skilled worker.

The term “carbohydrate” as used herein includes polysaccharides (e.g., starches and dextrins) and sugars (e.g., sucrose, lactose, maltose, glucose, and fructose) that are metabolized for energy when hydrolyzed. Examples of carbohydrates suitable for inclusion in the compositions disclosed herein include, but are not limited to, corn, grain sorghum, wheat, barley, and rice.

The term “chronic diarrhea,” or “CD,” refers to a chronic or persistent presence of loose or liquid stool. Diarrhea may be caused by a viral, parasitic, or bacterial infection or it may be a symptom of another condition such as dysentery, cholera, botulism, lactose intolerance, appendicitis, celiac disease, fructose malabsorption, pernicious anemia, loss of pancreatic secretions, ulcerative colitis, ischemic bowel disease, bowel cancer, hormone tumors, or Crohn's disease. Diarrhea may be caused by dysfunction in the digestive system. Secretory diarrhea refers to an increase in the active secretion, or an inhibition of absorption. Osmotic diarrhea refers to too much water is drawn into the bowels, often by a shift in the concentration of salt ions such as sodium, potassium, and chloride. Motility-related diarrhea is caused by the rapid movement of food through the intestines (hypermotility). Inflammatory diarrhea occurs when there is damage to the mucosal lining or brush border, which leads to a passive loss of protein-rich fluids, and a decreased ability to absorb these lost fluids.

As used herein the term “compositions of the invention” refers to animal dietary food compositions including ginger. The compositions of the invention include ginger in an amount of about 0.5%, about 1%, about 1.5%, about 2%, about 3%, about 4%, about 5%, about 6%, about 7%, about 8%, about 9%, or about 10% by weight. The compositions of the invention may alleviate inflammatory bowel disorder. The compositions of the invention may alleviate diarrhea. The compositions of the invention may alleviate inflammation. The compositions of the invention may relieve arthritic conditions, for example, osteoarthritis, or less common inflammatory joint diseases in companion animals such as rheumatoid arthritis, psoriatic arthritis or other degenerative joint disorders. The compositions of the invention may alleviate cell apoptosis. The compositions of the invention may improve DNA repair. The compositions of the invention may alleviate protein damage. The compositions of the invention may alleviate oxidative stress.

The composition of the invention can be a liquid or a solid food. Where the composition is a liquid, the ginger can be admixed with other components. Where the composition is a solid, the ginger may be coated on the composition, incorporated into the composition, or both.

In various embodiments, the ginger may be added to the animal's food. In certain embodiments, the ginger may be added to the animal's food by a compounder or manufacturer at a site or by an animal's caregiver prior to feeding the animal. In other embodiments, the ginger may be added during the processing of an animal's food, such as during and/or after mixing of other components of the composition that is then packaged and made available to consumers. Such processing may include extrusion, canning, baking and the like or any other method of process of producing pet foods that is known in the art.

The terms “differentially expressed gene,” “differential gene expression,” “differential expression” or “differentially expressed” and their synonyms, which are used interchangeably, refer to a gene whose expression is activated to a higher or lower level in a subject suffering from a disease, condition, or disorder, or as a result of being administered a substance, drug, nutrient or dietary component or combinations thereof, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, condition, or disorder, or as a result of being administered a substance, drug, nutrient or dietary component or combinations thereof, or between various stages of the same disease, condition, or disorder, or as a result of being administered different amounts of a substance, drug, nutrient or dietary component or combinations thereof. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about 2.0, 1.9, 1.8, 1.7, 1.6, 1.5, 1.4, 1.3, 1.2, 1.1 or 1.0-fold change in the amount of transcribed messenger RNA or translated protein in a sample. In preferred embodiments of the invention, the differential expression is at least about 1.3 fold or greater, preferably at least two-fold, preferably at least about four-fold, more preferably at least about six-fold, most preferably at least about ten-fold difference between the expression of a given gene in subjects suffering from a disease, condition, or disorder, or as a result of the being administered a substance, drug, nutrient or dietary component or combinations thereof, or between various stages of the same disease, condition, or disorder, or as a result of the being administered different amounts of a substance, drug, nutrient or dietary component or combinations thereof.

The term “DNA repair” refers to a collection of processes by which a cell identifies and corrects damage to the DNA molecules that encode its genome. DNA repair ability of a cell is vital to the integrity of its genome and thus to its normal functioning and that of the organism. Both normal metabolic activities and environmental factors such as UV light and radiation can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome, which affect the survival of its daughter cells after it undergoes mitosis. Consequently, DNA repair processes are constantly active as it responds to damage in the DNA structure. The rate of DNA repair depends on many factors, such as the particular cell type, age of the cell, and the extracellular environment. A cell that has accumulated a large amount of DNA damage, or one that no longer effectively repairs damage incurred to its DNA, can enter one of three possible states: senescence, apoptosis, or unregulated cell growth.

The term “fold” when used as a measure of differential gene expression means an amount of gene expression in a canine that is a multiple or a fraction of gene expression compared to the amount of gene expression in a comparison canine, e.g., a canine having a loss of mobility or decreased mobility, compared to an animal not demonstrating such a condition. For example, a gene that is expressed 2 times as much in the animal as in the comparison animal has a 2-fold differential gene expression and a gene that is expressed one-third as much in the animal as in the comparison animal also has a 2-fold differential gene expression.

A “food” is a nutritionally complete diet for the intended recipient animal (e.g., domestic cat or domestic dog). The teen “fragment” means (1) an oligonucleotide or polynucleotide sequence that is a portion of a complete sequence and that has the same or similar activity for a particular use as the complete polynucleotide sequence or (2) a peptide or polypeptide sequence that is a portion of a complete sequence and that has the same or similar activity for a particular use as the complete polypeptide sequence. Such fragments can comprise any number of nucleotides or amino acids deemed suitable for a particular use. Generally, oligonucleotide or polynucleotide fragments contain at least about 10, 50, 100, or 1000 nucleotides and polypeptide fragments contain at least about 4, 10, 20, or 50 consecutive amino acids from the complete sequence. The term encompasses polynucleotides and polypeptides variants of the fragments. A polynucleotide, for example, can be broken up, or fragmented into, a plurality of segments.

Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.”), which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

The term “gene” or “genes” means a complete or partial segment of DNA involved in producing a polypeptide, including regions preceding and following the coding region (leader and trailer) and intervening sequences (introns) between individual coding segments (exons). The term encompasses any DNA sequence that hybridizes to the complement of gene coding sequences.

As used herein, the term “ginger” refers to the plant Zingiber officinale and derivatives thereof. By way of example, ginger may include products derived from the leaves, stems, seeds, and roots of a ginger plant. Ginger may be in the form of a ground powder, freshly ground, spray dried, freeze dried, wet root, extract, oil, suspension, oil and water, or solution. The ginger may be cooked or raw. An “extract” of ginger herein is any preparation containing substances extracted from ginger, including fluid extracts, tinctures, essential oils, distillates and oleoresins. Extracts of ginger may be prepared by any of the methods described herein or as known in the art by a skilled worker.

The term “homolog” means (1) a polynucleotide, including polynucleotides from the same or different animal species, having greater than 30%, 50%, 70%, 90%, 91%, 92%, 93%, 94%, 95%, 96%, 97%, 98% or 99% sequence similarity to a polynucleotide and having the same or substantially the same properties and performing the same or substantially the same function as the complete polynucleotide, or having the capability of specifically hybridizing to a polynucleotide under stringent conditions or (2) a polypeptide, including polypeptides from the same or different animal species, having greater than 30%, 50%, 70%, 90%, 91%, 92%, 93%, 94%, 95%, 96%, 97%, 98% or 99% sequence similarity to a polypeptide identified by the expression of polynucleotides and having the same or substantially the same properties and performing the same or substantially the same function as the complete polypeptide, or having the capability of specifically binding to a polypeptide identified by the expression of polynucleotides. Sequence similarity of two polypeptide sequences or of two polynucleotide sequences is determined using methods known to skilled artisans, e.g., the algorithm of Karlin and Altschul (Proc. Natl. Acad. Sci. USA 87:2264-2268 (1990)). Such an algorithm is incorporated into the NBLAST and XBLAST programs of Altschul et al. (J. Mol. Biol. 215:403-410 (1990)). To obtain gapped alignments for comparison purposes, Gapped BLAST can be utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://www.ncbi.nlm.nih.gov.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from nucleic acid arrays can be analyzed using commercially available software, such as those provided by Affymetrix or Agilent Technologies. Controls, such as for scan sensitivity, probe labeling and cDNA or cRNA quantization, are preferably included in the hybridization experiments. Hybridization signals can be scaled or normalized before being subject to further analysis. For instance, hybridization signals for each individual probe can be normalized to take into account variations in hybridization intensities when more than one array is used under similar test conditions. Hybridization signals can also be normalized using the intensities derived from internal normalization controls contained on each array. In addition, genes with relatively consistent expression levels across the samples can be used to normalize the expression levels of other genes. In one embodiment, probes for certain maintenance genes are included in a nucleic acid array of the present invention. These genes are chosen because they show stable levels of expression across a diverse set of tissues. Hybridization signals can be normalized and/or scaled based on the expression levels of these maintenance genes.

The term “hybridization complex” means a complex that is formed between sample polynucleotides when the purines of one polynucleotide hydrogen bond with the pyrimidines of the complementary polynucleotide, e.g., 5′-A-G-T-C-3′ base pairs with 3′-T-C-A-G-5′. The degree of complementarily and the use of nucleotide analogs affect the efficiency and stringency of hybridization reactions.

The term “hybridization probes” includes nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

As used herein, “improving” or “enhancing” the quality of life of an animal refers to an improvement or enhancement in one or more characteristics selected from a group consisting of alertness, vitality, protection of cartilage, maintenance of muscle mass, digestibility, and skin and pelage quality. Additionally, improvement/enhancement in blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway and electron transport are also contemplated.

An “improvement” or an “enhancement” in a characteristic or biological pathway refers to a modification in said characteristic or biological pathway such that there is a tendency for the characteristic or pathway to appear and/or function normally and with less tendency to reflect pathological changes in the characteristic or pathway that, e.g., may be typical of a senior animal.

The term “inflammation” refers to a protective attempt by an organism to remove an injurious stimulus and initiate the healing processes for the tissue affected by the injurious stimulus. Inflammation can be classified as either acute or chronic. Acute inflammation is an initial response to harmful stimuli and is achieved by the increased movement of plasma and leukocytes from the blood into the injured tissues. A cascade of biochemical events propagates and matures the inflammatory response, involving the local vasculature, the immune system, and various cells within the injured tissue. Chronic inflammation, or prolonged inflammation, leads to a progressive shift in the type of cells, which are present at the site of inflammation, and is characterized by simultaneous destruction and healing of the tissue from the inflammatory process.

The terms “inflammatory bowel disease” or “IBD” refer to an inflammatory condition of the large and sometimes small intestines. The terms also refer to a group of idiopathic gastrointestinal disorders characterized by continuous or recurring abdominal pain or cramping. The pain may range from mild to severe. Pain is frequently associated with altered bowel motility (e.g., diarrhea, constipation or both).

As used herein, an “ingredient” refers to any component of a composition.

The term “large breed” canine means a canine that generally weighs more than 55 pounds when an adult. The term “regular breed” canine means a canine that weighs less than 55 pounds and more than 20 pounds when an adult. The term “small breed” canine means a canine that weighs less than 20 pounds when an adult. The terms “senior” or “mature adult” refers to the life-stage of an animal. For small or regular breed canines, the “senior” life stage is from about 7 to about 10 years of age. For felines, the “senior” life stage is from about 7 to about 12 years of age. For large breed canines, over 5 years of age represents “senior.”

The term “nutrient” refers to a substance that provides nourishment. In some cases an ingredient may comprise more than one “nutrient,” for example, a composition may comprise fish oil as an ingredient, the oil itself comprising important nutrients such as eicosapentaenoic acid and docosahexaenoic acid. The distinction in these terms is familiar to one of skill in the art.

As contemplated herein, the compositions of the present invention are meant to encompass nutritionally complete and balanced animal feed compositions that additionally comprise ginger. A “nutritionally complete diet” is a diet that includes sufficient nutrients for maintenance of normal health of a healthy animal on the diet.

Nutritionally complete and balanced pet food compositions are familiar to one of skill in the art. For example substances such as nutrients and ingredients suitable for nutritionally complete and balanced animal feed compositions, and recommended amounts thereof, may be found for example, in the Official Publication of the Association of American Feed Control Officials, Inc. (AAFCO), Atlanta, Ga., 2005, or the National Research Council's Nutrient Requirements of Dogs and Cats, The National Academy Press, Washington, D.C., 2006.

For example, a nutritionally complete and balanced dog food composition of the present invention may comprise: about 0 to about 90%, preferably about 5% to 45%, by weight of carbohydrates; about 5% to about 70%, preferably about 10% to about 60%, by weight of protein; about 2% to about 50%, preferably about 5% to about 40%, by weight of fat; about 0.1% to about 20%, preferably about 1% to about 11%, by weight of total dietary fiber; about 0 to about 15%, preferably about 2% to about 8%, by weight of vitamins and minerals, antioxidants, and other nutrients which support the nutritional needs of the animal; and about 0.5% to about 1.5%, preferably 1%, by weight of ginger.

For example, a nutritionally complete and balanced cat food composition of the present invention may comprise: about 0 to about 90%, preferably about 5% to 45%, by weight of carbohydrates; about 5% to about 70%, preferably about 20% to about 60%, by weight of protein; about 2% to about 50%, preferably about 5% to about 40%, by weight of fat; about 0.1% to about 20%, preferably about 1% to about 11%, by weight of total dietary fiber; about 0 to about 15%, preferably about 2% to about 8%, by weight of vitamins and minerals, antioxidants, and other nutrients which support the nutritional needs of the animal; and about 0.5% to about 1.5%, preferably 1%, by weight of ginger.

The term “oxidative stress” refers to an imbalance between the production of reactive oxygen species and a biological system's ability to readily detoxify the reactive intermediates or easily repair the resulting damage. Reactive oxygen species include super oxide, ozone, peroxide and a hydroxyl radical. Oxidative stress causes cellular damage through mechanisms such as damage of DNA, oxidations of polydesaturated fatty acids in lipids, oxidations of amino acids in proteins, and oxidatively inactivate specific enzymes by oxidation of co-factors.

The term “polynucleotide” or “oligonucleotide” means a polymer of nucleotides. The term encompasses DNA and RNA (including cDNA and mRNA) molecules, either single or double stranded and, if single stranded, its complementary sequence in either linear or circular form. The term also encompasses fragments, variants, homologs, and alleles, as appropriate for the sequences that have the same or substantially the same properties and perform the same or substantially the same function as the original sequence. The sequences may be fully complementary (no mismatches) when aligned or may have up to about a 30% sequence mismatch. Preferably, for polynucleotides, the chain contains from about 50 to 10,000 nucleotides, more preferably from about 150 to 3,500 nucleotides. Preferably, for oligonucleotides, the chain contains from about 2 to 100 nucleotides, more preferably from about 6 to 30 nucleotides. The exact size of a polynucleotide or oligonucleotide will depend on various factors and on the particular application and use of the polynucleotide or oligonucleotide. The term includes nucleotide polymers that are synthesized and that are isolated and purified from natural sources. The term “polynucleotide” is inclusive of “oligonucleotide.”

The term “polypeptide,” “peptide,” or “protein” means a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompass polymers of any length, preferably polymers containing from about 2 to 1000 amino acids, more preferably from about 5 to 500 amino acids. The term includes amino acid polymers that are synthesized and that are isolated and purified from natural sources.

The term “probe” means (1) an oligonucleotide or polynucleotide, either RNA or DNA, whether occurring naturally as in a purified restriction enzyme digest or produced synthetically, that is capable of annealing with or specifically hybridizing to a polynucleotide with sequences complementary to the probe or (2) a peptide or polypeptide capable of specifically binding a particular protein or protein fragment to the substantial exclusion of other proteins or protein fragments. An oligonucleotide or polynucleotide probe may be either single or double stranded. The exact length of the probe will depend upon many factors, including temperature, source, and use. For example, for diagnostic applications, depending on the complexity of the target sequence, an oligonucleotide probe typically contains about 10 to 100, to 50, or 15 to 25 nucleotides. In certain diagnostic applications, a polynucleotide probe contains about 100-1000, 300-600, nucleotides, preferably about 300 nucleotides. The probes herein are selected to be “substantially” complementary to different strands of a particular target sequence. This means that the probes must be sufficiently complementary to specifically hybridize or anneal with their respective target sequences under a set of predetermined conditions. Therefore, the probe sequence need not reflect the exact complementary sequence of the target. For example, a noncomplementary nucleotide fragment may be attached to the 5′ or 3′ end of the probe, with the remainder of the probe sequence being complementary to the target sequence. Alternatively, noncomplementary bases or longer sequences can be interspersed into the probe provided that the probe sequence has sufficient complementarity with the sequence of the target polynucleotide to specifically anneal to the target polynucleotide. A peptide or polypeptide probe may be any molecule to which the protein or peptide specifically binds, including DNA (for DNA binding proteins), antibodies, cell membrane receptors, peptides, cofactors, lectins, sugars, polysaccharides, cells, cell membranes, organelles and organellar membranes.

The term “RNA transcript” includes, but is not limited to, pre-mRNA transcripts, transcript processing intermediates, mature RNA transcripts ready for translation and transcripts of the gene or genes, or nucleic acids derived from the RNA transcripts. Transcript processing may include splicing, editing, and degradations. As used herein, a nucleic acid derived from an RNA transcript refers to a nucleic acid for whose synthesis the RNA transcript or a subsequence thereof has ultimately served as a template. Thus, a cDNA transcribed from an mRNA, a cRNA transcribed from that cDNA, a DNA amplified from the cDNA, an RNA transcribed from the amplified DNA, etc., are all derived from the RNA transcript and detection of such derived products is indicative of the presence and/or abundance of the original transcript in a sample. Thus, mRNA derived samples comprising RNA transcripts include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The terms “sample” and “specimen” mean any animal tissue or fluid containing, e.g., polynucleotides, polypeptides, antibodies, metabolites, and the like, including cells and other tissue containing DNA and RNA. Examples include: blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

As used herein, “soluble fiber” refers to dietary fiber that attracts water during digestion and slows the rate of nutrient absorption and is typically found in, e.g. oat bran, seeds, beans, and certain fruits and vegetables such as beet pulp, guar gum, chicory root, psyllium, pectin, blueberry, cranberry, squash, apples, oats, beans, citrus, barley and peas. As used herein, the term encompasses any source of soluble fiber suitable for the compositions disclosed herein as would be evident to one of skill in the art.

The term “specifically bind” means a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

The term “specifically hybridize” means an association between two single stranded polynucleotides of sufficiently complementary sequence to permit such hybridization under predetermined conditions generally used in the art (sometimes termed “substantially complementary”). For example, the term may refer to hybridization of a polynucleotide probe with a substantially complementary sequence contained within a single stranded DNA or RNA molecule according to an aspect of the invention, to the substantial exclusion of hybridization of the polynucleotide probe with single stranded polynucleotides of non-complementary sequence.

The term “stringent conditions” means (1) hybridization in 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., (2) hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C.; with washes at 42° C. in 0.2×SSC and 0.1% SDS or washes with 0.015 M NaCl, 0.0015 M sodium citrate, 0.1% Na₂SO₄ at 50° C. or similar procedures employing similar low ionic strength and high temperature washing agents and similar denaturing agents.

The term “substance” means an element, compound, molecule, or a mixture thereof or any other material that could potentially be useful for diagnosing, prognosing, or modulating the onset or severity of an abnormal joint condition in an animal, including any drug, chemical entity, or biologic entity.

As used herein, the “supplement(s)” include, but are not limited to, a feed used with another feed to improve nutritive balance or performance of the total. Supplements include, but are not limited to, compositions that are fed undiluted as a supplement to other feeds, offered free choice with other parts of an animal's ration that are separately available, or diluted and mixed with an animal's regular feed to produce a complete feed. The AAFCO guidelines, for example, contain a discussion relating to supplements in the Association of American Feed Control Officials Inc. Official Publication. Supplements may be in various forms including, for example, powders, liquids, syrups, pills, encapsulated compositions, and the like.

The term “useful variations” means (1) for a polynucleotide, the complements of the polynucleotide; the homologs of the polynucleotide and its complements; the variants of the polynucleotide, its complements, and its homologs; and the fragments of the polynucleotide, its complements, its homologs, and its variants and (2) for a polypeptide, the homologs of the polypeptide; the variants of the polypeptide and its homologs; and the fragments of the polynucleotide, its homologs, and its variants.

The term “variant” means (1) a polynucleotide sequence containing any substitution, variation, modification, replacement, deletion, or addition of one or more nucleotides from or to a polynucleotide sequence and that has the same or substantially the same properties and performs the same or substantially the same function as the original sequence and (2) a polypeptide sequence containing any substitution, variation, modification, replacement, deletion, or addition of one or more amino acids from or to a polypeptide sequence and that has the same or substantially the same properties and performs the same or substantially the same function as the original sequence. The term therefore includes single nucleotide polymorphisms (SNPs) and allelic variants and includes conservative and non-conservative amino acid substitutions in polypeptides. The term also encompasses chemical derivatization of a polynucleotide or polypeptide and substitution of nucleotides or amino acids with nucleotides or amino acids that do not occur naturally, as appropriate.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes. Changes in gene expression also are associated with pathogenesis. For example, the lack of sufficient expression of functional tumor suppressor genes and/or the over expression of oncogene/protooncogenes could lead to tumor genesis or hyperplastic growth of cells (Marshall, (1991) Cell 64:313-326; Weinberg, (1991) Science 254:1138-1146). Thus, changes in the expression levels of particular genes (e.g., oncogenes or tumor suppressors) serve as signposts for the presence and progression of various diseases. Monitoring changes in gene expression may provide certain advantages during disease progression or disease treatment. Often agents are screened for the ability to interact with a major target without regard to other effects on cells. Often such other effects cause toxicity in the whole animal, which prevent the development and use of the potential drug.

In practicing the present invention, many conventional techniques in molecular biology may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The expression profiles have been used to identify individual genes that are differentially expressed under one or more conditions. In addition, the present invention identifies families of genes that are differentially expressed. As used herein, “gene families” includes, but is not limited to, the specific genes identified by accession number herein, as well as related sequences. Related sequences may be, for example, sequences having a high degree of sequence identity with a specifically identified sequence either at the nucleotide level or at the level of amino acids of the encoded polypeptide. A high degree of sequence identity is seen to be at least about 65% sequence identity at the nucleotide level to said genes, about 80 or 85% sequence identity or about 90 or 95% or more sequence identity to said genes. With regard to amino acid identity of encoded polypeptides, a high degree of identity is seen to be at least about 50% identity, about 75% identity and about 85% or more sequence identity. In particular, related sequences include homologous genes from different organisms. For example, if the specifically identified gene is from a non-human mammal, the gene family would encompass homologous genes from other mammals including humans. If the specifically identified gene is a human gene, gene family would encompass the homologous gene from different organisms. Those skilled in the art will appreciate that a homologous gene may be of different length and may comprise regions with differing amounts of sequence identity to a specifically identified sequence.

CUBN, or Cubilin precursor, refers to both a protein of approximately 3600 amino acids in length with a mass of around 40 kDa and the gene that encodes it. The CUBN protein comprises a subunit that interacts with LRP2 in a dual-receptor complex in a calcium-dependent manner. The CUBN protein is a component of the cubam complex composed of CUBN and AMN. The CUBN protein may be found in a complex with PID1/PCLI1, LRP1 and CUBNI. The CUBN protein may be located subcellularly at the endosome membrane and in the endocytotic apparatus of epithelial cells. The CUBN protein may be N-glycosylated. Cubilin is important for Vitamin B12 absorption and for reabsorption of proteins. It is essential for renal tubular uptake of albumin. Cubilin serves as receptor for intrinsic factor B12 complex. Dysfunctional Cubilin affects cellular uptake and metabolism of steroid hormones (e.g., vitamin D3). In certain embodiments, cubilin may be modulated by ginger in the diet. In other embodiments, cubilin may be up-regulated by ginger in the diet.

TLR, or toll-like receptor, refers to a protein and the gene encoding it, wherein the increased presence of the TLR protein upregulates the cytokines IFN-alpha (interferon=IFN), IFN-beta, IL-28 (IL=interleukin), and IL-29.

Folate receptor (FOLR) refers to a family of receptors for the necessary nucleotide precursor ingredient folate and the genes that encode them. Members of this gene family have a high affinity for folic acid and for several reduced folic acid derivatives, and mediate delivery of 5-methyltetrahydrofolate to the interior of cells.

Fibronectin precursor, or FBN, refers to a gene and expressed protein of approximately 2400 amino acids with a mass of around 262 kDa. FBN may assemble as homodimers, heterodimers or multimers of alternatively spliced variants. They may be connected by disulfide bonds near the carboxyl ends. They may interact with FBLN1, AMBP, TNR, LGALS3BP and COL13A1. FBN may be secreted and found in the extracellular space or extracellular matrix. FBN may be post-translationally modified through sulfation or phosphorylation.

Talin refers to a high-molecular weight cytoskeletal protein concentrated at region of cell-substratum contact and the gene encoding it. It links actin cytoskeleton to extracellular environment. With reduced talin there will be less cell to cell contact in oocytes, integrin signaling will be inhibited, and cells will be less invasive, i.e., talin may have some tumor suppression effects, e.g., through ADP-ribose pyrophosphatase (mitochondrial precursor). Talin may bind manganese or magnesium as a cofactor. Talin may locate at or near mitochondria. Talin may interact with C17 or f25. In certain embodiments, talin may be modulated by ginger in the diet. In other embodiments, talin may be down-regulated by ginger in the diet.

Bcl-2 refers to an apoptosis related signaling protein. Bcl-2 induces apoptosis, is involved in cell arrest and appears to have tumor suppressor activity. In certain embodiments, Bcl-2 associated transcription factor (BCLAFI orbit) may be modulated by ginger in the diet. In other embodiments, Bcl-2 may be up-regulated by ginger in the diet.

Stress-70 protein (HSPA9B) refers to a gene and protein that controls cell proliferation and cellular aging. In certain embodiments, HSPA9B may be modulated by ginger in the diet. In other embodiments, HSPA9B may be up-regulated by ginger in the diet.

Caspases refer to the genes and proteins involved in apoptosis signaling. Caspase 6 is involved in the activation cascade of caspases responsible for apoptosis execution. Caspase 3 activates caspase 6. Over-expression of caspases promotes programmed cell death. In certain embodiments, Caspases may be modulated by ginger in the diet. In other embodiments, Caspases may be up-regulated by ginger in the diet. In other embodiments, Caspase 3 and 6 may be up-regulated by ginger in the diet.

Ribonucleoside-diphosphate reductase M2 subunit B (RRM2B) is involved in DNA repair. It supplies deoxyribonucleotides for DNA repair in cells arrested at GI or G2. The gene is induced by p53.

CARF (collaborates/cooperates with ARF (alternate reading frame)) refers to a gene and protein that enhances p53 functions. CARF may exert a vital role on p53-HDM2-p2l (WAFI) pathway that is central to cell cycle control, senescence, and DNA damage response of human cells. CARF may be modulated by ginger in the diet. In certain embodiments, CARF may be up-regulated by ginger in the diet.

Insulin-like growth factor-binding protein 6 precursor (IGFBP6) refers to a gene and protein wherein the protein binds and inhibits IGF-1 and IGF-2 and alters their interaction with surface receptors. Binding of IBP to IGF decreases cell survival and enhances apoptosis. In certain embodiments, IGFBP6 may be modulated by ginger in the diet. In certain embodiments, IGFBP6 may be up-regulated by ginger in the diet.

Transcobalamin (TCN I) is a vitamin B12 binding protein and gene. It facilitates the transport of cobalamin (vitamin B12) into cells. In certain embodiments, TCN I may be modulated by ginger in the diet. In certain embodiments, TCN I may be up-regulated by ginger in the diet.

Retinol binding protein 4 (RBP4) refers to a gene and a protein wherein the protein binds and transports retinol. In certain embodiments, RBP4 may be modulated by ginger in the diet. In certain embodiments, RBP4 may be up-regulated by ginger in the diet.

Inducible T-cell co-stimulator precursor (ICOS) refers to a gene and a protein that plays a role in development of cell help for B cells and in the generation, survival, and reactivation of memory CD4 T cells and B cells. In certain embodiments, ICOS may be modulated by the presence of ginger in the diet. In certain embodiments, ICOS may be up-regulated by ginger in the diet.

Topoisomerase (TOP2A) refers to a gene and protein wherein the protein is an enzyme involved in DNA replication. It functions as a target for several anticancer agents. In certain embodiments, it may be modulated by the presence of ginger in the diet. In certain embodiments, it may be down-regulated by ginger in the diet.

DNA Primase is involved in DNA replication. In certain embodiments, DNA Primase (PRIM1) may be modulated by the presence of ginger in the diet. In certain embodiments, it may be down-regulated by ginger in the diet.

Interleukin-1 receptor type 11 (IL1-R2) refers to a gene and a protein wherein the protein is a receptor for IL-1. This IL-1 receptor does not contribute to IL-1 signaling. It acts as a decoy for IL-1 and inhibits IL-1 activity. This is a specific down-regulator of IL-1 action. In certain embodiments, ILI-R2 may be modulated by the presence of ginger in the diet. In certain embodiments, it may be up-regulated by the presence of ginger in the diet.

Multisynthetase complex auxiliary component p43 contains endothelial monocyte-activating polypeptide 2 (EMAPII or SCYE) and refers to a gene and a protein formed by EMAPII. The EMAPII product has inflammatory cytokine activity. It is induced by apoptosis. In certain embodiments, it may be modulated by the presence of ginger in the diet. In certain embodiments, it may be down-regulated with ginger in the diet.

Calreticulin precursor (CALR) refers to a gene and the resulting protein wherein the gene product is a Calcium binding/storage protein located in the ER and in nucleus. CALR can inhibit the binding of glucocorticoid receptor, androgen receptors to their response elements. CALR acts as an important modulator of regulation of gene transcription by nuclear hormone receptors. This gene is up-regulated in colorectal cancer and liver cancer. Calreticulin is increased on the surface of apoptotic cells and interacts with adiponectin during clearance of apoptotic cells. It may be modulated by the presence of ginger in the diet. It may be down-regulated by the presence of ginger in the diet.

Natural cytotoxicitytriggering receptor 3 (NCR3) refers to a gene and encoded protein that is up-regulated in rheumatoid arthritis, with LPS, INF-gamma, or bacterial infection. In certain embodiments, it may be modulated by the presence of ginger in the diet. In other embodiments, it may be down-regulated with ginger in the diet.

Interleukin-18 binding protein precursor (IL-18BP) refers to a gene and encoded protein wherein the protein IL-18BP binds IL-18 and inhibits its functions. It is an inhibitor of early TH1 cytokine response. It inhibits IL-18-induced INF-gamma production. It has been shown to have anti-inflammatory effect in skin inflammation. In certain embodiments, it may be modulated by the presence of ginger in the diet. In other embodiments, it may be up-regulated with ginger in the diet.

Prolyl 4-hydroxylase alpha-1 and alpha-2 subunit precursor (P4HA) refers to a gene and resulting protein wherein the protein in an enzyme and the active enzyme is a tetramer of 2 alpha and 3 beta subunits. This enzyme catalyzes post-translational formation of 4-hydroxyproline in collagen and other proteins. It has pro collagen-proline 4-dioxygenase activity. In certain embodiments, it may be modulated by the presence of ginger in the diet. In other embodiments, it may be up-regulated with ginger in the diet.

Baculoviral IAP repeat-containing protein 3 (BIRC3: c-IAP2) refers to a gene and protein wherein the gene product acts as an apoptotic suppressor. In certain embodiments, it may be modulated by the presence of ginger in the diet. In other embodiments, it may be up-regulated with ginger in the diet.

Growth arrest and DNA-damage-inducible protein 34 (GADD34), regulatory (inhibitor) subunit 15A (PPPIR15A) refers to a gene that is a member of a group of genes whose transcript levels are increased following stressful growth arrest conditions, treatment with DNA-damaging agents, heat shock, nutrient deprivation, energy depletion, and ER stress. It inhibits viral replication via inhibition of the mTOR pathway. GADD34 is a protein that plays a pivotal role in the recovery of cells from shut-down of protein translation induced by ER stress. GADD34 contributes to the regulation of p21 expression. It suppresses cellular proliferation through the induction of cellular senescence. In certain embodiments, it may be modulated by the presence of ginger in the diet. In other embodiments, it may be up-regulated with ginger in the diet.

WW domain-containing oxidoreductase (WWOX) refers to a probable oxidoreductase, which may act as a tumor suppressor and plays a role in apoptosis and may function synergistically with p53 to control genotoxic stress-induced cell death. WWOX may mediate TNF-alpha-induced apoptosis. In certain embodiments, it may be modulated by the presence of ginger in the diet. In other embodiments, it may be down-regulated with ginger in the diet.

Ring finger protein 139 (TRCB=RNF139) refers to a gene and protein wherein the gene is a potential tumor suppressor gene for renal cell carcinoma. The gene product is located at the ER and has been shown to possess ubiquitin ligase activity. The protein may interact with the tumor suppressor protein von Hippel-Lindau (VHL). Expression of TRC8 represses genes involved in cholesterol and fatty acid biosynthesis that are transcriptionally regulated by the sterol response element binding proteins (SREBPs). In certain embodiments, it may be modulated by the presence of ginger in the diet. In other embodiments, it may be up-regulated with ginger in the diet.

As used herein, methods to “treat” an animal suffering from a disease or disorder is also meant to encompass methods to prevent and/or to ameliorate the disease or disorder as well.

The invention generally encompasses pet food compositions for a companion animal comprising an amount of protein, an amount of fat, an amount of carbohydrate, an amount of fiber and an effective amount of ginger to prevent inflammation or an inflammatory disorder in a companion animal.

In certain embodiments, the effective amount of ginger is at least 0.5% by weight ginger. In other embodiments of the invention ginger is present in a range of about 0.5% to about 1.5% by weight on a dry matter basis.

In certain embodiments, the companion animal is a dog.

In certain embodiments, the companion animal is a cat.

The compositions in addition to the ginger include at least one component suitable for consumption by a companion animal including, but not limited to, fats, carbohydrates, proteins, fibers, nutritional balancing agents such as vitamins, minerals, and trace elements, and mixtures thereof. One of ordinary skill in the art can select the amount and type of food ingredients for a typical food based upon the dietary requirements of the animal, for example, the animal's species, age, size, weight, health, and function.

The food ingredient part of the food composition can include up to about 100% of any particular food ingredient or can include a mixture of food ingredients in various proportions. In certain embodiments, the food composition includes a combination of food ingredients in amounts of about 0 wt. % to 50 wt. % fat, 0 wt. % to 75 wt. % carbohydrate, 0 wt. % to 95 wt. % protein, 0 wt. % to 40 wt. % dietary fiber, and 0 wt. % to 15 wt. % of one or more nutritional balancing agents.

In certain embodiments, the fat and carbohydrate food ingredient is obtained from a variety of sources such as animal fat, fish oil, vegetable oil, meat, meat by-products, grains, other animal or plant sources, and mixtures thereof. Grains include wheat, corn, barley, and rice.

In certain embodiments, the protein food ingredient is obtained from a variety sources such as plants, animals, or both. Animal protein includes meat, meat by-products, dairy, and eggs. Meats include the flesh from poultry, fish, and animals such as cattle, swine, sheep, goats, and the like; meat by-products include lungs, kidneys, brain, livers, stomachs, and intestines. The protein food ingredient may also be free amino acids and/or peptides. Preferably, the protein food ingredient includes meat, a meat by-product, dairy products, or eggs.

In certain embodiments, the fiber food ingredient is obtained from a variety of sources such as vegetable fiber sources, for example, cellulose, beet pulp, peanut hulls, and soy fiber.

In certain embodiments, the nutritional balancing agents are obtained from a variety of sources known to skilled artisans, for example, vitamin and mineral supplements and food ingredients. Vitamins and minerals can be included in amounts required to avoid deficiency and maintain health. These amounts are readily available in the art. The National Research Council (NRC) provides recommended amounts of such nutrients for farm animals. See, e.g., Nutrient Requirements of Swine (10th Rev. Ed., Nat'l Academy Press, Wash. D.C., 1998), Nutrient Requirements of Poultry (9th Rev. Ed., Nat'l Academy Press, Wash. D.C., 1994), Nutrient Requirements of Horses (5th Rev. Ed., Nat'l Academy Press, Wash. D.C., 1989). The Association of American Feed Control Officials Inc. (AAFCO) provides recommended amounts of such nutrients for dogs and cats. See American Feed Control Officials, Inc., Official Publication, pp. 137-152 (2009). Vitamins generally useful as food additives include vitamin A, vitamin B1, vitamin B2, vitamin B6, vitamin B12, vitamin D, biotin, vitamin K, folic acid, inositol, niacin, and pantothenic acid. Minerals and trace elements useful as food additives include calcium, phosphorus, sodium, potassium, magnesium, copper, zinc, chloride, iron, selenium, iodine, and iron.

In certain embodiments, the food compositions may contain additional ingredients such as vitamins, minerals, fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, sex, and diet; the animal's consumption rate; the type of disease or condition being treated; and the like. Therefore, the component and ingredient amounts may vary widely and may deviate from the preferred proportions described herein.

In certain embodiments, the amount of protein is 5 wt. % to 70 wt. % based on the total weight of the composition.

In certain embodiments, the amount of fat is 2 wt. % to 50 wt. % based on the total weight of the composition.

In certain embodiments, the amount of fiber is 0.1 wt. % to 20 wt. % based on the total weight of the composition.

In certain embodiments, the amount of carbohydrate is 0 wt. % to 90 wt. % based on the total weight of the composition.

In certain embodiments, the pet food composition further comprises any of the following vitamin E, eicosapentaenoic acid (EPA), docosahexaenoic acid (DHA), other n-3 fatty acids, n-6 fatty acids, crude fiber, soluble fiber, and mixtures of the foregoing ingredients.

In certain embodiments, the ginger is present at about 0.5% by weight.

In certain embodiments, the ginger is present at about 1% by weight.

In certain embodiments, the ginger is present at about 1.5% by weight.

In certain embodiments, the ginger is present at about 2% by weight.

The compositions can contain additional ingredients intended to maintain or improve the health of the animal, for example, supplements, medications, herbs, holistic drugs and compositions, and the like.

The composition of the invention may include one or more additional ingredients to prevent or treat one or more diseases or conditions. The component in the diet which accomplishes this may be an antioxidant or mixture thereof. An antioxidant is a material that quenches a free radical. Examples of such materials include foods such as Ginkgo Biloba, citrus pulp, grape pomace, tomato pomace, carrot and spinach, all preferably dried as well as various other materials such as beta-carotene, selenium, coenzyme Q10 (ubiquinone), lutein, tocotrienols, soy isoflavones, S-adenosylmethionine, glutathione, taurine, N-acetylcysteine, Vitamin E, Vitamin C, alpha-lipoic acid, 1-carnitine and the like. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The d form is preferable but racemic mixtures are acceptable. The forms and derivatives will function in a Vitamin E like activity after ingestion by the pet. Vitamin C can be administered in this diet as ascorbic acid and its various derivatives thereof such as calcium phosphate salts, cholesteryl salt, 2-monophosphate, and the like which will function in a vitamin C like activity after ingesting by the pet. They can be in any form such as liquid, semisolid, solid and heat stable form. L-carnitine can be administered in the diet and various derivatives of carnitine such as the salts such as the hydrochloride, fumarate and succinates, as well as acetylated carnitine, and the like can be used.

The quantities administered in the diet, all as wt % (dry matter basis) of the diet, are calculated as the active material, per se, that is measured as free material. The maximum amounts employed should not bring about toxicity. At least about 100 ppm or at least about 150 ppm of Vitamin E can be used. A preferred range of 500 to 1,000 ppm can be employed. Although not necessary, a maximum of about 2000 ppm or about 1500 ppm is generally not exceeded. With respect to Vitamin C, at least about 50 ppm is used, desirably at least about 75 ppm and more desirably at least about 100 ppm. A non-toxic maximum can be employed. The quantity of lipoic acid can vary from at least about 25, desirably at least about 50 ppm, more desirably about 100 ppm. Maximum quantities can vary from 100 ppm to 600 ppm or to an amount which remains non-toxic to the pet. A preferred range is from 100 ppm to 200 ppm. For 1-carnitine about 50 ppm, desirably about 200 ppm, more desirably about 300 ppm for canines are a useful minimum. For felines, slightly higher minimums of 1-carnitine can be employed such as about 100 ppm, 200 ppm, and 500 ppm. A non-toxic maximum quantity can be employed, for example, less than about 5,000 ppm. For canines, lower quantities can be employed, for example, less than about 5,000 ppm. For canines, a preferred range is 200 ppm to 400 ppm. For felines, a preferred range is 400 ppm to 600 ppm. Beta-carotene at 1-15 ppm can be employed. Selenium at 0.1 up to 5 ppm can be employed. Lutein at least about 5 ppm can be employed. Tocotrienols at least about 25 ppm can be employed. Coenzyme Q10 at least about 25 ppm can be employed. S-adenosylmethionine at least about 50 ppm can be employed. Taurine at least about 1000 ppm can be employed. Soy isoflavones at least about 25 ppm can be used. N-acetylcysteine at least about 50 ppm can be used. Glutathione at least about 50 ppm can be used. Gingko Biloba at least 50 ppm of extract can be used.

In certain embodiments, the compositions further include an effective amount of at least one substance selected from the group consisting of glucosamine, chondroitin, chondroitin sulfate, methylsulfonylmethane (“MSM”), creatine, antioxidants, Perna canaliculata, omega-3 fatty acids, omega-6 fatty acids and mixtures thereof.

In certain embodiments, the composition can be a treat. Treats include compositions that are given to an animal to entice the animal to eat during a non-meal time, for example, dog bones for canines. Treats may be nutritional wherein the composition includes one or more nutrients or and may have a food-like composition. Non-nutritional treats encompass any other treats that are non-toxic. The composition or components are coated onto the treat, incorporated into the treat, or both. Treats of the invention can be prepared by an extrusion or baking process similar to those used for dry food. Other processes also may be used to either coat the composition on the exterior of existing treat forms or inject the composition into an existing treat form.

In certain embodiments, the composition can be a toy. Toys include chewable toys such as artificial bones. The ginger can form a coating on the surface of the toy or on the surface of a component of the toy, be incorporated partially or fully throughout the toy, or both. There are a wide range of suitable toys currently marketed, for example, U.S. Pat. No. 5,339,771, U.S. Pat. No. 5,419,283, and references disclosed therein. This invention provides both partially consumable toys, for example, toys including plastic components, and fully consumable toys, for example, rawhides and various artificial bones. The invention preferably provides toys for use by a dog or a cat.

In certain embodiments, the companion animal is a senior animal.

In certain embodiments, the inflammatory disorder is arthritis.

In certain embodiments, the inflammatory condition is inflammatory bowel disorder.

In another embodiment, the invention encompasses methods for ameliorating or preventing a gastrointestinal disorder in a companion animal in need thereof comprising administering to the animal a pet food composition for a companion animal comprising an amount of protein, an amount of fat, an amount of carbohydrate, an amount of fiber and an effective amount of ginger to ameliorate or prevent the gastrointestinal disorder in the companion animal.

In certain embodiments, the companion animal is a dog.

In certain embodiments, the companion animal is a cat.

In certain embodiments, the gastrointestinal disorder is inflammatory bowel disorder.

In certain embodiments, the gastrointestinal disorder is diarrhea.

In certain embodiments, the invention encompasses dietary compositions for an animal including at least about 0.5% by weight ginger. In other embodiments, the ginger may be present at about 1% by weight. In other embodiments, the ginger may be present at about 1.5% by weight.

In various embodiments, the companion animals of the invention are the domestic cat (Felis domesticus) or the domestic dog (Canis domesticus). Other companion animals include, fish, bird and horse.

In various embodiments, the pet food composition can further include corn, poultry meal, palatability enhancer, potassium chloride, iodized salt, calcium carbonate, choline chloride, minerals, mineral premix, preservatives, vitamins, and mixtures thereof. In certain embodiments, the dietary food composition may further include L-tryptophan.

In certain embodiments, the dietary food composition can be administered to a senior animal or an animal with arthritis. In certain embodiments, the dietary composition can be administered to an animal with diarrhea or inflammatory bowel disorder.

In other embodiments, the invention encompasses methods for improving the quality of life of animals by feeding the animal a composition including at least about 0.5% by weight ginger. The compositions include ginger, for example, in amounts of about 1%, 1.5% or 2% by weight of the composition.

In another embodiment, the method encompasses feeding an animal in need thereof an amount of a composition including ginger, which is effective to enhance the animal's quality of life, wherein enhanced quality of life is evidenced by reduction in inflammatory disorders or diarrhea.

In another embodiment, the invention encompasses methods for treating gastrointestinal difficulties in an animal in need thereof including administering to the animal the dietary food composition of the invention. In certain embodiments, the animal may be a companion animal, such as a dog or a cat. In certain embodiments, the gastrointestinal difficulty may be inflammatory bowel disorder or diarrhea.

In another embodiment, the invention encompasses methods for treating inflammation in an animal in need thereof including administering to the animal a dietary food composition of the invention including ginger. In certain embodiments, the inflammation may be inflammatory bowel disorder or arthritis.

In another embodiment, the invention encompasses methods of screening for an agent that modulates gene expression comprising:
(a) preparing a first gene or gene family expression profile of a cell population comprising cells and/or assaying an activity of a protein encoded by at least one gene or a member of a gene family of Tables 10, 11, and 12 of a cell population;
(b) exposing the cell population to the agent;
(c) preparing a second gene or gene family expression profile of a ginger exposed cell population and/or assaying an activity of a protein encoded by at least one gene or a member of a gene family of Tables 10, 11, and 12 of the ginger exposed cell population; and
(d) comparing the first and second expression profiles or first and second activities to an expression profile and/or an activity of a cell population.

In another embodiment, the invention encompasses dietary food compositions including at least about 0.5% ginger by weight, wherein the dietary food compositions may alter expression of a gene. In certain embodiments, the gene may be selected from those listed herein. In certain embodiments, the gene may be any one included in Tables 10, 11, and 12 of the specification.

In another embodiment, the invention encompasses methods for identifying genes in an animal, which genes are modulated by a diet including ginger comprising administering a composition including ginger to the animal, obtaining a sample of nucleotides from the animal and comparing the number of nucleotides encoding a particular gene to a control animal. In certain embodiments, the gene may be any one included in Tables 10, 11, and 12 of the specification. In certain embodiments, the control animal may have the same disorder as the animal being tested or have no disorder.

In another embodiment, the invention encompasses methods of screening for an agent that modulates gene expression in a manner similar to ginger, comprising:
(a) preparing a first gene or gene family expression profile of a cell population comprising cells and/or assaying an activity of a protein encoded by at least one gene or a member of a gene family of Tables 10, 11, and 12 of a cell population;
(b) exposing the cell population to the agent;
(c) preparing a second gene or gene family expression profile of a ginger exposed cell population and/or assaying an activity of a protein encoded by at least one gene or a member of a gene family of Tables 10, 11, and 12 of the ginger exposed cell population; and
(d) comparing the first and second expression profiles or first and second activities to an expression profile and/or an activity of a cell population.

In another embodiment, the invention encompasses methods of monitoring the treatment of an animal with an inflammatory disorder, comprising:
(a) administering the dietary food to the animal;
(b) preparing a gene expression profile from a cell or tissue sample from the animal and/or assaying an activity of a protein encoded by at least one gene or a member of a gene family of Tables 10, 11, and 12; and
(c) comparing the animal expression profile or activity to an expression profile or activity obtained from a control.

In another embodiment, the invention encompasses detecting a change in an expression level of one or more genes or gene families associated with inflammation.

In certain embodiments, the activity of a protein encoded by a gene or member of a gene family may be assayed. In other embodiments, the assays may be conducted by themselves or in conjunction with determining an expression level. In other embodiments, the genes or member of gene families for which expression levels are determined may be the same or different as the genes encoding the proteins assayed. In certain embodiments, it may be desirable to determine the expression level of a gene and the activity level of the protein encoded by the gene. In other embodiments, it may be desirable to determine the expression level of one gene while determining the activity level of a protein encoded by another gene. Those skilled in the art will appreciate that the expression and/or activity level of any number of genes and proteins may be determined according to the present invention.

In another embodiment, the invention encompasses biomarker for determining whether an animal has an inflammatory disorder comprising obtaining a nucleotide or protein sample from the animal and comparing the expression of a gene selected from Tables 10, 11, and 12 to a control. In certain embodiments, two, three, four, five, six, seven, eight, nine, or ten genes may be compared. All of the genes listed in tables 10-12 may be compared.

The invention also encompasses biomarker for gastrointestinal inflammation or diarrhea including comparing the expression levels of CUBN, TLR, FOLR, FN1, TLN, and NUDT9. In certain embodiments, genes such as ACTA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, and GALT may be compared. Other genes such as TLR4, LITAF, HIST1H2BE, HLA-A, HLA-B, TAC3, CACB4, CDC25A, BPI, and TUBB2A may be compared.

The invention also encompasses methods of screening for an agent comparable to ginger that modulates the differentiation of genes, comprising: preparing a first gene or gene family expression profile and/or assaying for an activity of a protein encoded by a gene or member of a gene family in a cell population; contacting the cell population with ginger; preparing a second gene or gene family expression profile and/or assaying for an activity of a protein encoded by a gene or member of a gene family of the cell population after being contacted with the agent; and comparing the first and second expression profiles and/or activities.

In another embodiment, the invention encompasses methods of monitoring the treatment of an animal on a diet, which includes compositions including ginger comprising administering a diet, which includes compositions including ginger to the animal, preparing a gene or gene family expression profile and/or assaying for an activity of a protein encoded by a gene or member of a gene family from a cell or tissue sample from the animal and comparing the expression profile and/or activity to an expression profile and/or activity from a sample collected prior to treatment or earlier in the treatment program.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample is from a companion animal not having an arthritic condition, wherein said one or more biomarker is selected from Table 10, 11 or 12; and (b) identifying the companion animal corresponding to the test sample as being at risk of, or being predisposed to, developing an arthritic condition when expression of said one or more biomarker is found to be differentially expressed in the test sample compared to the control sample.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample is from a companion animal not having an arthritic condition, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the test sample as being at risk of, or being predisposed to, developing an arthritic condition when expression of said one or more biomarker is found to be differentially expressed in the test sample compared to the control sample.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample is a positive control sample comprising the average expression of such one or more biomarker in a plurality of reference samples that are derived from companion animals displaying symptoms of an arthritic condition, wherein said one or more biomarker is selected from Table 10, 11 or 12; and (b) identifying the companion animal corresponding to the said test sample as being at risk of, or being predisposed to, developing an arthritic condition when there is no statistically significant differential expression between said test sample and said positive control sample.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample is a positive control sample comprising the average expression of such one or more biomarker in a plurality of reference samples that are derived from companion animals displaying symptoms of an arthritic condition, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the said test sample as being at risk of, or being predisposed to, developing an arthritic condition when there is no statistically significant differential expression between said test sample and said positive control sample.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample comprises the average expression of such at least one or more biomarker in a plurality of reference samples that are derived from a companion animal displaying no symptoms of an arthritic condition wherein said one or more biomarker is selected from Table 10, 11 or 12; and (b) identifying the companion animal corresponding to the test sample as being at risk of, or being predisposed to, developing an arthritic condition when expression of said one or more biomarker is found to be differentially expressed in the test sample compared to the control sample.

In another embodiment, the invention encompasses a method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample comprises the average expression of such at least one or more biomarker in a plurality of reference samples that are derived from a companion animal displaying no symptoms of an arthritic condition wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF 139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the test sample as being at risk of, or being predisposed to, developing an arthritic condition when expression of said one or more biomarker is found to be differentially expressed in the test sample compared to the control sample.

In another embodiment, the invention encompasses a method of evaluating a companion animal to determine the companion animal's risk of, or predisposition to, developing an arthritic condition, the method comprising the steps of: (a) preparing a first sample for differential expression analysis from a test companion animal suspected of having a risk of, or predisposition to, developing an arthritic condition; (b) preparing a second sample for differential expression analysis from a companion animal having no symptoms of an arthritic condition; (c) hybridizing the first sample to a first array of hybridization probes to detect a first set of hybridization signals; (d) hybridizing the second sample to a second array of hybridization probes identical to the first array in step (c) to detect a second set of hybridization signals in an otherwise similar process to step (c); and (e) comparing the first and second sets of hybridization signals, wherein differential expression of one or more biomarker in companion animals associated with an arthritic condition identifies said test companion animal as having a risk of, or predisposition to, developing an arthritic condition, wherein said one or more biomarker is selected from Table 10, 11 or 12.

In another embodiment, the invention encompasses a method of evaluating a companion animal to determine the companion animal's risk of, or predisposition to, developing an arthritic condition, the method comprising the steps of: (a) preparing a first sample for differential expression analysis from a test companion animal suspected of having a risk of, or predisposition to, developing an arthritic condition; (b) preparing a second sample for differential expression analysis from a companion animal having no symptoms of an arthritic condition; (c) hybridizing the first sample to a first array of hybridization probes to detect a first set of hybridization signals; (d) hybridizing the second sample to a second array of hybridization probes identical to the first array in step (c) to detect a second set of hybridization signals in an otherwise similar process to step (c); and (e) comparing the first and second sets of hybridization signals, wherein differential expression of one or more biomarker in companion animals associated with an arthritic condition identifies said test companion animal as having a risk of, or predisposition to, developing an arthritic condition, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX).

In another embodiment, the invention encompasses a method of evaluating a companion animal to determine the companion animal's risk of, or predisposition to, developing an arthritic condition, the method comprising the steps of: (a) preparing a first sample for immunoassay analysis of one or more biomarker from a test companion animal suspected of having a risk of, or predisposition to, developing an arthritic condition; (b) preparing a second sample for immunoassay analysis of the same at least one or more biomarker from a companion animal having no symptoms of an arthritic condition; (c) comparing the immunoassay analysis of said first and second samples, wherein differential expression of one or more biomarker in said test companion animal identifies said test companion animal as having a risk of, or predisposition to, developing an arthritic condition, wherein said one or more biomarker is selected from Table 10, 11 or 12.

In another embodiment, the invention encompasses a method of evaluating a companion animal to determine the companion animal's risk of, or predisposition to, developing an arthritic condition, the method comprising the steps of: (a) preparing a first sample for immunoassay analysis of one or more biomarker from a test companion animal suspected of having a risk of, or predisposition to, developing an arthritic condition; (b) preparing a second sample for immunoassay analysis of the same at least one or more biomarker from a companion animal having no symptoms of an arthritic condition; (c) determining differential expression of said one or more biomarker in a companion animal corresponding to at least one biomarker associated with a risk of, or predisposition to, developing an arthritic condition in a companion animal between said first sample and said second sample; wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX).

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is from a companion animal not having a gastrointestinal inflammatory disorder, wherein said one or more biomarker is selected from Table 10, 11 or 12; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is from a companion animal not having a gastrointestinal inflammatory disorder, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is from a companion animal not having a gastrointestinal inflammatory disorder, wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is a positive control sample comprising the average expression of such one or more biomarker in a plurality of reference samples that are derived from companion animals displaying symptoms of an arthritic condition, wherein said one or more biomarker is selected from Table 10, 11 or 12; and (b) identifying the companion animal corresponding to the said test sample as being at risk of, or being predisposed to, developing a gastrointestinal inflammatory disorder when there is no statistically significant differential expression between said test sample and said positive control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is a positive control sample comprising the average expression of such one or more biomarker in a plurality of reference samples that are derived from companion animals displaying symptoms of a gastrointestinal inflammatory disorder, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARP), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the said test sample as being at risk of, or being predisposed to, developing a gastrointestinal inflammatory disorder when there is no statistically significant differential expression between said test sample and said positive control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is a positive control sample comprising the average expression of such one or more biomarker in a plurality of reference samples that are derived from companion animals displaying symptoms of a gastrointestinal inflammatory disorder, wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A; and (b) identifying the companion animal corresponding to the said test sample as being at risk of, or being predisposed to, developing a gastrointestinal inflammatory disorder when there is no statistically significant differential expression between said test sample and said positive control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample comprises the average expression of such at least one or more biomarker in a plurality of reference samples that are derived from a companion animal displaying no symptoms of an arthritic condition wherein said one or more biomarker is selected from Table 10, 11 or 12; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample comprises the average expression of such at least one or more biomarker in a plurality of reference samples that are derived from a companion animal displaying no symptoms of a gastrointestinal inflammatory disorder, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample comprises the average expression of such at least one or more biomarker in a plurality of reference samples that are derived from a companion animal displaying no symptoms of a gastrointestinal inflammatory disorder, wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

In another embodiment, the invention encompasses a method of evaluating a companion animal to determine the companion animal's risk of, or predisposition to, developing a gastrointestinal inflammatory disorder, the method comprising the steps of: (a) preparing a first sample for differential expression analysis from a test companion animal suspected of having a risk of, or predisposition to, developing a gastrointestinal inflammatory disorder; (b) preparing a second sample for differential expression analysis from a companion animal having no symptoms of a gastrointestinal inflammatory disorder; (c) hybridizing the first sample to a first array of hybridization probes to detect a first set of hybridization signals; (d) hybridizing the second sample to a second array of hybridization probes identical to the first array in step (c) to detect a second set of hybridization signals in an otherwise similar process to step (c); and (e) comparing the first and second sets of hybridization signals, wherein differential expression of one or more biomarker in companion animals associated with a gastrointestinal inflammatory disorder identifies said test companion animal as having a risk of, or predisposition to, developing a gastrointestinal inflammatory disorder, wherein said one or more biomarker is selected from Table 10, 11 or 12.

In another embodiment, the invention encompasses a method of evaluating a companion animal to determine the companion animal's risk of, or predisposition to, developing a gastrointestinal inflammatory disorder, the method comprising the steps of: (a) preparing a first sample for differential expression analysis from a test companion animal suspected of having a risk of, or predisposition to, developing a gastrointestinal inflammatory disorder; (b) preparing a second sample for differential expression analysis from a companion animal having no symptoms of a gastrointestinal inflammatory disorder; (c) hybridizing the first sample to a first array of hybridization probes to detect a first set of hybridization signals; (d) hybridizing the second sample to a second array of hybridization probes identical to the first array in step (c) to detect a second set of hybridization signals in an otherwise similar process to step (c); and (e) comparing the first and second sets of hybridization signals, wherein differential expression of one or more biomarker in companion animals associated with a gastrointestinal inflammatory disorder identifies said test companion animal as having a risk of, or predisposition to, developing a gastrointestinal inflammatory disorder, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX).

In another embodiment, the invention encompasses a method of evaluating a companion animal to determine the companion animal's risk of, or predisposition to, developing a gastrointestinal inflammatory disorder, the method comprising the steps of: (a) preparing a first sample for differential expression analysis from a test companion animal suspected of having a risk of, or predisposition to, developing a gastrointestinal inflammatory disorder; (b) preparing a second sample for differential expression analysis from a companion animal having no symptoms of a gastrointestinal inflammatory disorder; (c) hybridizing the first sample to a first array of hybridization probes to detect a first set of hybridization signals; (d) hybridizing the second sample to a second array of hybridization probes identical to the first array in step (c) to detect a second set of hybridization signals in an otherwise similar process to step (c); and (e) comparing the first and second sets of hybridization signals, wherein differential expression of one or more biomarker in companion animals associated with a gastrointestinal inflammatory disorder identifies said test companion animal as having a risk of, or predisposition to, developing a gastrointestinal inflammatory disorder, wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A.

In another embodiment, the invention encompasses a method of evaluating a companion animal to determine the companion animal's risk of, or predisposition to, developing a gastrointestinal inflammatory disorder, the method comprising the steps of: (a) preparing a first sample for immunoassay analysis of one or more biomarker from a test companion animal suspected of having a risk of, or predisposition to, developing a gastrointestinal inflammatory disorder; (b) preparing a second sample for immunoassay analysis of the same at least one or more biomarker from a companion animal having no symptoms of a gastrointestinal inflammatory disorder; (c) comparing the immunoassay analysis of said first and second samples, wherein differential expression of one or more biomarker in said test companion animal identifies said test companion animal as having a risk of, or predisposition to, developing a gastrointestinal inflammatory disorder, wherein said one or more biomarker is selected from Table 10, 11 or 12.

In another embodiment, the invention encompasses a method of evaluating a companion animal to determine the companion animal's risk of, or predisposition to, developing a gastrointestinal inflammatory disorder, the method comprising the steps of: (a) preparing a first sample for immunoassay analysis of one or more biomarker from a test companion animal suspected of having a risk of, or predisposition to, developing a gastrointestinal inflammatory disorder; (b) preparing a second sample for immunoassay analysis of the same at least one or more biomarker from a companion animal having no symptoms of a gastrointestinal inflammatory disorder; (c) determining differential expression of said one or more biomarker in a companion animal corresponding to at least one biomarker associated with a risk of, or predisposition to, developing a gastrointestinal inflammatory disorder in a companion animal between said first sample and said second sample; wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX).

In another embodiment, the invention encompasses a method of evaluating a companion animal to determine the companion animal's risk of, or predisposition to, developing a gastrointestinal inflammatory disorder, the method comprising the steps of: (a) preparing a first sample for immunoassay analysis of one or more biomarker from a test companion animal suspected of having a risk of, or predisposition to, developing a gastrointestinal inflammatory disorder; (b) preparing a second sample for immunoassay analysis of the same at least one or more biomarker from a companion animal having no symptoms of a gastrointestinal inflammatory disorder; (c) determining differential expression of said one or more biomarker in a companion animal corresponding to at least one biomarker associated with a risk of, or predisposition to, developing a gastrointestinal inflammatory disorder in a companion animal between said first sample and said second sample; wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A.

In another embodiment, the invention encompasses a kit for the diagnosis, prognosis or monitoring an arthritic condition in a companion animal, comprising at least one or more agent capable of detecting at least one or more biomarker selected from Table 10, 11 or 12; and instructions for using such at least one or more agent to evaluate the risk, predisposition or presence of a process leading to an arthritic condition in such companion animal.

In another embodiment, the invention encompasses a kit for the diagnosis, prognosis or monitoring an arthritic condition in a companion animal, comprising at least one or more agent capable of detecting at least one or more biomarker selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); and instructions for using such at least one or more agent to evaluate the risk, predisposition or presence of a process leading to an arthritic condition in such companion animal.

In another embodiment, the invention encompasses a method of identifying an ingredient of a pet food composition suitable to prevent, ameliorate the symptoms of, or treat, an arthritic condition in a companion animal, said method comprising the steps of: (a) contacting a first population of cells of said companion animal capable of expressing one or more biomarker disclosed in Table 10, 11 or 12 with a test ingredient; (b) collecting a first sample from said first population of cells, said sample containing a quantity of said one or more biomarker; (c) determining the amount of said one or more biomarker in said first sample; (d) comparing the amount of said one or more biomarker is said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, an arthritic condition in a companion animal.

In another embodiment, the invention encompasses a method of identifying an ingredient of a pet food composition suitable to prevent, ameliorate the symptoms of, or treat, an arthritic condition in a companion animal, said method comprising the steps of: (a) contacting a first population of cells of said companion animal capable of expressing one or more biomarker selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); (b) collecting a first sample from said first population of cells, said sample containing a quantity of said one or more biomarker; (c) determining the amount of said one or more biomarker in said first sample; (d) comparing the amount of said one or more biomarker is said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, an arthritic condition in a companion animal.

In another embodiment, the invention encompasses a method of identifying an ingredient of a pet food composition suitable to prevent, ameliorate the symptoms of, or treat, a gastrointestinal inflammatory disorder in a companion animal, said method comprising the steps of: (a) contacting a first population of cells of said companion animal capable of expressing one or more biomarker disclosed in Table 10, 11 or 12 with a test ingredient; (b) collecting a first sample from said first population of cells, said sample containing a quantity of said one or more biomarker; (c) determining the amount of said one or more biomarker in said first sample; (d) comparing the amount of said one or more biomarker in said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, a gastrointestinal inflammatory disorder in a companion animal.

In another embodiment, the invention encompasses a method of identifying an ingredient of a pet food composition suitable to prevent, ameliorate the symptoms of, or treat, a gastrointestinal inflammatory disorder in a companion animal, said method comprising the steps of (a) contacting a first population of cells of said companion animal capable of expressing one or more biomarker selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), or WW domain-containing oxidoreductase (WWOX); (b) collecting a first sample from said first population of cells, said sample containing a quantity of said one or more biomarker; (c) determining the amount of said one or more biomarker in said first sample; (d) comparing the amount of said one or more biomarker is said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, a gastrointestinal inflammatory disorder in a companion animal.

In another embodiment, the invention encompasses a method of identifying a ingredient of a pet food composition suitable to prevent, ameliorate the symptoms of, or treat, an arthritic condition in a companion animal, said method comprising the steps of (a) contacting a first population of cells of said companion animal capable of expressing one or more biomarker selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A; (b) collecting a first sample from said first population of cells, said sample containing a quantity of said one or more biomarker; (c) determining the amount of said one or more biomarker in said first sample; (d) comparing the amount of said one or more biomarker is said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, an arthritic condition in a companion animal.

In other embodiments, the invention encompasses a composition comprising at least two oligonucleotides, wherein each of the oligonucleotides comprises a sequence that specifically hybridizes to one or more genes or members of the gene families disclosed herein. In some aspects, the composition may comprise at least 1, 2, 3, 4, 5, 6, 7, 8, 9, 10 oligonucleotides, wherein each of the oligonucleotides comprises a sequence that specifically hybridizes to one or more genes or members of the gene families disclosed herein. In some embodiments, one or more of the oligonucleotides may be attached to a solid support. The solid support may be any known to those skilled in the art including, but not limited to, a membrane, a glass support, a filter, a tissue culture dish, a polymeric material or a silicon support.

In other embodiments, the invention encompasses a solid support to which is attached at least two oligonucleotides, wherein each of the oligonucleotides comprises a sequence that specifically hybridizes to at least one or more genes or members of the gene families disclosed herein. In some embodiments, at least one oligonucleotide is attached covalently to the solid support. In some embodiments, at least one oligonucleotide is attached non-covalently to the solid support. Oligonucleotides may be attached to the solid supports of the invention at any density known to those skilled in the art, for example, at about at least 10 different oligonucleotides in discrete locations per square centimeter, at about at least 100 different oligonucleotides in discrete locations per square centimeter, at about at least 1000 different oligonucleotides in discrete locations per square centimeter and/or at about at least 10,000 different oligonucleotides in discrete locations per square centimeter. The selection of an appropriate density for a given application is a routine procedure for those skilled in the art.

The invention also encompasses computer systems comprising a database containing information identifying the expression level of one or more members of one or more genes disclosed herein and/or the activity level of one or more proteins encoded by a genes or member of the gene families disclosed herein and a user interface to view the information. In certain embodiments, the database may further comprise sequence information for one or more genes or members of the gene families disclosed herein. In other embodiments, the database may comprise information identifying the expression level for one or more genes or one or more members of one or more of the gene families in the set of gene families expressed in response to a ginger diet. The database may further contain or be linked to descriptive information from an external database, which information correlates said genes and/or gene families to records in the external database.

The invention also encompasses methods of using the disclosed computer systems to present information identifying the expression level in a tissue or cell of a set of genes and/or gene families comprising at least one or more genes or gene families disclosed herein, comprising comparing the expression level of at least one or more genes or gene families disclosed herein in the tissue or cell to the level of expression of the gene in the database. The invention also includes methods of using the disclosed computer systems to present information identifying the activity level in a tissue or cell of one or more proteins encoded by one or more genes and/or members of a gene family comprising at least one of the genes or gene families as disclosed herein, comprising comparing the activity level of at least one protein encoded by one gene or member of a gene family disclosed herein in the tissue or cell to the level of activity of the protein in the database.

The invention encompasses pet food compositions that provide beneficial effects and the methods of preparing and use thereof. The beneficial effects may be anti-inflammatory. The beneficial effects may be anti-arthritic. The beneficial effects may be anti-diarrheal. The present invention provides methods for improving or enhancing the response of an animal to inflammatory bowel disease or diarrhea.

The present invention provides use of ginger to attenuate the symptoms associated with gastrointestinal inflammation and diarrhea. The invention encompasses methods for modifying key inflammatory genes and proteins to improve oxidative stress, DNA repair, protein damage, nutrient absorption and immune health comprising administering ginger or ginger containing foods to an animal. The present invention provides biomarker of therapeutic intervention with ginger such as CUBN, TLR, FOLR, FN1, TLN, and NUDT9 etc. The present invention provides methods of modifying gene expression to improved symptoms of diarrhea in cats with inflammatory bowel disease and improved considerably in both healthy and chronic diarrhea animals.

The present invention provides methods for modifying gene expression in an animal comprising administering to the animal a diet comprising ginger. Ginger may be present in the diet at 0.5%, 1%, 2%, 3%, 4%, 5%, 6%, 7%, 8%, 9%, or 10% by weight. The ginger may affect genes involved in apoptosis, folate transport, vitamin transport, inflammation, cell integrity, DNA repair, calcium binding and storage, vitamin binding, collagen formation, cytoskeletal structure and function, integrin signaling, and cholesterol and fatty acid synthesis.

The present inventors have identified the effects of ginger to reduce inflammatory markers and improve markers of cartilage synthesis in cats and dogs with arthritis. Feeding a food containing 0.5% ginger reduced IL-1 beta and IL-6, and tended to lower IFN-gamma markers associated with inflammation/arthritis. In addition, ginger stimulated cartilage synthesis as indicated by a marker (CPII) of collagen synthesis and improved the ratio of collagen synthesis to degradation (CPII:CTXII) with a greater percent increase observed in arthritic cats (57%) compared to non-arthritic (8%). In dogs, feeding a control food +1% ginger resulted in improvements in markers of bone formation, bone turnover, and BAP:NTX ratio. This suggests that dogs fed ginger had improved balance between synthesis and turnover of bone that is normally upset in arthritic animals.

Ginger may be in the form of the ground powder, freshly ground, spray dried or freeze-dried, wet root, extract, or oils, can be incorporated in foods or drinks.

Ginger may be incorporated into a food or used as a nutritional supplement in many different forms including ginger extracts such as ginger powder extracts, ginger fluid extracts, ginger powder, and one or more active compounds of ginger; parts of or whole ginger plants; tinctures thereof; and mixtures thereof. In addition, for any specific active compound of ginger for which suitable synthesis routes are known, the active compound can be prepared synthetically. Preferably, the second ingredient of the nutritional supplement of the present invention is selected from ginger extract, and ginger root powder.

Extracts of ginger can be prepared from fresh or dried ginger root which is prepared by extraction with methanol, ethanol, isopropanol, acetone, ethyl acetate, carbon dioxide, hexane, methylene chloride, chloroform or other solvents or solvent mixtures of comparable polarity.

Ginger contains 1-4% essential oil (oleoresin). Many chemical investigations have been carried out on the constituents of the essential oil of ginger. All together more than 200 different volatile compounds have been identified in the essential oil of ginger. The essential oil of ginger contains a mixture of various terpenes as well as some other non-terpenoid compounds.

The active compounds of ginger which may be employed in the present invention include, but are not limited to, 1,8-cineole, 10-dehydrogingerdione, 10-gingerol, 6-gingerdione, 6-gingerol, 6-shogaol, 8-β-17-epoxy-λ-trans-12-ene-15,16-diol, 8-gingerol, 8-shogaol, 9-oxo-nerolidol, acetaldehyde, acetic acid, alanine, α-linolenic-acid, α-phellandrene, α-piene, α-terpinene, α-terpineol, α-zingiberene, ar-curcumene, arginine, ascorbic acid, asparagine, β-bisabolol, β-carotene, β-elemene, O-eudesmol, β-ionone, β-myrcene, β-phellandrene, β-pinene, β-selinene, β-sesquiphellandrene, β-sitosterol, β-thujone, bornyl-acetate, boron, caffeic acid, calcium, camphene, camphor, capric acid, caprylic acid, capsaicin, caryophyllene, chavicol, chlorogenic acid, chromium, citral, citronellal, citronellal, cobalt, copper, cumene, curcumin, cystine, delphinidin, Δ-cadinene, elemol, ethyl acetate, ethyl-myristate, farnesal, farnesene, ferulic acid, furfural, γ-aminobutyric acid, γ-terpineine, geranial, geraniol, geranyl-acetate, gingerenone, glutamic acid, glycine, hexahydrocurcumin, histidine, isogingerenone-B, isoleucine kaempferol, lecithin, limonene, linoleic acid, magnesium, manganese, methionine, mufa, myrecene, myricetin, myristic acid, neral, nerol, nerolidol, niacin, nickel, oleic acid, oxalic acid, p-coumaric acid, p-cymene, p-hydroxy-benzoic acid, palmitic acid, pantothenic acid, paradol, patchoulic alcohol, phenylalanine, quercetin, riboflavin, selenium, shikimic-acid, terpinen-4-ol, thiamin, tryptophan, vanillic acid, vanillin, zinc, and zingerone. In addition, mixtures of two or more of these active compounds may be employed.

Without being bound by theory, the benefits of the invention may be the result of physiological effects from the addition of ginger or derivatives thereof to an animal's diet. Similarly, the antioxidants, choline, and other nutrients may play a role in enhancing a senior or super senior animal's quality of life.

The invention encompasses food compositions or supplements wherein ginger is present in an effective amount to treat, manage, or ameliorate a condition in an animal. The effective amount of ginger may vary depending on such factors as the patient being treated, the particular mode of administration, the activity of the particular active ingredients employed, the age, bodyweight, general health, sex and diet of the patient, time of administration, rate of excretion, the particular combination of ingredients employed, the total content of the main ingredient of the nutritional supplement or nutritionally complete diet, and the severity of the illness or symptom. It is within the skill of the person of ordinary skill in the art to account for these factors.

A “nutritionally complete diet” is a diet that includes sufficient nutrients for maintenance of normal health of a healthy animal on the diet. The methods of this invention utilize compositions that are not intended to be restricted by any specific listing of proteinaceous or fat ingredients or product form. The compositions can be prepared in, for example, a dry, canned, wet, or intermediate moisture form using conventional pet food processes. In some embodiments, the moisture content is from about 10% to about 90% of the total weight of the composition. In other embodiments, the moisture content is from about 65% to about 75% of the total weight of the composition.

The food composition may further contain other ingredients such as corn, poultry meal, grease, palatability enhancers, potassium chloride, iodized salt, calcium carbonate, choline chloride, mineral premix, preservative, vitamin premix. The food may contain protein. The protein may be present in about 5 to 50% by weight. The protein may be animal protein. Animal protein may be part of the total protein. Animal protein may be 50%, 70%, 80%, 90,%, 95%, 99% or 100% of the total protein. The food may contain antioxidants, such as vitamin E. Antioxidant may be present in between about 0.0001 U/g and 3.0 U/g food, such as, e.g., 0.18 U/g. The food may contain eicosapentaenoic acid (EPA) at between 1% and 5% by weight. Fatty acids may also be included such as n-3 and n-6 fatty acids. Fatty acids may be present in about 0.05% to 5% by weight. n-3 fatty acids may be present in about 0.0001% to 2%. n-6 fatty acids may be present in about 0.5% to 5% by weight. The food may contain fiber, such as crude fiber. Fiber may be present in between 0.001% and 10% by weight.

The effective amount of the nutritional supplement will vary depending on such factors as the patient being treated, the particular mode of administration, the activity of the particular active ingredients employed, the age, bodyweight, general health, sex and diet of the patient, time of administration, rate of excretion, the particular combination of ingredients employed, the total content of the main ingredient of the nutritional supplement, and the severity of the illness or symptom. It is within the skill of the person of ordinary skill in the art to account for these factors.

The ginger dietary foods or supplements of the present invention may be formulated using a safe and effective amount of ginger as discussed herein to provide one or more of the beneficial effects of the invention described herein, and one or more of the optional ingredients which may be obtained from slippery elm or green tea, as well as one or more of the additional optional ingredients described below. The nutritional supplement of the present invention may also be formulated with a pharmaceutically acceptable carrier.

The nutritional supplement of the present invention may be formulated in any orally acceptable dosage form including, but not limited to, capsules, tablets, lozenges, troches, hard candies, powders, sprays, gels, elixirs, syrups, and suspensions or solutions.

An acceptable carrier may include, but is not limited to: (a) carbohydrates including sweeteners, more preferably, fructose, sucrose, sugar, dextrose, starch, lactose, maltose, maltodextrins, corn syrup solids, honey solids, commercial tablet nutritional supplements including Emdex®, Mor-Rex®, Royal-T®, Di-Pac®, Sugar-Tab®, Sweet-Rex®, New-Tab®, (b) sugar alcohols including mannitol, sorbitol, xylitol, and (c) various relatively insoluble excipients including dicalcium phosphate, calcium sulfate, calcium carbonate, microcrystalline cellulose and other pharmaceutical tableting ingredients.

Alternatively, the ginger foods or supplements of the present invention may be formulated in liquid form, such as syrups, mouthwashes or sprays with a solvent or dispersant such as water, or other liquids in a pharmaceutically acceptable carrier for repeated delivery of the nutritional supplement to oral and oropharyngeal mucous membranes over a sustained period of time. Preferably, the treatment time is about 5 to 60 minutes, and more preferably about 20 to 30 minutes, so as to permit a prolonged contact of the nutritional supplement with mouth and throat tissues. Alternatively, such formulations can be in a form suitable for dilution with water or other materials prior to use.

The ginger foods or supplements of the present invention may also be formulated in chewable nutritional supplements such as soft candy, gum drops, liquid filled candies, chewing gum bases and dental supplies, such as toothpastes and mouthwashes by further including fructose, sucrose, or saccharin in the nutritional supplement, as needed. In use, the chewable composition is retained in the mouth over a sustained period of time of preferably about 5 to 60 minutes, and more preferably about 20 to 30 minutes.

The ginger foods or supplements of the present invention may be formulated in capsule form with or without diluents. For capsules, useful diluents include lactose and dried corn starch. When suspensions are employed, emulsifying and/or suspending agents may be employed in the suspensions. In addition, solid compositions including one or more of the ingredients of the lozenges described above may be employed in soft and hard gelatin capsules.

The ginger foods or supplements of the present invention may be formulated into a nasal aerosol or inhalant composition. Such a composition may be prepared using well-known techniques. For these types of formulations, suitable carriers may include the following ingredients: saline with one or more preservatives, absorption promoters to enhance bioavailability, fluorocarbons and/or other conventional solubilizing or dispersion agents.

Other materials, which may optionally be included in the nutritional supplements of the present invention, include inositol, other B-complex vitamins, and anti-inflammatories. Also, ingredients such as sweeteners, flavorants, coloring agents, dyes, preservatives, emulsifying agents, suspending agents, melting agents, excipients, and solvents or diluents such as water, ethanol, propylene glycol, glycerin and various combinations thereof, may be included in the ginger foods or supplements of the present invention.

The optional sweeteners, which may be used in the ginger foods or supplements of the present invention include, but are not limited to, saccharin, aspartame, cyclamates, acesulfame K, neohesperidin dihydrochalcone, other sweeteners, and mixtures thereof, which may be added to the carrier in amounts sufficiently low so as not to chemically interact with the main ingredients of the nutritional supplement.

The optional flavorants which may be used in the ginger foods or supplements of the present invention include, but are not limited to, peppermint, peppermint-menthol, eucalyptol wintergreen, licorice, clove, cinnamon, spearmint, cherry, lemon, orange lime, menthol and various combinations thereof.

Although the methods of the invention may improve an animal's quality of life by enhancing all of the above described characteristics or improving all of the described biological pathways, it is not necessary to demonstrate substantial improvements in each of the characteristics or pathways to achieve the “enhanced quality of life” as defined herein.

When the compositions are administered to an animal, the animal experiences an enhanced quality of life, e.g., decreased inflammatory responses, increased collagen synthesis, increased cartilage synthesis, reduced diarrhea, reduced arthritis, decreased cartilage degradation, DNA damage and repair pathways, glycogen synthesis and degradation, glycolysis, gluconeogenesis, the pentose phosphate pathway and the electron transport pathway as indicated by overall beneficial changes at the cellular level. Methods for determining these measurements of quality of life are known to skilled artisans. Potential markers of antioxidant status may include serum vitamin E, ORAC, glutathione peroxidase, alkanels, and/or cell damage indicators. Further, vitality can be measured by various means, including an analysis of metabolism and antioxidant markers, as well as through clinical studies with follow-up questions to participating pet owners. Similarly, cartilage protection can be measured by various means, including an analysis of arthritis biomarker. Potential arthritis biomarker may include type II collagen synthesis, matrix metalloproteinase, osteocalcin, alkaline phosphatase activity, COMP, and fragments of cartilage damage. Muscle mass maintenance can be measured by various means, including an analysis of body composition, and digestibility can be measured by various means, including clinical studies with follow-up questions to participating pet owners and animal feeding to determine the percentage of nutrients digested. Skin and pelage quality can be measured by various means, including clinical studies with follow-up questions to participating pet owners.

The methods of the invention are useful for enhancing the quality of life of animals, including primates (e.g., monkeys, chimpanzees, etc.), companion animals (e.g., dogs, cats, horses, etc.), farm animals (e.g., goats, sheep, swine, cattle, etc.), laboratory animals (e.g., mice, rats, etc.), birds (e.g., domestic birds such as canaries, parrots, etc. and commercial birds such as chickens, ducks, turkeys, etc.), rodents (e.g., hamsters, guinea pigs, gerbils, rabbits, hedgehogs, ferrets, chinchillas, etc.), and wild, exotic, and zoo animals (e.g., wolves, bears, deer, etc.). In various embodiments, the animal is a cat, a dog, or a horse.

The food compositions comprise ginger. Ginger may be present at about 0.5%, about 1%, about 1.5%, about 2%, about 3%, about 4%, about 5%, about 6%, about 7%, about 8%, about 9%, or about 10% by weight. The food composition may comprise other ingredients. The other ingredients may be selected from water, protein, fiber, carbohydrate, fat, soluble fiber, insoluble fiber, docosahexaenoic acid (DHA), eicosapentaenoic acid (EPA), omega-3 fatty acids, omega-6 fatty acids, tocopherols, ascorbate, calcium, chloride, cysteine, magnesium, manganese, methionine, phosphorous, potassium, iron, and essential amino acids.

When the composition is an animal food, vitamins and minerals preferably are included in amounts required to avoid deficiency and maintain health. These amounts are readily available in the art. The National Research Council (NRC), for example, provides recommended amounts of such ingredients for farm animals. See, e.g., Nutrient Requirements of Swine (10th Rev. Ed., Nat'l Academy Press, Wash. D.C., 197298), Nutrient Requirements of Poultry (9th Rev. Ed., Nat'l Academy Press, Wash. D.C., 1994), Nutrient Requirements of Horses (Fifth Rev. Ed., Nat'l Academy Press, Wash. D.C., 1989), Nutrient Requirements of Dogs and Cats (Nat'l Academy Press, Wash. D.C., 2006). The Association of American Feed Control Officials Inc. (AAFCO), for example, provides recommended amounts of such ingredients for dogs and cats. See Association of American Feed Control Officials Inc. Official Publication (2009). Examples of vitamins useful as food additives include vitamin A, B1, B2, B6, B12, C, D, E, K, H (biotin), K, folic acid, inositol, niacin, and pantothenic acid. Examples of minerals and trace elements useful as food additives include calcium, phosphorus, sodium, potassium, magnesium, copper, zinc, chloride, and iron salts.

The methods of the present invention include compositions that may further contain other additives known in the art. Such additives are present in amounts that do not impair the purpose and effect provided by the invention. Examples of additives include, for example, substances with a stabilizing effect, processing aids, substances that enhance palatability, coloring substances, and substances that provide nutritional benefits.

Stabilizing substances include, for example, substances that tend to increase the shelf life of the composition. Potentially suitable examples of such substances include, for example, preservatives, antioxidants, synergists and sequesterants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

Additives for coloring, palatability and nutritional purposes include, for example, colorants (e.g., iron oxide, such as the red, yellow, or brown forms); sodium chloride, potassium citrate, potassium chloride, and other edible salts; vitamins; minerals; and flavoring. Such additives are known in the art. See, e.g., U.S. Pat. No. 3,202,514. See also, U.S. Pat. No. 4,997,671. Flavorants include, for example, dairy product flavorants (e.g., milk or cheese), meat flavorants (e.g., bacon, liver, beef, poultry, or fish), oleoresin, pinacol, and the various flavorants identified in the trade by a FEMA (Flavor Extract Manufacturers Association) number. Flavorants help provide additional palatability, and are known in the art. See, e.g., U.S. Pat. No. 4,997,672. See also, U.S. Pat. No. 5,004,624, U.S. Pat. No. 5,114,704, U.S. Pat. No. 5,532,010, and U.S. Pat. No. 6,379,727. The concentration of such additives in the composition typically may be up to about 5% by weight. In some embodiments, the concentration of such additives (particularly where such additives are primarily nutritional balancing agents, such as vitamins and minerals) is from about 0% to about 2.0% by weight. In some embodiments, the concentration of such additives (again, particularly where such additives are primarily nutritional balancing agents) is from about 0% to about 1.0% by weight.

Supplements include, for example, a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the Association of American Feed Control Officials Inc. Official Publication, p. 220 (2003). Supplements may be in various forms including, for example, powders, liquids, syrups, pills, encapsulated compositions, and the like.

Treats include, for example, compositions that are given to an animal to entice the animal to eat during a non-meal time. Treats for canines include, for example, dog bones. Treats may be nutritional, wherein the composition comprises one or more nutrients, and may, for example, have a composition as described above for food. Non-nutritional treats encompass any other treats that are non-toxic.

The invention may further provide ginger in a pet toy, wherein the toy is likely to enter the pet's oral cavity. Toys include, for example, chewable toys. Toys for dogs include, for example, artificial bones. There is a wide range of suitable toys currently marketed. See, e.g., U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). The invention provides both partially consumable toys (e.g., toys comprising plastic components) and fully consumable toys (e.g., rawhides and various artificial bones). It should be further recognized that this invention provides toys for both human and non-human use, particularly for companion, farm, and zoo animal use, and particularly for dog, cat, or bird use.

In preparing a composition for use with the methods of the present invention, any ingredient (e.g., fish oil) generally may, for example, be incorporated into the composition during the processing of the formulation, such as during and/or after mixing of other components of the composition. Distribution of these components into the composition can be accomplished by conventional means. In one embodiment, ground animal and poultry proteinaceous tissues are mixed with the other ingredients, including fish oils, cereal grains, other nutritionally balancing ingredients, special-purpose additives (e.g., vitamin and mineral mixtures, inorganic salts, cellulose and beet pulp, bulking agents, and the like); and water that is sufficient for processing is also added.

Methods of the present invention include utilizing compositions that can be prepared in a dry form using conventional processes. In one embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing.

The compositions may also be designed to be easier to chew. Canine and feline foods are typically formulated based on life stage (age), size, body composition, and breed. In the methods of this invention, some embodiments address specific nutritional differences between senior regular or small breed dogs, large breed dogs, and cats.

All percentages expressed herein are on a weight by dry matter basis unless specifically stated otherwise.

The present invention is directed, in part, to a method for enhancing the quality of life of an animal. The method may comprise feeding a senior animal a composition in an amount effective to alleviate inflammation, such as inflammation associated with arthritis.

### Gene Regulation

It is contemplated herein that modulating the expression levels of the genes disclosed herein may have therapeutic value with regard to the treatment of diseases or disorders associated with the various biological pathways. Such determination may be made on a gene by gene basis without undue experimentation, for example, by assessing expression levels in tissues as well as in blood samples, or by assaying expression levels in vitro in cells or cell lines relevant to particular disease states and suitable for such experimentation. In vivo models of disease might also be utilized in such experimentation. The nature of these and other suitable additional assays would be familiar to one of skill in the art. Thus, based on the genomic data disclosed herein, the invention also relates to methods to enhance the quality of life of an animal by modulating the expression level of one or more genes listed on Tables 10-12 (i.e., up or down regulation as indicated therein) in an animal in order to mimic the pattern of expression seen in vivo after administration of the pet food compositions of the present invention.

Modulation of gene expression levels may be achieved using known modulators of gene expression suitable for administration in vivo, including, but not limited to, ribozymes, antisense oligonucleotides, triple helix DNA, RNA aptamers and/or double stranded RNA directed to an appropriate nucleotide sequence of a gene of interest. These inhibitory molecules may be created using conventional techniques by one of skill in the art without undue burden or experimentation. For example, modification (e.g., inhibition) of gene expression may be obtained by designing antisense molecules, DNA or RNA, to the control regions of the genes discussed herein, i.e., to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Can, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to modulate gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional RNAi technologies. Numerous references describing such technologies exist and include, for example, WO 99/32619; Miller et al. Cell Mol Neurobiol 25:1195-207 (2005); Lu et al. Adv Genet 54:117-42 (2005).

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes discussed herein. Such DNA sequences may be incorporated into a wide variety of vectors with suitable RNA polymerase promoters such as T7 or SP6 according to conventional methods. Alternatively, cDNA constructs that synthesize antisense RNA constitutively or inducibly can be introduced into cell lines, cells, or tissues using methods familiar to one of skill in the art. Vectors may be introduced into cells or tissues by many available means, and may be used in vivo, in vitro or ex vivo. For ex vivo therapy, vectors may be introduced into stem cells taken from an animal and clonally propagated for autologous transplant back into that same animal. Delivery by transfection and by liposome injections may be achieved using methods that are well known in the art.

The instant invention also includes a method to identify an animal that might benefit from feeding a composition as disclosed herein comprising measuring the gene expression levels of any one or more genes listed in Tables 10-12 in said animal and comparing said levels to the gene expression levels seen in Tables 10-12 wherein an animal with levels different than those seen in Tables 10-12 (e.g., up regulated versus down regulated) would be identified as potentially benefiting from feeding a composition of the present invention.

Additionally, as discussed above, improvements in quality of life is also reflected at the genomic level, as evidenced by gene chip data which indicate beneficial changes on the expression of a majority of genes associated with various important biological pathways including inflammatory responses, collagen synthesis, vitamin transport, cartilage synthesis, cartilage degradation, apoptosis, and DNA damage and repair pathways. The identities of these genes are provided in the Examples below.

It is reported herein the surprising discovery that the enhanced quality of life of an animal achieved by administration of the compositions of the present invention is reflected at the genomic level. While it may be that a change in expression of any one gene disclosed in the tables presented below may result in beneficial or deleterious biological effects, the data presented herein indicate that, overall, the observed expression profiles are consistent with the beneficial biological effects seen in vivo after administration of the diets disclosed herein. Specifically, gene chip data indicate that the expression of genes that encode proteins associated with or related to several biological pathways such as collagen synthesis, apoptosis, vitamin transport, inflammatory responses, vitamin binding, calcium binding and storage, cholesterol and fatty acid biosynthesis, kidney cancer, and DNA damage and repair pathways are, for the most part, beneficially altered through administration to the animal of compositions described herein. Thus, the invention also relates to methods of measuring or characterizing the enhancement in the quality of life of an animal, particularly a senior animal, fed a composition described herein by quantitating the gene expression levels of one or more genes selected from a group consisting of those disclosed in the tables presented below in said animal and comparing said levels in the animal to levels in the animal prior to administration of the feed composition. Quantitation of gene expression may be carried out in numerous ways familiar to one of skill in the art and include such techniques as RT-PCR as well as gene chip assays and northern blotting. Thus, it is contemplated herein that the expression levels detected may be used, for example, in methods to measure enhancement in the quality of life of an animal as disclosed herein.

### Assay Formats

The genes and sequences identified as being differentially expressed in the cell population induced to differentiate as well as related sequences may be used in a variety of nucleic acid detection assays to detect or quantitate the expression level of a gene or multiple genes in a given sample. For example, traditional northern blotting, nuclease protection, RT-PCR, QPCR (quantitative RT-PCR), Taqman® and differential display methods may be used for detecting gene expression levels. Those methods are useful for some embodiments of the invention. However, methods and assays of the invention are most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, silicon or glass based chips, etc. Such supports and hybridization methods are widely available, for example, those disclosed by WO 95/11755. Any solid surface to which oligonucleotides can be bound, directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be any convenient size and may preferably be on the order of a square centimeter.

Oligonucleotide probe arrays for expression monitoring can be made and used according to any techniques known in the art (see for example, Lockhart et al., (1996) Nat. Biotech. 14, 1675-1680; McGall et al., (1996) Proc. Nat. Acad. Sci. USA 93, 13555-13460). For instance, such arrays may also contain oligonucleotides that are complementary or hybridize to at least about 1, 2, 3, 4, 5, 6, 7, 8, 9, 10, 20, 30, 50, 70 or more the genes described herein.

The genes which are assayed according to the present invention are typically in the form of mRNA or reverse transcribed mRNA. The genes may be cloned or not. The genes may be amplified or not. The cloning itself does not appear to bias the representation of genes within a population. However, it may be preferable to use polyadenylated RNA as a source, as it can be used with less processing steps.

Probes based on the sequences of the genes described above may be prepared by any commonly available method. Oligonucleotide probes for interrogating the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40 or 50 nucleotides will be desirable.

As used herein, oligonucleotide sequences that are complementary to one or more of the genes and/or gene families described in Tables 10-12, refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80 or 85% sequence identity or more preferably about 90 or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5 to 10% of the probes in the array, or, where a different background signal is calculated for each target gene, for the lowest 5 to 10% of the probes for each gene. Of course, one of skilled in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

The terms “mismatch control” or “mismatch probe” refer to a probe whose sequence is deliberately selected not to be perfectly complementary to a particular target sequence. For each mismatch (MM) control in a high-density array, there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe” or a “normalization control” probe, an expression level control probe and the like. A perfect match control or perfect match probe is, however, distinguished from a “mismatch control” or “mismatch probe” as defined herein.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (TO for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical residue (e.g., nucleic acid base or amino acid residue) occurs in both sequences to yield the number of matched positions, dividing the number of matched positions by the total number of positions in the window of comparison and multiplying the result by 100 to yield the percentage of sequence identity.

Percentage sequence identity can be calculated by the local homology algorithm of Smith & Waterman, (1981) Adv. Appl. Math. 2:482-485; by the homology alignment algorithm of Needleman & Wunsch, (1970) J. Mol. Biol. 48:443-445; or by computerized implementations of these algorithms (GAP & BESTFIT in the GCG Wisconsin Software Package, Genetics Computer Group) or by manual alignment and visual inspection.

Percentage sequence identity when calculated using the programs GAP or BESTFIT is calculated using dEPAult gap weights. The BESTFIT program has two alignment variables, the gap creation penalty and the gap extension penalty, which can be modified to alter the stringency of a nucleotide and/or amino acid alignment produced by the program. Parameter values used in the percent identity determination were dEPAult values previously established for version 8.0 of BESTFIT (see Dayhoff, (1979) Atlas of Protein Sequence and Structure, National Biomedical Research Foundation, pp. 353-358).

### Probe Design

One of skill in the art will appreciate that an enormous number of array designs are suitable for the practice of this invention. In some preferred embodiments, a high density array may be used. The high density array will typically include a number of probes that specifically hybridize to the sequences of interest, such as those described in Tables 10-12 (see WO 99/32660 for methods of producing probes for a given gene or genes). In addition, in a preferred embodiment, the array will include one or more control probes.

High density array chips of the invention include “test probes” as defined herein. Test probes may be oligonucleotides that range from about 5 to about 45 or 5 to about 500 nucleotides, from about 10 to about 40 nucleotides and from about 15 to about 40 nucleotides in length. The probes may be 20 or 25 nucleotides in length. Test probes may be double or single strand nucleic acid sequences, such as DNA sequences. Nucleic acid sequences may be isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Normalization probes may be selected to reflect the average length of the other probes present in the array, however, they can also be selected to cover a range of lengths. The normalization control(s) can also be selected to reflect the (average) base composition of the other probes in the array, however, only one or a few probes may be used and they may be selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Mismatch probes may contain a central mismatch. Thus, for example, where a probe is a twenty-mer, a corresponding mismatch probe will have the identical sequence except for a single base mismatch (e.g., substituting a G, C or T for an A) at any of positions six through fourteen (the central mismatch). Mismatch probes thus provide a control for non-specific binding or cross hybridization to a nucleic acid in the sample other than the target to which the probe is directed. Mismatch probes also indicate whether hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe provides a good measure of the concentration of the hybridized material.

### Nucleic Acid Samples

As is apparent to one of ordinary skill in the art, nucleic acid samples, which may be DNA and/or RNA, used in the methods and assays of the invention may be prepared by any available method or process. Methods of isolating total mRNA are well known to those of skill in the art. For example, methods of isolation and purification of nucleic acids are described in detail in Chapter 3 of Tijssen, (1993) Laboratory Techniques in Biochemistry and Molecular Biology: Hybridization With Nucleic Acid Probes, Elsevier Press. Such samples include RNA samples, but also include cDNA synthesized from an mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates can be used.

Biological samples may be of any biological tissue or fluid or cells from any organism as well as cells raised in vitro, such as cell lines and tissue culture cells. The sample may be a “clinical sample” which is a sample derived from a treated animal. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

### Forming High Density Arrays

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

### Hybridization

Nucleic acid hybridization simply involves contacting a probe and target nucleic acid under conditions where the probe and its complementary target can form stable hybrid duplexes through complementary base pairing (see WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA or RNA:DNA) will form even where the annealed sequences are not perfectly complementary. Thus specificity of hybridization is reduced at lower stringency. Conversely, at higher stringency (e.g., higher temperature and/or lower salt and/or in the presence of destabilizing reagents) successful hybridization tolerates fewer mismatches. One of skill in the art will appreciate that hybridization conditions may be selected to provide any degree of stringency. In a preferred embodiment, hybridization is performed at low stringency in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of destabilizing agents such as formamide. Hybridization specificity may be evaluated by comparison of hybridization to the test probes with hybridization to the various controls that can be present (e.g., expression level control, normalization control, mismatch controls, etc.).

In general, there is a trade-off between hybridization specificity (stringency) and signal intensity. Thus, the wash may be performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

### Signal Detection

The hybridized nucleic acids are typically detected by detecting one or more labels attached to the sample nucleic acids. The labels may be incorporated by any of a number of means well known to those of skill in the art (see WO 99/32660).

### Databases

The present invention includes relational databases containing sequence information, for instance, for the genes and members of the gene families disclosed herein as well as gene expression information in various tissue samples saved on computer readable medium and/or a user interface. Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequence database and a gene expression database. Methods for the configuration and construction of such databases are widely available, for instance, see U.S. Pat. No. 5,953,727, which is herein incorporated by reference in its entirety.

The databases of the invention may be linked to an outside or external database. In a preferred embodiment, the external database is GenBank and the associated databases maintained by the National Center for Biotechnology Information (NCBI).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic northern blots that allow the user to determine the cell type or tissue in which a given gene is expressed and to allow determination of the abundance or expression level of a given gene in a particular tissue or cell. The databases of the invention may also be used to present information identifying the expression level in a sample of a set of genes comprising one or more of the sequences of genes or members of the gene families disclosed herein, such as in Tables 10-12, comprising comparing the expression level of at least one gene or member of a gene family disclosed herein in the sample to the level of expression of the gene in the database.

### Diagnostic Uses

As described above, the genes and gene expression information described herein may be used as diagnostic markers for the efficacy of ginger in a diet. For instance, a tissue sample may be assayed by any of the methods described above, and the expression levels from a gene or member of a gene family described herein may be compared to the expression levels found in a non-ginger diet or an earlier point during a ginger diet. The comparison of expression data, as well as available sequence or other information may be done by researcher or diagnostician or may be done with the aid of a computer and databases as described above.

The present invention will be particularly useful by providing one or markers which may be used as markers for monitoring the progression and response to a ginger diet. The present invention may also be used in in vitro assays.

According to the present invention, the genes described herein may be used as markers to evaluate the effects of a test agent on a cell. A test agent can be screened for the ability to stimulate the transcription or expression of a given marker or markers or to down-regulate or counteract the transcription or expression of a marker or markers. For example, agents that modulate, induce or inhibit gene expression in a sample to that which resembles a gene expression profile following ingestion of a diet comprising at least 0.5% ginger by weight. As used herein, an agent is said to modulate the expression of a nucleic acid of the invention if it is capable of up- or down-regulating expression of the nucleic acid in a cell.

In one assay format, gene chips containing probes to one or more genes or members of gene families described herein may be used to directly monitor or detect changes in gene expression in the treated or exposed cell. In another format, cell lines that contain reporter gene fusions between the open reading frame and/or 5′→3′ regulatory regions of a gene or gene family member as described herein and any assayable fusion partner may be prepared. Numerous assayable fusion partners are known and readily available including the firefly luciferase gene and the gene encoding chloramphenicol acetyltransferase (Alam et al., (1990) Anal. Biochem. 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used, for instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al., (1989) Molecular Cloning: A Laboratory Manual, Cold Spring Harbor Laboratory Press.

In another assay format, cells or cell lines are first identified which express the gene products disclosed physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Such cells or cell lines may be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or some other detectable marker or tag. Such a process is well known in the art (see Sambrook et al., (1989) Molecular Cloning: A Laboratory Manual, Cold Spring Harbor Laboratory Press). Cells or cell lines transduced or transfected are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or western blot). The pool of proteins isolated from the “agent-contacted” sample is then compared with a control sample where only the excipient is contacted with the cells and an increase or decrease in the immunologically generated signal from the “agent-contacted” sample compared to the control is used to distinguish the effectiveness of the agent.

Another embodiment of the present invention provides methods for identifying agents that modulate the levels or at least one activity of a protein(s) encoded by the genes in Tables 10-12. Such methods or assays may utilize any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use of a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. An agent may be designed to have similar functional groups or shape as those found in ginger. The agents can be, as examples, peptides, small molecules, vitamin derivatives, as well as carbohydrates. Dominant negative proteins, DNA encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Meyers, (1995) Molecular Biology and Biotechnology, VCH Publishers, 659-664). A skilled artisan can readily recognize that there is no limit as to the structural nature of the agents of the present invention.

### Kits

The present invention also provides kits for assaying a gene profile. In another aspect, the present invention relates to kits which comprise:

(a) a polynucleotide of a gene disclosed herein or a fragment thereof;

(b) a nucleotide sequence complementary to that of (a);

(c) a polypeptide encoded by a gene disclosed herein, or a fragment thereof; or

(d) an antibody to a polypeptide encoded by a gene disclosed herein, or a fragment thereof.

It will be appreciated that in any such kit, (a), (b), (c) or (d) may comprise a substantial component. The manufacture of kits as described herein and components thereof (e.g., antibody production) may be achieved according to conventional methods.

This invention is not limited to the particular methodology, protocols, and reagents described herein because they may vary. Further, the terminology used herein is for the purpose of describing particular embodiments only and is not intended to limit the scope of the present invention. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compositions, compounds, methods, and similar information reported therein that might be used with the present invention. However, nothing herein is to be construed as an admission that the invention is not entitled to antedate such disclosure by virtue of prior invention.

In the specification there have been disclosed typical preferred embodiments of the invention and, although specific terms are employed, they are used in a generic and descriptive sense only and not for purposes of limitation, the scope of the invention being set forth in the following claims. Many modifications and variations of the present invention are possible in light of the above teachings. It is therefore to be understood that within the scope of the appended claims the invention may be practiced otherwise than as specifically described.

### SPECIFIC EMBODIMENTS OF THE INVENTION

The invention is further described in the following examples. The examples are merely illustrative and do not in any way limit the scope of the invention as described and claimed.

### Example 1

### Effect of Ginger in Cats with Chronic Diarrhea and Inflammatory Bowel Disease

The objective of this study was to determine the effect of ginger on the clinical symptoms in cats with chronic diarrhea or inflammatory bowel disease. Two groups of cats, a healthy control group and a test group of cats with chronic diarrhea or inflammatory bowel disease, were fed a baseline food for 2 weeks followed by the same baseline food containing 1% ginger root powder. Stool quality was measured daily.

TABLE 1
Nutrient analysis of control food and food with 1% ginger root powder

| Nutrient analysis | Control | Ginger |
|---|---|---|
| Moisture | 7.9 | 7.63 |
| Protein | 24.74 | 25.02 |
| Carbohydrate | 48.98 | 48.95 |
| Fat | 12.94 | 13.06 |
| Crude Fiber | 1.2 | 1.2 |
| Soluble Fiber | 1.2 | 1.3 |
| Insoluble Fiber | 5.6 | 5.8′ |
| DHA | <0.01 | <0.01 |
| EPA | <0.01 | <0.01 |
| Total omega 3 | 0.1 | 0.2 |
| Total omega 6 | 2.49 | 2.53 |
| Total tocopherols IU | 117.8 | 82.3 |
| Ascorbate/stay c (pg/g) | 37 | 20 |
| Calcium | 0.71 | 0.72 |
| Chloride-Soluble | 0.37 | 0.38 |
| Cysteine | 0.29 | 0.29 |
| Magnesium | 0.085 | 0.088 |
| Methionine | 0.63 | 0.67 |
| Phosphorus | 0.7 | 0.72 |
| Potassium | 0.55 | 0.55 |

Table 1 shows the nutrient content of the food. It was found that the food was very similar except for the incorporation of ginger in the food. There were no added fish oil or other antioxidants at levels that may be confounding in the results. Table 2 shows the frequency of stool quality of stools collected daily from the cats during the control food phase and the test food phase.

TABLE 2
The effect of ginger on stool quality in healthy cats and cats with
chronic diarrhea

| | | Stool quality frequency | | |
|---|---|---|---|---|
| | | (# of stools,/% of stools) | | |
| Clinical | | Stool Grades* | | |
| Subjects | Diet | 1, 2 | 3 | 4, 5 |
| IBD cats | Control food | 44 (26.2%) | 70 (41.7%) | 54 (32.1%) |
| | Ginger food | 23 (9.8%) | 111 (47.4%) | 100 (42.7%) |
| Normal cats | Control food | 2 (1.5%) | 15 (11.0%) | 11 (87.5%) |
| | Ginger food | 0 (0) | 17 (8.8%) | 176 (91.2%) |

*Stool quality is graded such that 1 and 2 are runny and watery, 3 is semi-formed and gel-like, and 4 and 5 are well formed to hard.)

It was found that by consuming the kibbles with added ginger cats with inflammatory bowel disease had significantly lower poor stools (9.8 vs. 26.2%) and higher good stools (42.7 vs. 32.1%). The food even slightly improved stool quality in normal cats although the stool quality was already very good. The healthy cats did not have any diarrhea episodes in the 3 week test period and had higher percentage of good stools (91% compared to 88%).

### Example 2

### Effect of Ginger on Stool Quality in Dogs with Chronic Diarrhea

The objective of this study is to determine the effect of ginger on the clinical symptoms in dogs with chronic diarrhea. Two groups of dogs, a healthy control group and a test group of dogs with chronic diarrhea were fed a baseline food for 2 weeks followed by the same baseline food containing 1% ginger root powder. Stool quality was measured daily.

TABLE 3
Nutrient analysis of control food and food with 1% ginger root powder

| Nutrient analysis | Control | Ginger |
|---|---|---|
| Moisture | 8.9 | 7.5 |
| Protein | 19.5 | 19.8 |
| Carbohydrate | 58 | 58.2 |
| Fat | 8 | 8.2 |
| Crude Fiber | 1.8 | 2.5 |
| Soluble Fiber | 0.6 | 1.6 |
| Insoluble Fiber | 7.9 | 8.6 |
| DHA | 0.01 | 0.01 |
| EPA | 0.01 | 0.01 |
| Total tocopherols IU | 61.2 | 64 |
| Ascorbate/stay c (pg/g) | 67.7 | 81 |
| Calcium | 0.67 | 0.68 |
| Chloride-Soluble | 0.36 | 0.41 |
| Cysteine | 0.25 | 0.26 |
| Magnesium | 0.1 | 0.1 |
| Methionine | 0.43 | 0.44 |
| Phosphorus | 0.6 | 0.6 |
| Potassium | 0.6 | 0.6 |

TABLE 4
The effect of ginger on stool quality in healthy dogs and dogs
with chronic diarrhea

| | | Stool quality frequency | | |
|---|---|---|---|---|
| | | (# of stools, % of stools) | | |
| Clinical | | Grades* | | |
| Subjects | Diets | 1, 2 | 3 | 4, 5 |
| CD dogs | Control food | 13 (11%) | 9 (8%) | 96 (81%) |
| | Ginger food | 14 (8%) | 9 (5%) | 155 (87%) |
| Normal dogs | Control food | 8 (7%) | 2 (2%) | 109 (92%) |
| | Ginger food | 2 (1%) | 7 (4%) | 187 (95%) |

*Stool quality is graded such that 1 and 2 are runny and watery, 3 is semi-formed and gel like, and 4 and 5 are well formed to hard.

The dietary intervention with 1% ginger resulted in an improvement in clinical symptoms in animals with chronic diarrhea or inflammatory bowel disease.

### Example 3

### Ginger in Canine and Feline Senior Diet

### Methods

Twenty senior cats and twenty senior dogs were utilized to determine the effects of ginger to improve markers of arthritis. In both cats and dogs, 10 had radiographic evidence of arthritis. A control food was fed for 14 days prior to feeding the control food+the test ingredient (Ginger). Ginger was fed for 28 days with samples taken at baseline (day 0) and then at study completion (day 28). Cartilage markers were measured in serum using ELISA kits and procedures to determine biomarker concentrations.

Data were analyzed using a two-way paired t-test to determine the change in marker levels with the hypothesis that the change is different from zero.

### Results

Table 5 shows arthritis biomarker values for ten normal and ten arthritic cats fed a control formula enhanced with 1% ginger for 28 days. With the exception of ratios analysis was performed on natural log transformed values. Means and SED are antilog values of log transformed data. Ratios were calculated and analyzed using untransformed data.

TABLE 5
Biomarker Values for Senior Cats

| Biomarker | Day 0 | Day 28 | SED | P-Value |
|---|---|---|---|---|
| CPII | 1650 | 1894 | 1.03 | 0.0004 |
| IL-1F | 376 | 312 | 1.06 | 0.0061 |
| IL-6F | 178 | 151 | 1.06 | 0.0173 |
| IFN-G | 37.9 | 29.7 | 1.14 | 0.0766 |

Table 6 shows arthritis biomarker values for ten normal and ten arthritic dogs fed canine arthritis control formula enhanced with 1% ginger for 28 days. Means, SED, and ratios were calculated and analyzed using untransformed data.

TABLE 6
Biomarker Values for Senior Dogs

| Biomarker | Day 0 | Day 28 | SED | P-Value |
|---|---|---|---|---|
| BAP | 24.99 | 26.15 | 0.78 | 0.1219 |
| NTX | 14.99 | 12.13 | 0.84 | 0.0458 |
| BAP:NTX Ratio | 1.81 | 2.21 | 0.10 | 0.0249 |

Table 7 shows a list of genes related to cartilage metabolism altered by ginger in canine immortalized cells

TABLE 7
Genes Related to Cartilage Metabolism

| | | Fold | |
|---|---|---|---|
| Gene | Probe | change | Function |
| HAS2 | CfaAffx.2351.1.S1_at | ↑ | hyaluronic acid synthesis, most |
| | | | predominant isoform in cartilage |
| Collagen type II A1 | Cfa.7192.1.A1_at | ↑ | The major component of articular |
| | | | cartilage |
| PGE2 | CfaAffx.24981.1.S1_at, | ↓ | Production of inflammatory products |
| | Cfa.6253.1.A1_at | | |
| Interluekin 6 | Cfa.3528.1.S1_s_at | ↓ | Inflammatory mediator |

### Example 4

### The Effect on Gene Expression of Three Levels of Ginger Root Powder in Canines

The effect on gene expression of three levels of ginger root powder was explored in a group of healthy beagles. The inclusion rate of ginger was 0.5%, 1.0%, and 1.5%. The experimental diets were fed for 6 days with blood collections at 0 and 6 days post-feeding. The data was compared to a basic control tool.

TABLE 8
Ingredients used in the Formulas

| Control | Ginger lx | Ginger 2x | Gimoer 3x |
|---|---|---|---|
| Corn | Corn | Corn | Corn |
| Poultry meal | Poultry meal | Poultry meal | Poultry meal |
| Grease | Grease | Grease | Grease |
| Pal Enhancer | Pal Enhancer | Pal Enhancer | Pal Enhances |
| Potassium Chloride | Potassium Chloride | Potassium Chloride | Potassium Chloride |
| Calcium Carbonate | Iodized Salt | Iodized Salt | Iodized Salt |
| Iodized Salt | Calcium Carbonate | Calcium Carbonate | Calcium Carbonate |
| Choline Chloride | Choline Chloride | Choline Chloride | Choline Chloride |
| Mineral Premix | Mineral Premix | Mineral Premix | Mineral Premix |
| Preservative | Preservative | Preservative | Preservative |
| Vitamin Premix | Vitamin Premix | Vitamin Premix | Vitamin Premix |
| | Ginger Root Powder | Ginger Root Powder | Ginger Root Powder |
| | | L-Tryptophan | L-Tryptophan |

TABLE 9
Diet Analysis

| Nutrient (100% Dry Matter | | | | |
|---|---|---|---|---|
| Basis) | Control | Ginger lx | Ginger 2x | Ginger 3x |
| Protein (%) | 18.50 | 19.99 | 19.88 | 19.82 |
| Animal Protein (%) | 11.53 | 13.24 | 13.17 | 13.16 |
| Fat (%) | 8.00 | 8.00 | 8.00 | 8.00 |
| Vitamin E (IU/G) | 0.18 | 0.18 | 0.18 | 0.18 |
| Total EPA (%) | 2.34 | 2.33 | 2.32 | 2.31 |
| n-3 Fatty acids | 0.09 | 0.09 | 0.09 | 0.09 |
| n-6 Fatty acids | 115 | 2.24 | 2.23 | 2.22 |
| Crude Fiber | 2.30 | 2.21 | 2.19 | 2.18 |

Forty healthy beagles were fed a control food from day 0 to day 24. The beagles were randomized in 4 groups (5 females, 5 males per group). One group remained on the control food while the other three received one of the experimental diets. On day 24 a blood sample was drawn prior to feeding the experimental diets in order to determine baseline gene expression and metabolomics. While on the treatment for the next six days, blood was drawn on day 30 in order to determine if the new food/ingredient had changed any gene expression. Total RNAs were isolated from whole blood samples using PAX gene RNA isolation kit. All measurements were done with the Canine 2 version of Affymetrix genechips. For statistical analysis, all measurements were normalized with RMA. ANOVA t-test was run. A cutoff at p<0.1 with fold change over 1.3 was used to generate the result. The following comparisons were made: 1× Ginger vs. Control, 2× Ginger vs. Control, and 3× Ginger vs. Control.

### Equipment and Materials

PAXgene Blood RNA tubes, PAXgene Blood RNA Kit, RNase Zap, Absolute ethanol, P1000 P200 P100 P10 and P2 Pipetman pipette, sterile filtered pipette tips, automatic pipet, pipet tips, test tube rack(s), Microcentrifuge tube rack(s), Permanent marker, Kimwipes®, Waste container (for PAXgene blood waste as Buffers BR2 and BR3 contain guanidine thiocyanate and therefore are not compatible with bleach), Eppendorf Microcentrifuge model #5417C, Beckman Coulter Allegra 25R Centrifuge, Thermomixer heater/shaker blocks, Vortex mixer, Ice and ice bucket. The PAXgene Blood RNA tubes and the PAXgene Blood RNA Kit are used together to isolate and purify intracellular RNA from whole blood obtained from both canines and felines. Blood is collected, using a Vacutainer® needle, directly into the PAXgene Blood RNA tube and then subjected to several rounds of centrifugation, wash and purification steps which ultimately result in high-quality RNA. The RNA then undergoes a quality control step and is then used in future quantitative real-time PCR and/or microarray analyses.

Assay Procedures:

1. Incubate PAXgene tubes (containing blood) for a minimum of 2 hours at room temperature before beginning the assay. If the tubes are frozen, and were not allowed to incubate for 2 hours prior to freezing, they will need to sit at room temperature to thaw and then an additional 2 hours.

2. Invert each PAXgene tube 8-10 times before the first centrifugation.

3. If using Buffer BR4 for the first time, add 4 volumes of 96-100% ethanol to the concentrated buffer to obtain α-working solution.

4. Preheat two heating blocks prior to beginning the assay to 65′C and 55′C.

5. Prepare the DNAase I stock solution (the RNase-Free DNase Set included with the PAXgene Blood RNA Kit)
5.1. Dissolve the solid DNase I enzyme in 550 μL of RNase-free water provided with the kit. Be sure not to lose any DNase I when removing the lid.
5.2. Mix gently by inverting the tube. DO NOT vortex or centrifuge.
5.3. Make a mixture of DNase I enzyme and Buffer RDD (enough volume for the number of samples being processed per batch). Each sample needs 704 of Buffer RDD and 10 μl, of DNase I (i.e. 20 samples would require a cocktail of 1.4 mL Buffer ROD and 200 μL DNase I). The cocktail should be stored at 2-8° C. until needed. The reconstituted enzyme is good for up to 6 weeks at 2-8° C.

1. RNA Isolation from Whole Blood:
1.1. Centrifuge the PAXgene Blood RNA tubes at 4000×g for 10 minutes.
1.2. Remove the supernatant by decanting and discard. Blot excess supernatant remaining on rim of PAXgene tube onto a Kimwipe. Add 4 mL of RNase-free water to the pellet and cap with a new Hemogard closure.
1.3. Resuspend the pellet by vortexing and centrifuge at 4000×g for 10 minutes.
1.4. Remove the supernatant by decanting and discard. Blot excess supernatant remaining on rim of PAXgene tube onto a Kimwipe.
1.5. Add 360 μL of Buffer BRI to the pellet and gently pipette until pellet is completely resuspended.
1.6. Transfer the sample to a sterile 1.5 mL microcentifuge tube and add 300 μL Buffer BR2 and 40 μL Proteinase K (do not mix Buffer BR2 and Proteinase K prior to adding to the sample). Mix each tube thoroughly by vortexing and place into the thermomixer preheated to 55° C. Incubate/shake the tubes for 10 minutes at 1400 rpm. Change the temperature of the thermomixers to 65° C. for step 3.6.
1.7. Pipet the lysate into a Q1Ashredder spin column placed into a 2 mL collection tube. Centrifuge at 14,000 rpm for 3 minutes.
1.8. Transfer the supernatant of the flow-through fraction to a sterile 1.5 mL microcentrifuge tuber.
1.9. Add 350 μL of 96-100% ethanol and gently mix by pipetting.
1.10. Add 700 μL of the sample to the PAXgene spin column placed in a 2 mL collection tube and centrifuge at 14,000 rpm for 1 minute. Transfer the PAXgene spin column into a new, 2 mL collection tube and discard the flow-through and old collection tube.
1.11. Add the remaining volume of the sample to the PAXgene spin column. Centrifuge at 14,000 rpm for 1 minute.

2. DNase I Treatment:

2.1. Discard the old collection tube and the flow-through (from step 1.11). Place the PAXgene spin column into a new 2 mL collection tube.

2.2. Add 350A of Buffer BR3 to the PAXgene spin column and centrifuge at 14,000 rpm for 1 minute. Discard the flow-through and collection tube.

2.3. Place the column into a new 2 mL collection tube and add 804 of the DNase (Buffer ROD cocktail (see “Assay Preparations”) directly to the column membrane and incubate for 15 minutes at room temperature.

2.4. Add another 350A Buffer BR3 to the PAXgene spin column. Centrifuge at 14,000 rpm for 1 minute. Transfer the PAXgene spin column to a new 2 mL collection tube and discard the old collection tube and flow-through.

3. Final RNA isolation:

3.1. Add 500 μL of Buffer BR4 to the PAXgene spin column. Centrifuge at 14,000 rpm fort minute. Place the PAXgene spin column into a new 2 mL collection tube and discard the old collection tube and flowthrough.

3.2. Add another 500 μL Buffer BR4 to the PAXgene spin column, Centrifuge at 14,000 rpm for 3 minutes to dry the PAXgene spin column membrane. Discard the collection tube and flow-through and place the columns in another 2 mL collection tube.

3.3. Spin the samples again at 14,000 rpm for an additional minute to further dry the column membrane. Discard the flow-through and the collection tube.

3.4. Transfer the PAXgene spin column to a 1.5 mL elution tube. Add 40 μL Buffer BR5 directly to the PAXgene spin column membrane. Centrifuge at 14,000 rpm for 1 minute.

3.5. Remove the PAXgene spin column and pipette the eluate in the 1.5 mL tube onto the same PAXgene column. Return the PAXgene spin column to the same 1.5 mL elution tube and centrifuge at 5000 rpm for 1 minute.

3.6. Incubate to the final eluate at 65° C. for 5 minutes and immediately chill on ice.

3.7. Store final RNA sample at −60° C. for future use.

Preparation of Labeled cRNA Probes Using Total Canine RNA Isolated from Whole Blood Samples

The isolated total RNA is can be used as a template to synthesize double-stranded cDNA using the One-Cycle cDNA Synthesis Kit (Affymetrix #900431). The cDNA is then purified and used as the template in the subsequent in vitro transcription reaction of cRNA and biotin labeling. The labeled cRNA is then cleaned, fragmented and ultimately hybridized to canine GeneChip® expression arrays.

### Materials

Purified canine or feline total RNA, 5 μg, One-Cycle cDNA Synthesis Kit, IVT Labeling Kit, Gene Chip Sample Cleanup Module, RNase Zap, Ethanol, Absolute, 200 proof, 80% Ethanol, prepared in RNase-free water, Sterile filtered pipette tips, 1.5 mL microcentrifuge tubes, 0.2 mL PCR tubes, sterile, microcentrifuge tube rack(s), PCR tube rack(s), P2, P10, P100, P200, P1000 Pipetman Pipets. Vortex, Peltier Thermal Cycler (PCR machine), Eppendorf Microcentrifuge, VWR Galaxy Mini Centrifuge, Incubator, 37′C

### Methods

### Day One

1. Synthesis of Double-Stranded cDNA from Total RNA
1.1. Determine the volume of total RNA that is equal to 5 μg of total RNA.
1.2. To a sterile 0.2 mL PCR tube add the following: 5 μg total RNA, DEPC-treated water, 2 μL T7-oligo(dT) primer, 50 μM—The total reaction volume=12 μL.
1.3. Incubate the samples at 70° C. for 10 minutes using the PCR machine.
1.4. Remove the samples from the PCR machine, quick spin and place on ice for 30 seconds to one minute.
1.5. Place the samples at room temperature and add the following to each of the samples; 4 μL 5× First-Strand cDNA buffer, 2 μL 0.1M DTT, 1 μL 10 mM dNTP mix, 1 μL SuperScript II RT. Mix quickly while samples are at room temperature. Incubate samples at 42° C. for 1 hour.

2. Second strand cDNA Synthesis
2.1. Remove the First-Strand reactions from the PCR machine and place on ice.
2.2. To each sample the following was added: 91 μL DEPC-treated water, 30 μL 5× Second-Strand Reaction Buffer, 3 μL 10 mM dNTP mix, 1 μL 10 U/μL E. coli DNA ligase, 4 μL 10 U/μL E. coli DNA Polymerase I, 1 μL 2 U/μL E. coli RNase H. Total reaction volume=150 μL.
2.3. Gently mix each sample and quick spin to remove condensation. Incubate samples at 16° C. for two hours using the PCR machine.
2.4. Remove the samples from the PCR machine and place on ice. Add 2 μL of (10 U) T4 DNA Polymerase to each sample.
2.5. Return the samples to 16° C. for 5 minutes.
2.6. Remove the samples from the PCR machine and add 10 μL of 0.5M EDTA to each sample.
2.7. Proceed to cleanup procedure for cDNA or store samples at −20° C. for later use.

3. Cleanup of Double-Stranded cDNA
3.1. Add 600 μL cDNA Binding Buffer to a sterile 1.5 mL microcentrifuge tube. Transfer the sample (162 μL) to the cDNA Binding Buffer in the new tube. Vortex for 3 seconds.
3.2. Check that the color of the mixture is yellow. (If the color is orange or violet, add 10 μL of 3M sodium acetate pH 5.0, and mix).
3.3. Apply 500 μL of the sample to a cDNA Cleanup Spin Column in a 2 mL Collection Tube, and centrifuge for 1 minute at 14,000 rpm. Discard flow-through.
3.4. Reload the spin column with the remaining volume (262 μL) of sample and repeat centrifugation as above. Discard flow-through and collection tube.
3.5. Transfer the spin column to a new 2 mL collection tube. Pipet 750 μL cDNA Wash Buffer onto the spin column. Centrifuge for 1 minute at 14,000 rpm. Discard flow-through.
3.6. Open the cap of the spin column and centrifuge samples for 5 minutes at 14,000 rpm. Discard flow through and collection tube.
3.7. Transfer the spin column to a 1.5 mL collection tube and pipet 14 μL of cDNA Elution Buffer directly onto the spin column membrane. Incubate the samples at room temperature for one minute. Centrifuge samples for 1 minute at 14,000 rpm.
3.8. Proceed to Synthesis of Biotin-Labeled cRNA or store samples at −20° C. for later use.

4. Synthesis of Biotin-Labeled cRNA
4.1. To the eluate from 3.7 add the following: (the approximate volume of the eluate is 12 μL) 8 μL RNase-free water, 4 μL 10× IVT Labeling Buffer, 12 μL IVT Labeling NTP mix, 4 μL IVT Labeling Enzyme mix. Total reaction volume=40 μL
4.2. Carefully mix the reagents and collect the contents at the bottom of the tube by a quick centrifugation.
4.3. Incubate the samples at 37° C. for 16 hours (overnight).
4.4. Proceed directly to Cleanup and Quantification of Biotin-Labeled cRNA or store at −20° C. for later use.

### Day Two

5. Cleanup of Biotin-Labeled cRNA
5.1. Add 60 μL RNase-free water to the in vitro transcription reaction and mix by vortexing for 3 minutes.
5.2. Add 350 μL IVT cRNA Binding Buffer to the sample and mix by vortexing for 3 seconds.
5.3. Add 250 μL pure ethanol to the lysate and mix by pipetting. DO NOT centrifuge.
5.4. Apply sample volume (700 μL) to the IVT cRNA Cleanup Spin Column in a 2 mL collection tube. Centrifuge for 15 seconds at 14,000 rpm. Discard the flow-through and collection tube.
5.5. Transfer the spin column to a new 2 mL collection tube. Pipet 500 μL IVT cRNA Wash Buffer onto the spin column. Centrifuge for 15 seconds at 14,000 rpm. Discard flow-through.
5.6. Add 500 μL 80% ethanol to the spin column and centrifuge for 15 seconds at 14,000 rpm. Discard flow through
5.7. Open the cap of each spin column and spin for an additional 5 minutes at 14,000 rpm. Discard flow through and the spin column.
5.8. Transfer the spin column to a new 1.5 mL collection tube. Pipet 21 μL of RNase-free water directly onto spin column membrane. Centrifuge for 1 minute at 14,000 rpm.
5.9 Select 1 μL aliquot of the sample for gel analysis.

6. Quantitation of cRNA
6.1. Use the NanoDrop spectrophotometer to determine the yield of cRNA.
6.2. If total RNA was used as the starting material uses the following equation to determine the adjusted cRNA yield: adjusted cRNA yield=RNAₘ−(total RNAᵢ)(y); RNAₘ=amount of cRNA measured after IVT (μg), total RNA=starting amount of total RNA, (μg) y=fraction of cDNA reaction used in IVT.

7. Fragmenting the cRNA for Target Preparation
7.1. Determine the sample volume needed to add 20 μg of cRNA and add the following to a 0.2 mL PCR tube: 20 μg cRNA, 8 μL 5× Fragmentation Buffer, RNase-free water. Total reaction volume=404.
7.2. Incubate the samples at 94° C. for 35 minutes. Put samples an ice following the incubation.
7.3. Save a 2 μL aliquot of the fragmented cRNA for gel analysis.
7.4. Store the undiluted fragmented cRNA at −20° C. until ready to proceed with hybridization.

8. Quality Control Gel Analysis
8.1. Combine both the pre-fragmentation and the fragmented cRNA (steps 5.8 and 7.3, respectively).
8.2. Run an RNA gel on the Agilent 2100 Bioanalyzer to verify the presence and quality of the cRNA product.

### Affymetrix GeneChip Expression Analysis

Total RNA is reverse transcribed into single, then double strand cDNA. The cDNA is used to generate cRNA, which is fragmented and used as probes for gene chip hybridization. The hybridization is washed and the hybridization signal is measured using an Affymetrix laser scanner. The hybridization data Is then validated and normalized for further genomic analysis. Please refer to the “GeneChip Expression Analysis Technical Manual” (701021 Rev. 5), Affymetrix, copyright 1999-2003

### Materials:

All reagents were purchased according to Affymetrix Manual. Please refer to “GeneChip Expression Analysis Technical Manual” (701021 Rev. 5) for the detail.

### Results

### Selected Genes affected by 1× Ginger

It appears that ginger has a pro-apoptotic effect in dogs, as genes such as Noclin (OLFM I) were up-regulated. In addition, Bcl-2 associated transcription factor (BCLAF1 or Btf) was up-regulated. Btf induces apoptosis, is involved in cell arrest and appears to have tumor suppressor activity.

Stress-70 protein (HSPA9B) was up-regulated. The gene product controls cell proliferation and cellular aging

Caspase 3 and 6 were up-regulated. Caspase 3 activates caspase 6.

Ribonucleoside-diphosphate reductase M2 subunit B (RRM2B) was up-regulated. The gene product is involved in DNA repair. It supplies deoxyribonucleotides for DNA repair in cells arrested at G1 or G2. The gene is induced by p53.

CARF (collaborates/cooperates with ARF (alternate reading frame)) was up-regulated. CARF enhances p53 functions. CARF may exert a vital role on p53-HDM2-p21 (WAF1) pathway that is central to cell cycle control, senescence, and DNA damage response of human cells.

Insulin-like growth factor-binding protein 6 precursor (IGFBP6) was up-regulated. The gene product binds and inhibits IGF-1 and IGF-2 and alters their interaction with surface receptors. Binding of IBP to IGF decreases cell survival and enhances apoptosis.

Vitamin Transporters were increased.

Transcobalamin (TCN I) was up-regulated. TCN I is a vitamin B12 binding protein. It facilitates the transport of cobalamin (Vit B12) into cells.

Retinol binding protein 4 (RBP4) was up-regulated. RBP4 binds and transports retinol.

One of the genes associated with immune response that was affected by 1× Ginger is ICOS. Inducible T-cell co-stimulator precursor (ICOS) was up-regulated. ICOS plays a role in development of cell help for B cells and in the generation, survival, and reactivation of memory CD4 T cells and B cells.

TABLE 10
Genes Affected by 1x Ginger

| | Fold Change | p-value | |
|---|---|---|---|
| | (1x Ginger vs. | (1x Ginger vs. | |
| Probeset ID | control day 6) | control day 6) | Gene Title |
| CfaAffx.2738.1.S1_at | −1.70 | 0.04402 | similar to BIA2 protein |
| CfaAffx.15740.1.S1_s_at | −1.62 | 0.05205 | similar to Hypothetical protein |
| | | | KIAA0481 (Cerebral protein-11) |
| | | | (hucep-11) |
| CfaAffx19070.1.S1_s_at | −1.59 | 0.08652 | — |
| Cfa.16256.1.S1_at | −1.49 | 0.07770 | LOC482842 |
| CfaAffx.2738.1.S1_s_at | −1.48 | 0.06066 | similar to BIA2 protein |
| CfaAffx.20590.1.S1_s_at | −146 | 0.08470 | similar to loss of heterozygosity, 12, |
| | | | chromosomal region 1 homolog |
| CfaAffx.17796.1.S1_at | −1.42 | 0.04738 | similar to CD1 antigen |
| Cfa.201.1.S1_at | −1.38 | 0.05509 | trefoil factor 2 |
| CfaAffx.2744.1.S1_at | −1.37 | 0.01484 | — |
| Cfa.4801.1.Al_at | −1.35 | 0.02424 | — |
| Cfa.5011.1.Al_at | −1.33 | 0.03247 | similar to Prostaglandin F2 receptor |
| | | | negative regulator precursor |
| | | | (Prostaglandin |
| CfaAffx.12550.1.S1_at | −1.32 | 0.02557 | similar to yippee-like 4 |
| CfaAffx.7340.1.S1_at | −1.31 | 0.02253 | similar to WO repeat. and FYVE |
| | | | domain-containing protein 2 |
| Cfa.12511.1.Al_at | 1.30 | 0.01353 | Transcribed locus |
| CfaAffx.23416.1.Sl_at | 1.30 | 0.05923 | similar to dJ54B20.4 (novel KRAB |
| | | | box containing C2H2 type zinc finger |
| | | | protein) |
| CfaAffx20207.1.Sl_at | 1.30 | 0.06163 | similar to dual specificity |
| | | | phosphatase 12 |
| Cfa,12994.1.Al_at | 1.30 | 0.09095 | Transcribed locus |
| CfaAffx.18525.1.S1_s_at | 1.30 | 0.01039 | similar to HEF-like protein |
| CfaAffx.919.1.S1_s_at | 1.30 | 0.04912 | — |
| Cfa.12712.1.S1_at | 1.31 | 0.05752 | similar to G elongation factor |
| CfaAffx.14786.1.S1_at | 1.31 | 0.09272 | LOG478761 |
| Cfa.10645.1.S1_at | 1.31 | 0.00383 | similar to ribosomal protein L18a |
| CfaAffx.21705.1.Sl_x_at | 1.31 | 0.04976 | similar to 60S ribosomal protein L21 |
| Cfa.17767.1.S1_at | 1.31 | 0.07274 | similar to eukaryotic translation |
| | | | elongation factor 1 alpha 1 |
| Cfa.3663.1.Al_s_at | 1.31 | 0.09646 | similar to immunoglobulin E receptor |
| | | | alpha chain |
| Cfa.21375.1.S1_s_at | 1.31 | 0.03942 | similar to ALL1 fused gene from |
| | | | 5q31 |
| Cfa.18816.1.S1_s_at | 1.31 | 0.02353 | similar to caspase 6 isoform alpha |
| | | | preproprotein |
| CfaAffx.30526.1.S1_s_at | 1.31 | 0.04554 | similar to hypothetical protein, |
| | | | DKFZp762E1712.1 - human |
| | | | (fragment) |
| Cfa.765.1.S1_s_at | 1.32 | 0.07798 | similar to receptor-interactinq factor 1 |
| Cfa.16441.1.A1_at | 1.32 | 0.03008 | Transcribed locus |
| Cfa.3073.1.Al_at | 1.32 | 0.09770 | Transcribed locus |
| CfaAffx438.1.S1_s_at | 1.32 | 0.06830 | similar to Keratin, type I cyloskeletal |
| | | | 18 (Cytokeratin 18) (K18) |
| | | | (CK18)1///si |
| Cfa.15902.1.A1_at | 1.32 | 0.04909 | similar to DNA methyltransferase 2 |
| | | | isoform b |
| Cfa.1204.2.S1_at | 1.32 | 0.09605 | Beta-spectrin |
| CfaAffx27633.1.S1_x_at | 1.32 | 0.00129 | similar to zinc finger protein 300 |
| Cfa.7678.1.Al_at | 1.32 | 0.00579 | similar to acyl-CoA:cholesterol |
| | | | acyltransferase 2 |
| Cfa.1184.1.S1_at | 1.32 | 0.08549 | Transcribed locus, weakly similar to |
| | | | NP 076943.1 hypothetical protein |
| | | | MGC2803 [H |
| Cfa.4769.1.A1_at | 1.32 | 0.01307 | — |
| Cfa.1896.1.S1_at | 1.32 | 0.03940 | Transcribed locus |
| Cfa.578.1.S1_at | 1.33 | 0.06757 | Transcribed locus |
| CfaAiix.1833.1.S1_at | 1.33 | 0.08699 | similar to ribonucleotide reductase |
| | | | M2 B (TP53 inducible) |
| CfaAffx.24940.1.S1_s_at | 1.33 | 0.08917 | similar to Rpl23a protein /// similar to |
| | | | ribosomal protein L23a /// similar to R |
| Cfa4ffx.19953.1.S1_s_at | 1.33 | 0.04692 | inducible T-cell co-stimulator |
| Cfa.12459.1.S1_s_at | 1.34 | 0.07641 | similar to 60S acidic ribosomal |
| | | | protein P2 /// similar to 60S acidic |
| | | | ribosomal p |
| Cfa.10824.1.A1_at | 1.34 | 0.03360 | similar to Dihydropteridine reductase |
| | | | (HDHPR) (Quinoid dihydropteridine |
| | | | reductase |
| Cfa.3846.1S1_x_at | 1.34 | 003384 | MHC class II DIA beta chain |
| | | | variable region (4-Pood) gene |
| CfaAffx.21346.1.S1_s_at | 1.34 | 0.08754 | similar to ribulose-5-phosphate-3- |
| | | | epimerase isoform 2 |
| Cfa.2869.1.A1_at | 1.34 | 0.08914 | Transcribed locus |
| CfaAftx.9192.1.S1_at | 1.34 | 0.03555 | similar to hypothetical protein |
| | | | MGC61571 |
| Cfa.13016.1.S1_at | 1.34 | 0.04925 | similar to Stress-70 protein, |
| | | | mitochondrial precursor (75 kDa |
| | | | glucose regulated |
| CfaAffx.3096.1.S1_x_at | 1.34 | 0.08460 | similar to 60S ribosomal protein |
| | | | L37a |
| CfaAffx.3587.1.S1_at | 1.34 | 0.00404 | similar to expressed sequence |
| | | | AW539457 |
| CfaAffx.12648.1.S1_s_at | 1.34 | 0.05136 | — |
| Cfa.17592.1.S1_at | 1.34 | 0.05420 | — |
| CfaAffx.8701.1.S1_at | 1.34 | 0.01618 | LOC475708 |
| Cfa 10068.1.A1_at | 1.34 | 0.05564 | similar to hypothetical protein |
| | | | FLJ10292 /// LOC488369 |
| CfaAffx.30939.1.S1_at | 1.34 | 0.04479 | similar to hypothetical protein |
| | | | DKFZp686G0786 |
| CfaAffx.7816.1.S1_at | 1.35 | 0.04137 | similar to Immunity-associated |
| | | | protein 4 (Immunity-associated, |
| | | | nucleotide 1 protein |
| CfaAffx.5937.1.S1_s_at | 1.35 | 0.09325 | — |
| Cfa.4155.1-A1_at | 1.35 | 0.08332 | Transcribed locus |
| Cfa.21582.1.S1_s_at | 1.35 | 0.05731 | similar to RIKEN cDNA |
| | | | 1200015A22 |
| CfaAffx.6478.1.S1_s_at | 1.35 | 0.09179 | similar to Suppressor of G2 allele of |
| | | | SKP1 homolog (Sgt1) (Putative 40-6- |
| | | | 3 prote |
| CfaAffx.15773.1.S1_at | 1.35 | 0.04393 | — |
| CfaAffx.21377.1.S1_at | 1.35 | 0.02855 | similar to Beta-ureidopropionase |
| | | | (Beta-alanine synthase) (N- |
| | | | carbamoyl-beta-alanine |
| CfaAffx.705.1.S1_at | 1.35 | 0.07560 | similar to T cell receptor beta chain |
| | | | hcvbl2 |
| Cfa.6911.1.A1_s_at | 1.35 | 0.09601 | LOC475030 |
| Cfa.12478.1.S1_at | 1.35 | 0.04047 | — |
| Cfa.14608.1.A1_at | 1.36 | 0.04218 | Transcribed locus |
| Cfa.13956.1.A1_at | 1.36 | 0.08911 | Transcribed locus |
| Cfa.20847.1.S1_s_at | 1.36 | 0.05286 | — |
| CfaAffx.19953.1.S1_at | 1.36 | 0.02242 | — |
| Cfa.2114.1.S1_at | 1.36 | 0.08787 | similar to Hypothetical protein |
| | | | FLJ11184 |
| Cfa.2604.1.S1_at | 1.37 | 0.07995 | Transcribed locus |
| Cfa.10174.3.A1 a at | 1.37 | 008901 | similar to mitochondrial cytochrome |
| | | | c oxidase subunit 5a |
| Cfa.3966.1.S1_at | 1.37 | 007705 | Transcribed locus |
| Cta.9971.1.S1_at | 1.38 | 0.06940 | similar to lactamase, beta 2 |
| Cfa.21584.1.S1_s_at | 1.38 | 0.05002 | LOC483999 |
| CfaAffx.25331.1.S1_at | 1.39 | 0.08248 | similar to aquaporin 9 |
| CfaAffx.2490.1.S1_at | 1.40 | 0.00319 | similar to 60S ribosomal protein L17 |
| | | | (L23) |
| Cfa.2087.1.A1_at | 1.40 | 0.06795 | similar to ligatin |
| CfaAffx.11510.1.S1_at | 1.40 | 001081 | similar to acyl-CoA:cholesterol |
| | | | acyltransferase 2 |
| CfaAffx.7767.1.S1_s_at | 1.41 | 0.04776 | similar to DNA methyltransferase 2 |
| | | | isoform b |
| CfaAffx. | 1.41 | 0.02872 | similar to Keratin, type 1 cytoskeletal |
| 11632.1.S1_s_at | | | 18 (Cytokeratin 18) (K18) (CK18) |
| CfaAffx.19115.1.S1_at | | | similar to Synaptonemal complex |
| | | | protein 2 (SCP-2 protein) |
| Cfa.10306.1.S_at | 1.41 | 0.00372 | similar to olfactomedin I |
| Cfa.8042.1.A1_at | 1.42 | 0.05558 | LOC475708 |
| Cfa.10795.1.A1_at | 1.42 | 0.05133 | Transcribed locus, moderately similar |
| | | | to XP508581.1 |
| | | | PREDICTED: similar to CAAX |
| CfaAffx.16182.1.S1_at | 1.42 | 0.01096 | — |
| Cfa.18816.1.S1_at | 1.43 | 0.01648 | similar to caspase 6 isoform alpha |
| | | | preproprotein |
| CfaAff,.23800.1.S1_s_at | 1.45 | 0.02168 | similar to hypothetical protein |
| | | | MGC35274 |
| Cfa.3955.1.A1_s_at | 1.46 | 0.04710 | similar to HIV-1 Rev binding protein |
| | | | 2 (Rev interacting protein 1) (Rip-1) |
| CfaAffx.21059.1.S1_at | 1.47 | 0.01120 | similar to immunoglobulin lambda |
| | | | chain |
| Cfa.8912.1.A1_at | 1.47 | 0.03058 | Transcribed locus |
| Cfa.5372.1.A1_at | 1.49 | 0.05832 | — |
| Cfa-11280.1.A1_at | 1.50 | 0.04232 | similar to hypothetical protein |
| | | | B230397C21 |
| Cfa.3402.2.A1_at | 1.51 | 0.02038 | Transcribed lows. strongly similar-to |
| | | | XP_346695.1 PREDICTED: |
| | | | hypothetical protein |
| Cfa.5819.1.A1_s_at | 1.52 | 0.02531 | — |
| Cfa.15489.1.S1_at | 1.52 | 0.07943 | similar to Plasma retinol-binding |
| | | | protein precursor (PREP) (RBP) |
| | | | (PRO2222) |
| CfaAffx.16925.1.S1_at | 1.56 | 0.03696 | similar to lysophospholipase-like 1 |
| Cfa.19957.1.S1_s_at | 1.56 | 0.01662 | LOC475675 |
| CfaAffx.14564.1.S1_at | 1.56 | 0.05592 | similar to round spermatid basic |
| | | | protein 1 |
| Cla.16M.A1_at | 1.63 | 0.0140 | Transcribed locus |

### Selected Genes affected by 2× Ginger

### Cell Integrity and DNA Replication Genes

Talin (TLN 1) was down-regulated. Talin is a high-molecular weight cytoskeletal protein concentrated at region of cell-substratum contact. It links actin cytoskeleton to extracellular environment. With reduced talin there will be less cell to cell contact in oocytes, integrin signaling will be inhibited, and cells will be less invasive, i.e. talin may have some tumor suppression effects.

Topoisomerase (TOP2A) was down-regulated. This enzyme is involved in DNA function. It functions as a target for several anticancer agents. DNA Primase (PRIM1) was down-regulated. TOP2A is involved in DNA replication.

### Anti-Inflammatory Genes

Interleukin-1 receptor type 11 (IL1 R2) was up-regulated. This IL-1 receptor does not contribute to IL-1 signaling. It acts as a decoy for IL-1 and inhibits IL-1 activity and is a specific down-regulator of IL-1 action.

Multisynthetase complex auxiliary component p43 contains endothelial monocyte-activating polypeptide 2 (EMAPII or SCYE) was down-regulated with ginger. The EMAPII product has inflammatory cytokine activity. It is induced by apoptosis.

Natural cytotoxicitytriggering receptor 3 (NCR3) was down-regulated with ginger. This gene is up-regulated in rheumatoid arthritis, with LPS, INF-gamma, or bacterial infection.

Interleukin-18 binding protein precursor (IL-18BP) was up-regulated with ginger. IL-18 binding protein binds IL-18 and inhibits its functions. It is an inhibitor of early TH1 cytokine response. It inhibits IL-18-induced INF-gamma production, and has been shown to have anti-inflammatory effect in skin inflammation.

Vitamin Transporter were Increased

Transcobalamin (TCN I) was up-regulated. TCN I is a vitamin B12 binding protein. It facilitates the transport of cobalamin (Vit B12) into cells.

Cubilin (CUBN) was up-regulated. Cubilin is important for Vitamin B12 absorption and for reabsorption of proteins. It is essential for renal tubular uptake of albumin. Cubilin serves as receptor for intrinsic factor B12 complex. Dysfunctional Cubilin affects cellular uptake and metabolism of steroid hormones, e.g., vitamin D3.

### Collagen Synthesis Genes

Prolyl 4-hydroxylase alpha-I and alpha-2 subunit precursor (P4HA) were up-regulated with ginger. The active enzyme is a tretramer of 2 alpha and 3 beta subunits. This enzyme catalyzes posttranslational formation of 4-hydroxyproline in collagen and other proteins. It has pro collagen-proline 4-dioxygenase activity.

### Other Genes of Interest

Calreticulin precursor (CALR) was down-regulated with ginger. The gene product is a Calcium binding/storage protein located in the ER and in nucleus. CALR can inhibit the binding of glucocorticoid receptor, androgen receptors to their response elements. CALR acts as important modulator of regulation of gene transcription by nuclear hormone receptors. This gene is up-regulated in colorectal cancer and liver cancer. Calreticulin is increased on the surface of apoptotic cells and interacts with adiponectin during clearance of apoptotic cells.

TABLE 11
Genes Affected by 2x Ginger

| | Fold Change | p-value | |
|---|---|---|---|
| | (2x Ginger vs. control | (2x Ginger vs. control | |
| ProbesetID | day 6) | day 6) | Gene Title |
| CfaAffx.265.1.S1_s_at | −1.72 | 0.02118 | similar to immunoglobulin V |
| | | | lambda chain |
| Cfa.12147.1.A1_s_at | −1.49 | 0.03873 | similar to KIAA1027 protein |
| CfaAffx.22074.1.S1_s_at | −1.46 | 0.07676 | similar to Radixin (ESP10) |
| Cfa.19700.1.S1_s_at | −1.44 | 0.00979 | similar to Nuclear transport factor |
| | | | 2 (NTF-2) |
| Cfa.18946.1.S1_s_at | −1-39 | 0.02018 | similar to topoisomersae II |
| CfaAffx.19053.1.S1_s_at | −1.38 | 0.04912 | similar to Uncharacterized |
| | | | hematopoietic |
| | | | stem/progenitor cells protein |
| | | | MDS029 // |
| CfaAffx.10449.1.S1_s_at | −1.38 | 0.03043 | — |
| CfaAf x.1729.1.S1_at | −1.37 | 0.03589 | natural cytotoxicity triggering |
| | | | receptor 3 |
| Cfa.4277.1.A1_s_at | −1.37 | 0.02034 | similar to calreticulin precursor, |
| | | | skeletal muscle - rabbit |
| CfaAffx.19651.1.S1_s_at | −1.37 | 0.04109 | similar to Serine-threonine kinase |
| | | | receptor-associated protein (UNR- |
| | | | interacting |
| CfaAffx.13689.1.S1_at | −1.37 | 0.01975 | similar to SAM domain and HD |
| | | | domain-containing protein 1 |
| | | | (Dendritic cell-derived |
| Cfa.17994.1.S1_s_at | −1.37 | 0.02215 | similar to male sterility domain |
| | | | containing 1 |
| Cfa.579.1.A1_at | −1.36 | 0.01252 | similar to DNA primase small |
| | | | subunit (DNA primase 49 kDa, |
| | | | subunit) (p49) |
| Cfa.12128.1.A1_at | −1.36 | 0.08613 | Transcribed locus |
| CfaAffx.7724.1.S1_at | −1.36 | 0.00352 | similar to Uncharacterized |
| | | | hypothalamus protein HT007 |
| CfaAffx.17815.1.S1_s_at | −1.35 | 0.06368 | similar to 26S proteasome non- |
| | | | ATPase regulatory subunit 12 |
| Cfa.4113.1.S1_at | −1.35 | 0.06152 | Transcribed locus, strongly similar |
| | | | to NP_002406.1 macrophaqe |
| | | | migration inhibitor |
| Cfa.11727.1.A1_s_at | −1.35 | 0.04933 | similar to SECIS-binding protein 2 |
| | | | (Selenocysteine insertion sequence |
| | | | binding protein |
| Cfa.15678.1.A1_at | −1.34 | 0.01674 | Transcribed locus |
| Cfa.21464.1.S1_s_at | −1.34 | 0.00888 | similar to calreticulin precursor. |
| | | | skeletal muscle - rabbit |
| Cfa.21258.1.S1_at | −1.33 | 0.08778 | similar to CD16 |
| Cfa.6255.1.S1_s_at | −1.32 | 0.00305 | — |
| Cfa.168.1.S1_at | −1.32 | 0.06697 | basic helix-loop-helix domain |
| | | | containing, class B3 |
| CfaAffx.31149.1.S1_at | −1.32 | 0.04433 | similar to Nuclear transport factor |
| | | | 2 (NTF-2) /// similar to Nuclear |
| | | | transport f |
| Cfa.20063.1.S1_s_at | −1.31 | 0.07189 | similar to kinectin |
| Cfa.1780.1.S1_at | −1.31 | 0.07842 | similar to flap structure-specific |
| | | | endonuclease 1 |
| CfaAffx.28712.1.S1_at | −1.31 | 0.07247 | similar to Ribosome biogenesis |
| | | | protein Brix |
| CfaAffx.1697.1.S1_s_at | −1.31 | 0.05636 | MHC class I DLA-88 /// MHC |
| | | | class I DLA-12 |
| Cfa.8028.1.S1_s_at | −1.31 | 0.03409 | similar to Small nuclear |
| | | | ribonucleoprotein F (snRNP-F) |
| | | | (Sm protein F) (Sm-F) (Sm |
| CfaAffx.21325.1.S1_at | −1.30 | 0.04338 | — |
| CfaAffx.12454.1.S1_s_at | −1.30 | 0.08404 | caspase-3 |
| Cfa.4331.2.S1_at | 1.30 | 0.04659 | LOC476145 |
| CfaAffx.13018.1.S1_at | 1.30 | 0.00322 | similar to hypothetical protein |
| | | | 5832424M12 |
| Cfa.20568.1.S1_at | 1.30 | 0.05781 | similar to sortilin-related receptor |
| | | | containing LDLR class A repeats |
| | | | preproprote |
| Cfa.2541.1.A1_at | 1.30 | 0.08750 | — |
| CfaAffx.12274.1.S1_at | 1.31 | 0.03173 | similar to CMP-N- |
| | | | acetylneuraminate-poly-alpha-2,8- |
| | | | sialyl transferase (Alpha-2,8- |
| Cfa.18524.1.S1_at | 1.31 | 0.05712 | LOC488975 |
| CfaAffx.1755.1.S1_at | 1.31 | 0.06975 | — |
| AFFX-BioC-3_at | 1.31 | 0.08831 | — |
| Cfa.15559.1.A1_at | 1.32 | 0.09709 | Transcribed locus |
| Cfa.14225.1.A1_s_at | 1.32 | 0.09340 | similar to protein phosphatase 1. |
| | | | regulatory subunit 15A |
| Cfa.3846.1.S1_x_at | 1.32 | 0.03973 | MHC class II DLA beta chain |
| | | | variable region (4-Pood) gene |
| Cfa.1612.1.A1_at | 1.33 | 0.06342 | Transcribed locus |
| Cfa.11266.1.A1_at | 1.33 | 0.06477 | similar to acylphosphatase (EC |
| | | | 3.6.1.7), skeletal muscle-pig |
| Cfa13370.1.A1_at | 1.34 | 0.04214 | similar to CD68 |
| Cfa.2353.1.A1_at | 1.34 | 0.00525 | similar to hypothetical protein |
| | | | MGC48625 |
| Cfa.14516.1.S1_at | 1.35 | 0.02325 | similar to II 18bp protein |
| Cfa.20138.1.S1_s_at | 1.36 | 0.09180 | similar to DnaJ homolog subfamily |
| | | | B member 1 (Heat shock 40 kDa |
| | | | protein 1) (Heat |
| Cfa.21239.1.S1_s_at | 1.36 | 0.07999 | similar to Prolyl 4-hydroxylase |
| | | | alpha-1 subunit |
| | | | precursor (4-PH alpha-1) (Procol |
| CfaAffx.1833.1.S1_at | 1.36 | 0.06194 | similar to ribonucleotide reductase |
| | | | M2 B (TP53 inducible) |
| CfaAffx_16182.1.S1_at | 1-37 | 0.02413 | — |
| Cfa.12478.1.S1_at | 1.37 | 0.03491 | — |
| Cfa.7527.1.A1_at | 1.37 | 0.00908 | Transcribed locus |
| Cfa.15625.1.A1_at | 1.37 | 0.09459 | Transcribed locus |
| CfaAffx.5057.1.S1_s_at | 1.37 | 0.01676 | similar to hypothetical protein |
| | | | MGC48625 |
| Cfa.5221.1.A1_s_at | 1.37 | 0.02250 | similar to interleukin-1 receptor |
| | | | type II |
| Cfa.19421.1.A1_at | 1.37 | 0.02990 | Transcribed locus |
| Cfa.15478.1.A1_at | 1.38 | 0.00367 | similar to 6-phosphofructo-2- |
| | | | kinase heart isoform |
| CfaAffx.4122.1.S1_at | 1.38 | 0.01315 | similar to Interleukin-1 receptor, |
| | | | type II precursor (IL-1R-2) (IL-1R- |
| | | | beta) (Ant |
| CfaAffx.17255.1.S1_at | 1.38 | 0.03852 | — |
| CfaAffx.5058.1.S1_s_at | 1.38 | 0.03380 | similar to hypothetical protein |
| | | | MGC48625 |
| CfaAffx.15773.1.S1_at | 1.39 | 0.02864 | — |
| Cfa.19361.1.S1_s_at | 1.39 | 0.00168 | LOC476127 |
| Cfa.16629.1.A1_at | 1.39 | 0.04023 | Transcribed locus |
| Cfa.5834.1.S1_at | 1.39 | 0.04682 | Transcribed locus |
| Cfa.9150.1.A1_at | 1.40 | 0.02403 | Transcribed locus |
| Cfa.14817.1.A1_at | 1.41 | 0.00260 | similar to Amyotrophic lateral |
| | | | sclerosis 2 chromosomal region |
| | | | candidate gene pro |
| Cfa.1625.1.A1_at | 1.42 | 0.07292 | Transcribed locus |
| CfaAffx.8344.1.S1_s_at | 1.44 | 0.06516 | — |
| Cfa.3631.1.A1_at | 1.51 | 0.06367 | Cubilin |
| Cfa.10576.1.A1_at | 1.57 | 0.01320 | Transcribed locus. moderately |
| | | | similar to XP_535316.1 |
| Cfa.13766-1.A1_at | 1.60 | 0.00759 | Transcribed locus |
| CfaAffx.19115.1.S1_at | 1.60 | 0.01824 | similar to Synaptonemal complex |
| | | | protein 2 (SCP-2 protein) |
| | | | (Synaptonemal complex |
| Cfa.20847.1.S1_s_at | 1.61 | 0.00316 | — |

### Selected Genes Affected by 3× Ginger

### Apoptosis and Growth Arrest Genes

Stress-70 protein (HSPA9B) was up-regulated. The gene product controls cell proliferation and cellular aging.

Caspase 6 (CASP6) was up-regulated. Over-expression of caspase 6 promotes apoptosis.

Baculoviral IAP repeat-containing protein 3 (BIRC3: c-IAP2) was up-regulated with ginger. The gene product acts as an apoptotic suppressor.

Growth arrest and DNA-damage-inducible protein 34 (GADD34), regulatory (inhibitor) subunit 15A (PPPIR15A) was up-regulated with ginger. This gene is a member of a group of genes whose transcript levels are increased following stressful growth arrest conditions, treatment with DNA-damaging agents, heat shock, nutrient deprivation, energy depletion, and ER stress. It inhibits viral replication via inhibition of the mTOR pathway. GADD34 is a protein that plays a pivotal role in the recovery of cells from shut-down of protein translation induced by ER stress. GADD34 contributes to the regulation of p21 expression. It suppresses cellular proliferation through the induction of cellular senescence.

W W domain-containing oxidoreductase (W WOX) was down-regulated. Probable oxidoreductase, which may act as a tumor suppressor and plays a role in apoptosis. May function synergistically with p53 to control genotoxic stress-induced cell death. Essential mediator of TNF-alpha-induced apoptosis.

### Cell Integrity. Integrin Signaling, and Apoptosis Genes

Talin (TLN1) was down-regulated. Talin is a high-molecular weight cytoskeletal-protein concentrated at region of cell-substratum contact. It links actin cytoskeleton to extracellular environment. With reduced talin there will be less cell to cell contact in lymphocytes, integrin signaling will be inhibited, and cells will be less invasive, i.e., reduced talin may have some tumor suppression effects.

Caspase 6 (CASP6) was up-regulated. Caspase 6 is involved in the activation cascade of caspases responsible for apoptosis execution. >Over-expression promotes programmed cell death.

### Kidney Cancer, Cholesterol and Fatty Acid Biosynthesis Genes

Ring finger protein 139 (TRCB=RNF139) was up-regulated. This gene is a potential tumor suppressor gene for renal cell carcinoma, The gene product is located at the ER and has been shown to possess ubiquitin ligase activity, The protein may interact with the tumor suppressor protein von Hippel-Lindau (VHL). Expression of TRC8 represses genes involved in cholesterol and fatty acid biosynthesis that are transcriptionally regulated by the sterol response element binding proteins (SREBPs).

### Collagen Synthesis

Prolyl 4-hydroxylase alpha-I and alpha-2 subunit precursor (P4HA) were up-regulated with ginger. The active enzyme is a tretramer of 2 alpha and 3 beta subunits. This enzyme catalyzes post-translational formation of 4-hydroxyproline in collagen and other proteins. It has pro collagen-proline 4-dioxygenase activity, i.e., this enzyme is important for collagen synthesis.

### Vitamin Transporter Genes

Cubilin (CUBN) was up-regulated. Cubilin is important for Vitamin B12 absorption and reabsorption of proteins. It is essential for renal tubular uptake of albumin. CUBN serves as receptor for intrinsic factor B 12 complex. Dysfunctional Cubilin affects cellular uptake and metabolism of steroid hormones, e.g., vitamin D3.

TABLE 12
Genes Affected by 3x Ginger

| | Fold Change | p-value | |
|---|---|---|---|
| | (3x Ginger vs. | (3x Ginger vs. | |
| Probeset ID | control day 6) | control day 6) | Gene Title |
| Cfa.10558.3.A1_at | −2.53 | 0.07285 | Transcribed locus, strongly similar to |
| | | | NP 003842.1 cellular repressor of |
| | | | ElA-sti |
| CfaAffx.265.1.S1_s_at | −1.63 | 0.03858 | similar to immunoglobulin V lambda |
| | | | chain |
| CfaAffx.5333.1.S1_at | −1.57 | 0.07465 | similar to thymocyte protein thy28 |
| | | | isoform 1 |
| CfaAffx.18690.1.S1_s_at | −1.48 | 0-02846 | similar to transglutaminase X |
| Cfa.12147.1.A1_s_at | −1.40 | 0.07896 | similar to KIAA 1027 protein |
| CfaAffx.14486.1.S1_at | −1.35 | 0.05179 | LOC487948 |
| CfaAffx.17796.1-S1_at | −1.34 | 0.09521 | similar to CD1 antigen |
| Cfa.12234.1.A1_at | −1.32 | 0.03941 | similar to hypothetical protein |
| | | | FLJ23221 |
| CfaAffx.30659.1.S1_s_at | −1.31 | 0.00009 | similar to W W domain-containing |
| | | | oxidoreductase isoform 2 |
| Cfa.15678.1.A1_at | −1.31 | 0.02961 | Transcribed locus |
| Cfa.16280.1.S1_at | −1.30 | 0.00729 | — |
| CfaAffx.309.1.S1_x_at | 1.30 | 0.01861 | similar to ribosomal protein L31 |
| Cfa1453.1.A1_at | 1.30 | 0.09125 | Transcribed locus |
| CfaAffx.161151.S1_s_at | 1.30 | 0.01740 | similar to RIKEN cDNA |
| | | | 2010012O05 |
| Cfa.1201.1.A1 s at | 1.31 | 0.01941 | Ribosomal protein S17 |
| Cfa.14989.2.A1_a_at | 1.31 | 0.02208-, | similar to zinc finger. CSL domain |
| | | | containing 3 |
| Cfa.10278.1.A1_at | 1.31 | 0.07589 | similar to ribosomal protein L24-like |
| Cfa.1447.1.A1_at | 1.31 | 0.07544 | Transcribed locus |
| Cfa.16446.1.A1_s_at | 1.31 | 0.00274 | LOC475649 |
| Cfa.13016.1.S1_at | 1.32 | 0.06322 | similar to Stress-70 protein, |
| | | | mitochondrial precursor (75 kDa |
| | | | glucose regulated |
| Cfa.10645.1.S1_at | 1.32 | 0.00302 | similar to ribosomal protein L18a |
| Cfa.14562.1.A1_at | 1.32 | 0.08335 | similar to paraoxonase 3 |
| Cfa.17941_1.S1_s_at | 1.32 | 0.09626 | similar to dual specificity |
| | | | phosphatase 12 |
| CfaAffx.24250.1.S1_s_at | 1.32 | 0.00769 | similar to RIKEN cDNA |
| | | | 2310005NO3 |
| Cfa1184.1.S1_at | 1.32 | 0.08375 | Transcribed locus, weakly similar to |
| | | | NP_076943.1 hypothetical protein |
| | | | MGC2803 (H |
| Cfa1863.1.A1_at | 1.32 | 0.00115 | Transcribed locus, weakly similar to |
| | | | XP_537697.1 PREDICTED: similar |
| | | | to KIAA0898 |
| CfaAffx.2490.1.S1_at | 1.33 | 0.01128 | similar to 60S ribosomal protein L17 |
| | | | (L23) |
| CfaAffx.6741.1.S1_at | 1.33 | 0.08263 | similar to taste receptor type 2 |
| | | | member 38 |
| Cfa.18376.1.S1_at | 1.34 | 0.04361 | similar to Baculoviral IAP repeat- |
| | | | containing protein 3 (Inhibitor of |
| | | | apoptosis p |
| Cfa.10136.2.S1_a_at | 1.34 | 0.00901 | similar to Methionine-R-sulfoxide |
| | | | reductase B (CGI-131) |
| Cfa.15902.1.A1_at | 1.34 | 0.03841 | similar to DNA methyltransferase 2 |
| | | | isoform b |
| CfaAffx.4313.1.S1_at | 1.34 | 0.08832 | similar to hypothetical protein |
| | | | BC018453 |
| CfaAffx.1755.1.S1_at | 1.34 | 0.04882 | — |
| Cfa.10068.1.A1_at | 1.34 | 0.05712 | similar to hypothetical protein |
| | | | FLJ10292 /// LOC488369 |
| Cfa.20853.1.S1_s_at | 1.34 | 0.09996 | similar to U6 snRNA-associated Sm- |
| | | | like protein LSm8 /// similar to U6 |
| | | | snRNA-asso |
| Cfa.21375.1.S1_s_at | 1.34 | 0.02546 | similar to ALL1 fused gene from |
| | | | 5q31 |
| Cfa.15469.1.A1_s_at | 1.34 | 0.07000 | — |
| CfaAffx.19831.1.S1_x_at | 1.34 | 0.03476 | similar to ribosomal protein L31 |
| Cfa.19151.1.A1_at | 1.35 | 0.06377 | similar to hypothetical protein |
| | | | D11Ertd497e |
| Cfa.16431.1.A1_at | 1.35 | 0.06494 | Transcribed locus |
| CfaAffx.13305.1.S1_s_at | 1.35 | 0.07450 | — |
| Cfa.511.1.S1_at | 1.35 | 0.07097 | Transcribed locus |
| CfaAffx.21377.1.S1_at | 1.36 | 002653 | similar to Beta-ureidopropionase |
| | | | (Beta-alanine synthase) (N- |
| | | | carbamoyl-beta-alani |
| Cfa.1838.1.A1_at | 1.36 | 009683 | — |
| Cfa.2470.1.A1_at | 1.36 | 001482 | Transcribed locus, moderately |
| | | | similar to XP_372753.2 |
| | | | PREDICTED: similar to Zinc |
| Cfa.2848.2.S1_x_at | 1.36 | 0.00819 | similar to hypothetical protein |
| | | | MGC5306 /// similar to hypothetical |
| | | | protein MGC5 |
| CfaAffx.23416.1.S1_at | 1.36 | 0.02642 | similar to dJ54B20.4 (novel KRAB |
| | | | box containing C2H2 type zinc |
| | | | finger protein) |
| CfaAffx.22481.1.S1_at | 1.36 | 0.07380 | similar to Prolyl 4-hydroxylase |
| | | | alpha-1 subunit precursor (4-PH |
| | | | alpha-1) (Procol |
| Cfa.12478.1.S1_at | 1.37 | 0.03519 | — |
| Cfa.765.1.S1_s_at | 1.37 | 0.04557 | similar to receptor-interacting factor 1 |
| CfaAffx.15773.1.S1_at | 1.37 | 0.03587 | — |
| CfaAffx.705.1.S1_at | 1.37 | 0.06500 | similar to T cell receptor beta chain |
| | | | hcvbl2 |
| Cfa.2725.1.A1_at | 1.37 | 0.07435 | — |
| Cfa.16405.1.S1_s_at | 1.37 | 0.00352 | similar to SMC5 protein |
| Cfa.18609.2.S1_at | 1.38 | 0.08848 | Transcribed locus, strongly similar to |
| | | | NP_620634.1 mitogen-activated |
| | | | protein kinase |
| Cfa.1688.1.S1_at | 1.38 | 004642 | similar to KIAA1712 protein |
| Cfa.5225.1.A1_s_at | 1.38 | 0.01976 | LOC478533 |
| Cfa.766.1.A1_at | 1.39 | 0.03803 | — |
| CfaAffx.13375.1.S1_at | 1.39 | 0.03177 | similar to Ectonucleoside |
| | | | triphosphate diphosphohydrolase 1 |
| | | | (NTPDasef)(Ecto-ATP |
| Cfa.16629.1.A1_at | 1.40 | 0.03612 | Transcribed locus |
| Cfa.15196.1.A1_at | 1.40 | 0.07081 | Transcribed locus |
| CfaAffx.8698.1.S1_at | 1.40 | 0.03564 | — |
| Cfa.9245.1.A1_at | 1.41 | 0.01794 | similar to zinc finger protein 300 |
| Cfa.11280.1.A1_at | 1.41′ | 0.08865 | similar to hypothetical protein |
| | | | B230397C21 |
| CfaAffx.5058.1.S1_s_at | 1.41 | 0.02402 | similar to hypothetical protein |
| | | | MGC48625 |
| Cfa.5819.1.A1_s_at | 1.42 | 0.05806 | — |
| Cfa.1845.1.A1_at | 1.42 | 0.02648 | — |
| CfaAffx.1750.1.S1_s_at | 1.42 | 0.00329 | similar to sperm associated antigen 1 |
| Cfa.2353.1.A1_at | 1.43 | 0.00085 | similar to hypothetical protein |
| | | | MGC48625 |
| Cfa.413.1.S1_at | 1.43 | 0.02359 | similar to core 1 UDP-galactose:N- |
| | | | acetylgalactosamine-alpha-R beta |
| | | | 1,3-galactosy |
| Cfa.18816.1.S1_at | 1.44 | 0.01551 | similar to caspase 6 isoform alpha |
| | | | preproprotein |
| Cfa.3472.1.S2_at | 1.44 | 0.04448 | frizzled homolog 6 (Drosophila) |
| Cfa.9971.1.S1_at | 1.45 | 0.03667 | similar to lactamase, beta 2 |
| CfaAffx.5057.1.S1_s_at | 1.45 | 0.00511 | similar to hypothetical protein |
| | | | MGC48625 |
| CfaAffx.19115.1.S1_at | 1.45 | 0.06192 | similar to Synaptonemal complex |
| | | | protein 2 (SCP-2 protein) |
| | | | (Synaptonemal complex |
| Cfa.8912.1.A1_at | 1.46 | 0.03398 | Transcribed locus |
| Cfa.3402.2.A1_at | 1.46 | 0.03291 | Transcribed locus, strongly similar to |
| | | | XP_346695.1 PREDICTED: |
| | | | hypothetical protein |
| CfaAffx.17619.1.S1_at | 1.46 | 0.04617 | similar to geranylgeranyl |
| | | | diphosphate synthase 1 /// |
| | | | LOC479198 |
| Cfa.1821.1.S1_at | 1.47 | 0.06437 | Transcribed locus |
| CfaAffx.7767.1.S1_s_at | 1.47 | 0.02544 | similar to DNA methyltransferase 2 |
| | | | isoform b |
| Cfa.2802.1.S1_at | 1.48 | 0.02029 | Transcribed locus |
| CfaAffx.5916.1.S1_at | 1.48 | 0.05019 | — |
| Cfa.14225.1.A1_s_at | 1.49 | 0.01832 | similar to protein phosphatase 1, |
| | | | regulatory subunit 15A |
| Cfa.5372.1.A1_at | 1.50 | 0.05451 | — |
| CfaAffx.22639.1.S1_s_at | 1.53 | 0.02181 | similar to T-cell receptor alpha chain |
| | | | /// similar to T-cell receptor alpha |
| | | | chain |
| Cfa.10872.1.A1_at | 1.57 | 0.01978 | Transcribed locus |
| Cfa.1079.1.S1_at | 1.59 | 0.00771 | similar to ankyrin repeat domain 30A |
| Cfa.1625.1.A1_at | 1.65 | 0.01148 | Transcribed locus |
| Cfa.3631.1.A1_at | 1.80 | 0.00909 | Cubilin |

All of the references cited herein and appended hereto, including patents, patent applications, literature publications, and the like, are hereby incorporated in their entireties by reference.

What is claimed is:

1. A method for diagnosing an arthritic condition in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing an arthritic condition, and the control sample is from a companion animal not having an arthritic condition, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), WW domain-containing oxidoreductase (WWOX), IL-1F, IL-6, IFN-G, CPII, BAP, NTX, HAS2, Collagen type II A1, and PGE2; and (b) identifying the companion animal corresponding to the test sample as being at risk of, or being predisposed to, developing an arthritic condition when expression of said one or more biomarker is found to be differentially expressed in the test sample compared to the control sample.

2. A method of claim 1, wherein the step of comparing the level of differential expression comprises the steps of: (i) preparing a first sample for differential expression analysis from a test companion animal suspected of having a risk of, or predisposition to, developing an arthritic condition; (ii) preparing a second sample for differential expression analysis from a companion animal having no symptoms of an arthritic condition; (iii) hybridizing the first sample to a first array of hybridization probes to detect a first set of hybridization signals; (iv) hybridizing the second sample to a second array of hybridization probes identical to the first array in step (iii) to detect a second set of hybridization signals in an otherwise similar process to step (iii); and (v) comparing the first and second sets of hybridization signals.

3. The method of claim 1, wherein the arthritic condition is selected from the group consisting of osteoarthritis, rheumatoid arthritis, ankylosing spondylitis, psoriatic arthritis or local joint inflammation.

4. The method of claim 3, wherein the arthritic condition is osteoarthritis.

5. The method of claim 1, wherein the companion animal is a canine.

6. The method of claim 1, wherein the companion animal is a feline.

7. The method of claim 1, wherein the test sample is a blood sample.

8. The method of claim 1, wherein the differential expression of step (b) is at least a 1.3-fold differential.

9. The method of claim 1, wherein the differential expression of step (b) is determined with a panel of one or more antibodies that specifically bind to said one or more biomarker.

10. The method of claim 1, wherein the differential expression of step (b) is determined by immunoassay to detect said one or more biomarker.

11. The method of claim 10, wherein the immunoassay is selected from the group consisting of a competitive binding assay, a non-competitive binding assay, a radioimmunoassay, an enzyme linked immunosorbent assay (ELISA), a sandwich assay, a precipitin reaction, a gel diffusion immunodiffusion assay, an agglutination assay, a fluorescent immunoassay, chemiluminescence immunoassay, immunoPCR immunoassay, a protein A or protein G immunoassay, Northern blot analysis, Western blot analysis, Luminex™×MAP™ detection, and an immunoelectrophoresis assay.

12. The method of claim 1, wherein said one or more biomarker is selected from the group consisting of HAS2, Collagen type II A1, PGE2 and IL-6.

13. A method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is from a companion animal not having a gastrointestinal inflammatory disorder, wherein said one or more biomarker is selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), WW domain-containing oxidoreductase (WWOX), IL-1F, IL-6, IFN-G, CPII, BAP, NTX, HAS2, Collagen type II A1, and PGE2; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

14. A method for diagnosing a gastrointestinal inflammatory disorder in a companion animal, comprising the steps of: (a) comparing the level of differential expression of one or more biomarker in a test sample to the level of expression of the same one or more biomarker in a control sample, wherein the test sample is from a companion animal suspected of being at risk of, or being predisposed to, developing a gastrointestinal inflammatory disorder, and the control sample is from a companion animal not having a gastrointestinal inflammatory disorder, wherein said one or more biomarker is selected from the group consisting of: CUBN, TLR, FOLR, FN1, TLN, NUDT9, TA1, PLCB4, ADD3, TLN1, CEACAM8, TOMIL2, ITGA4, FLNA, LIMS1, IL18R, SOD1, AP3S2, SenP6, USP1, HTR2A, HMGB1, Sept4, PLA2G7, DHPR, RAD23A, ACSL1, G6PD, GALT, LR4, LITAF, HIST1H2BE, HLA-A, HLA-B, TAC3, CACB4, CDC25A, BPI, or TUBB2A; and (b) identifying the companion animal corresponding to the test sample as being at risk of, or being predisposed to, developing a gastrointestinal inflammatory disorder when expression of said one or more biomarker is found to be differentially expressed in the test sample compared to the control sample.

15. The method of claim 13, wherein said gastrointestinal inflammatory disorder is selected from the group consisting of chronic diarrhea and an inflammatory bowel disorder.

16. The method of claim 13, wherein the gastrointestinal inflammatory disorder is chronic diarrhea.

17. The method of claim 13, wherein the gastrointestinal inflammatory disorder is an inflammatory bowel disorder.

18. The method of claim 13, wherein the companion animal is a canine.

19. The method of claim 13, wherein the companion animal is a feline.

20. The method of claim 13, wherein the test sample is a blood sample.

21. The method of claims 13, wherein the differential expression is at least a 1.3-fold differential.

22. The method of claim 13, wherein the differential expression is determined with an array of one or more hybridization probes.

23. The method of claim 13, wherein the differential expression is determined with a panel of one or more antibodies that specifically bind to said one or more biomarker.

24. The method of claim 13, wherein the differential expression is determined by immunoassay to detect said one or more biomarker.

25. The method of claim 13, wherein the immunoassay is selected from the group consisting of a competitive binding assay, a non-competitive binding assay, a radioimmunoassay, an enzyme linked immunosorbent assay (ELISA), a sandwich assay, a precipitin reaction, a gel diffusion immunodiffusion assay, an agglutination assay, a fluorescent immunoassay, chemiluminescence immunoassay, immunoPCR immunoassay, a protein A or protein G immunoassay, Northern blot analysis, Western blot analysis, Luminex™×MAP™ detection, and an immunoelectrophoresis assay.

26. A method of identifying an ingredient of a pet food composition suitable to prevent, ameliorate the symptoms of, or treat an arthritic condition or gastrointestinal inflammatory disorder in a companion animal, said method comprising the steps of: (a) contacting a first population of cells of said companion animal capable of expressing one or more biomarker selected from the group consisting of: noclin (OLFM I), Bcl-2 associated transcription factor (BCLAF1), stress-70 protein (HSPA9B), caspase 3, caspase 6, ribonucleoside-diphosphate reductase M2 subunit B (RPM2B), collaborates/cooperates with ARF (alternate reading frame) (CARF), insulin-like growth factor-binding protein 6 precursor (IGFBP6), transcobalamin (TCN 1), retinol binding protein 4 (RBP4), inducible T-cell co-stimulator precursor (ICOS), interleukin-1 receptor type II (IL1R2), interleukin-18 binding protein precursor (IL-18BP), cubilin, prolyl 4-hydroxylase alpha-1 and alpha-2 subunit precursor (P4HA), baculoviral IAP repeat-containing protein 3 (BIRC3: c-IAP2), growth arrest and DNA-damage inducible protein 34 (GADD34), regulatory (inhibitor) subunit 15A (PPPIR15A), ring finger protein 139 (TRCB=RNF139), talin (TLN I), topoisomerase (TOP2), endothelial monocyte-activating polypeptide 2 (EMAPII or SCYE), natural cytoxicitytriggering receptor 3 (NCR3), calreticulin precursor (CALR), WW domain-containing oxidoreductase (WWOX), IL-1F, IL-6, IFN-G, CPII, BAP, NTX, HAS2, Collagen type II A1, and PGE2; (b) collecting a first sample from said first population of cells, said sample containing a quantity of said one or more biomarker; (c) determining the amount of said one or more biomarker in said first sample; (d) comparing the amount of said one or more biomarker is said first sample to the amount of the same one or more biomarker present in a second sample of a corresponding second control population of cells of a companion animal that have not been contacted with said test ingredient; wherein if the amount of said one or more biomarker in said first sample is differentially expressed relative to the amount of said one or more biomarker in said second sample, the component is suitable to prevent, ameliorate the symptoms of, or treat, an arthritic condition in a companion animal.

27. The method of claim 26, wherein the ingredient is a nutrient.
